(19) United States
(12) Patent Application Publication
(10) Pub. No.: US 2023/0165139 A1
(43) Pub. Date: May 25, 2023

(54) COMPOUND FOR ORGANIC ELECTRIC ELEMENT, ORGANIC ELECTRIC ELEMENT USING SAME, AND ELECTRONIC DEVICE THEREOF

(71) Applicant: DUK SAN NEOLUX CO., LTD., Cheonan-si, Chungcheongnam-do (KR)

(72) Inventors: Hyoung Keun PARK, Cheonan-si, Chungcheongnam-do (KR); Hyun Ji OH, Cheonan-si, Chungcheongnam-do (KR); Dae Sic KIM, Cheonan-si, Chungcheongnam-do (KR); Sun Hee LEE, Cheonan-si, Chungcheongnam-do (KR); Soung Yun MUN, Cheonan-si, Chungcheongnam-do (KR); Bum Sung LEE, Cheonan-si, Chungcheongnam-do (KR); Guang Ming LI, Cheonan-si, Chungcheongnam-do (KR); Jung Geun LEE, Cheonan-si, Chungcheongnam-do (KR)

(73) Assignee: DUK SAN NEOLUX CO., LTD., Cheonan-si, Chungcheongnam-do (KR)

(21) Appl. No.: 17/995,678

(22) PCT Filed: Apr. 8, 2021
(86) PCT No.: PCT/KR2021/004434

§ 371 Date: Oct. 6, 2022

(30) Foreign Application Priority Data
Apr. 10, 2020 (KR) 10-2020-0044003
Mar. 16, 2021 (KR) 10-2021-0034201

(51) Int. Cl. H10K 85/60; H10K 50/19; C07D 307/91; C07C 211/54; C07C 209/68; C07D 333/50

(52) U.S. Cl. CPC H10K 85/636; H10K 50/19; C07D 307/91; H10K 85/633; C07C 211/54; C07C 209/68; C07D 333/50; H10K 50/15

(57) ABSTRACT

Provided are a compound which can improve the luminous efficiency, stability, and service life of an element; an organic electric element using same; and an electronic device thereof.

### BACKGROUND

### Technical Field

The present invention relates to a compound for an organic electric element, an organic electric element using same, and an electronic device thereof.

### Background Art

The flat panel display device plays a very important role in supporting the advanced image information society based on the Internet, which is showing rapid growth in recent years. In particular, an organic electroluminescent device (organic EL device) capable of low-voltage driving as a self-emission type has superior viewing angles and contrast ratios compared to liquid crystal displays (LCDs), which are the mainstream of flat panel display devices, and does not require a backlight, so it can be lightweight and thin, and has advantages in terms of power consumption. In addition, it is attracting attention as a next-generation display device because of its fast response speed and wide color reproduction range. In general, an organic EL device is formed on a glass substrate in the order of an anode made of a transparent electrode, an organic thin film including a light emitting region, and a metal electrode (cathode). In this case, the organic thin film comprises a hole injection layer (HIL), a hole transport layer (HTL), an electron transport layer (ETL) or an electron injection layer (EIL) in addition to an emitting layer (EML) and further include an electron blocking layer (EBL) or a hole blocking layer (HBL), and an emitting auxiliary layer due to the light emitting characteristics of the emitting layer. When an electric field is applied to the organic EL device having such a structure, holes are injected from the anode and electrons are injected from the cathode, and the injected holes and electrons pass through the hole transport layer and the electron transport layer, respectively, and recombine in the emitting layer to form light emitting excitons. The formed luminescent excitons emit light while transitioning to ground states, at this time, in order to increase the efficiency and stability of the light emitting state, a light emitting dye (guest) is also doped into the emitting layer (host). In order to utilize such an organic electronic device in various display media, the lifespan of the element is more important than anything else, and various studies are being conducted to increase the lifespan of the organic electric element. In particular, for excellent lifespan characteristics of organic electric devices, various studies are being conducted on organic materials inserted into a buffer layer such as a hole transport layer or an emitting auxiliary layer, and to this end, a material for a hole injection layer and a hole transport layer with high uniformity and low crystallinity when forming a thin film after deposition while providing high hole transport properties from the anode to the organic layer is required.

It is necessary to develop a material for a hole injection layer and a hole transport layer not only having stable characteristics against Joule heating generated during device driving, that is, a high glass transition temperature, but also delaying the penetration and diffusion of metal oxide from the anode electrode (ITO), which is one of the causes of shortening the lifespan of organic electric devices. In addition, it is reported that the low glass transition temperature of the hole transport layer material greatly affects the device lifespan according to the characteristic that the uniformity of the thin film surface is collapsed during device driving. In addition, in the formation of OLED devices, the deposition method is the mainstream, and a material that can withstand this deposition method for a long time, that is, a material with strong heat resistance characteristics is required.

In particular, it is urgent to overcome the problems of power consumption and lifespan, as the main overcoming challenges of organic light emitting diodes are enlarged in panel sizes of mobile phones and tablet PCs.

However, as a hole transport layer material, it is difficult to overcome the driving voltage and lifetime at the same time. The reason is that, in order to lower the driving voltage, materials with excellent hole transport ability, ie, high hole mobility, have a planar structure rich in electrons in most cases. For example, naphthyl, fluorene and phenanthrene and the like.

However, when a compound with the above structure is introduced into a hole transport material as a substituent, the hole mobility increases up to a certain number and has a good effect on the lifespan, but if the number of introductions in the molecule is increased to reach the low voltage driving target required in the current industry, the driving voltage is lowered and low voltage driving is possible, but the lifespan characteristics are rapidly deteriorated.

The reason for this is that, in the case of molecules in which electron-rich planar structures are excessively introduced, holes are trapped between the plate-like structures and stabilized when a constant current is continuously supplied during device lifespan evaluation, this lowers the hole mobility and eventually increases the driving voltage to apply a constant current, resulting in a sharp deterioration in device lifespan. It is expressed by the following formula.

$$J = \frac{9}{8} ε µ \frac{{V}^{2}}{{d}^{3}} θ = \frac{9}{8} ε µ \frac{1}{d} {F}^{2} θ$$

(J=Space Charge limited current, ε=Permissibility, μ=Mobility Coefficient, θ=Charge Trap Coefficient (Free Carrier/Total Carrier), V=Voltage, d=Thickness)

As the number of free carriers decreases due to the trap phenomenon, the θ value decreases, therefore, in the current-driven organic light emitting device that requires a constant current, the driving voltage is increased, which can have a very fatal result on the lifespan. Therefore, as described above, the introduction of an electron-rich plate-like structure capable of increasing hole mobility over a certain amount adversely affects the lifespan, so the possibility of lowering the driving voltage by using it is not great.

Accordingly, the present inventors confirmed that the compound substituted with deuterium showed many thermodynamic behaviors compared to the unsubstituted compound, and that, among these thermodynamic properties, when the iridium compound is substituted with deuterium, the properties vary according to the difference in carbon, hydrogen, and carbon and deuterium bond lengths, and that the compound composed of deuterium can have higher luminous efficiency due to the weakening of the intermolecular van der Waals force generated by the shorter bond length compared to the compound not substituted with deuterium.

However, a method of lowering the driving voltage by substituting with deuterium, that is, increasing the hole transport Mobility of the hole transport material, has not been studied much at present, and the prior art that demonstrates the effect according to a specific deuterium substitution rate has not yet been reported. Moreover, the conventionally reported general deuterium substitution method has a disadvantage in that it is difficult to control the substitution rate.

### DETAILED DESCRIPTION OF THE INVENTION

### Summary

For the purpose of solving the problems of the above-mentioned background art, in the present invention, deuterium is substituted in a specific ratio of 74% to 98% to an amine-based compound having long lifespan, thereby completing a device with a long lifespan, in order to realize a long-life device, which is a required characteristic of an organic electric device.

Accordingly, an object of the present invention is to provide a compound deuterated at a specific ratio, an organic electric element using same, and an electronic device thereof.

### Technical Solution

The present invention provides a 74% to 98% deuterated compound represented by Formula A.

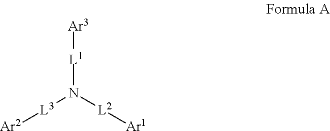

In another aspect, the present invention provides a method for preparing the deuterated compound represented by Formula A.

In another aspect, the present invention provides an organic electric element and an electronic device comprising the compound represented by Formula A.

### Effects of the Invention

By using the compound according to the present invention, high luminous efficiency, low driving voltage and high heat resistance of the element can be achieved, and color purity and lifespan of the element can be greatly improved.

### BRIEF DESCRIPTION OF THE DRAWINGS

FIG. 1 to FIG. 3 are exemplary views of an organic electroluminescent device according to the present invention.

FIG. 4 shows a Formula according to an aspect of the present invention.

| | | | |
|---|---|---|---|
| 100, 200, 300: | organic electric element | 110: | the first electrode |
| 120: | hole injection layer | 130: | hole transport layer |
| 140: | emitting layer | 150: | electron transport layer |
| 160: | electron injection layer | 170: | second electrode |
| 160: | electron transport layer | 170: | electron injection layer |
| 180: | light efficiency enhancing Layer | 210: | buffer layer |
| 220: | emitting auxiliary layer | 320: | first hole injection layer |
| 330: | first hole transport layer | 340: | first emitting layer |
| 350: | first electron transport layer | 360: | first charge generation layer |
| 361: | second charge generation layer | 420: | second hole injection layer |
| 430: | second hole transport layer | 440: | second emitting layer |
| 450: | second electron transport layer | CGL: | charge generation layer |
| ST1: | first stack | ST2: | second stack |

### DETAILED DESCRIPTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected coupled” or “connected” between each component.

As used in the specification and the accompanying claims, unless otherwise stated, the following is the meaning of the term as follows.

Unless otherwise stated, the term “halo” or “halogen”, as used herein, includes fluorine, bromine, chlorine, or iodine.

Unless otherwise stated, the term “alkyl” or “alkyl group”, as used herein, has a single bond of 1 to 60 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “alkenyl” or “alkynyl”, as used herein, has double or triple bonds of 2 to 60 carbon atoms, but is not limited thereto, and includes a linear or a branched chain group.

Unless otherwise stated, the term “cycloalkyl”, as used herein, means alkyl forming a ring having 3 to 60 carbon atoms, but is not limited thereto.

Unless otherwise stated, the term “alkoxyl group”, “alkoxy group” or “alkyloxy group”, as used herein, means an oxygen radical attached to an alkyl group, but is not limited thereto, and has 1 to 60 carbon atoms.

Unless otherwise stated, the term “aryloxyl group” or “aryloxy group”, as used herein, means an oxygen radical attached to an aryl group, but is not limited thereto, and has 6 to 60 carbon atoms.

The terms “aryl group” and “arylene group” used in the present invention have 6 to 60 carbon atoms, respectively, unless otherwise specified, but are not limited thereto. In the present invention, an aryl group or an arylene group means a single ring or multiple ring aromatic, and includes an aromatic ring formed by an adjacent substituent joining or participating in a reaction.

For example, the aryl group may be a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heterocyclic group”, as used herein, contains one or more heteroatoms, but is not limited thereto, has 2 to 60 carbon atoms, includes any one of a single ring or multiple ring, and may include heteroaliphadic ring and heteroaromatic ring. Also, the heterocyclic group may also be formed in conjunction with an adjacent group.

Unless otherwise stated, the term “heteroatom”, as used herein, represents at least one of N, O, S, P, or Si.

Also, the term “heterocyclic group” may include a ring including SO₂ instead of carbon consisting of cycle. For example, “heterocyclic group” includes the following compound.

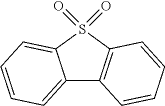 [SMILES: O=S1(=O)c2ccccc2-c2ccccc21]

Unless otherwise stated, the term “fluorenyl group” or “fluorenylene group”, as used herein, means a monovalent or divalent functional group, in which R, R′ and R″ are all hydrogen in the following structures, and the term “substituted fluorenyl group” or “substituted fluorenylene group” means that at least one of the substituents R, R′, R″ is a substituent other than hydrogen, and include those in which R and R′ are bonded to each other to form a spiro compound together with the carbon to which they are bonded.

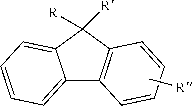

The term “spiro compound”, as used herein, has a ‘spiro union’, and a spiro union means a connection in which two rings share only one atom. At this time, atoms shared in the two rings are called ‘spiro atoms’, and these compounds are called ‘monospiro-’, ‘di-spiro-’ and ‘tri-spiro-’, respectively, depending on the number of spiro atoms in a compound.

Unless otherwise stated, the term “aliphatic”, as used herein, means an aliphatic hydrocarbon having 1 to 60 carbon atoms, and the term “aliphatic ring”, as used herein, means an aliphatic hydrocarbon ring having 3 to 60 carbon atoms.

Unless otherwise stated, the term “ring”, as used herein, means an aliphatic ring having 3 to 60 carbon atoms, or an aromatic ring having 6 to 60 carbon atoms, or a hetero ring having 2 to 60 carbon atoms, or a fused ring formed by the combination of them, and includes a saturated or unsaturated ring.

Other hetero compounds or hetero radicals other than the above-mentioned hetero compounds include, but are not limited thereto, one or more heteroatoms.

Also, unless expressly stated, as used herein, “substituted” in the term “substituted or unsubstituted” means substituted with one or more substituents selected from the group consisting of deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiopen group, a C₆-C₂₀ arylthiopen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group, but is not limited to these substituents.

Also, unless there is an explicit explanation, the formula used in the present invention is the same as the definition of the substituent by the exponent definition of the following formula.

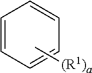

Here, when a is an integer of 0, the substituent R¹ is absent, when a is an integer of 1, the sole substituent R¹ is linked to any one of the carbon constituting the benzene ring, when a is an integer of 2 or 3, each is bonded as follows, where R¹ may be the same or different from each other, when a is an integer of 4 to 6, it is bonded to the carbon of the benzene ring in a similar manner, while the indication of the hydrogen bonded to the carbon forming the benzene ring is omitted.

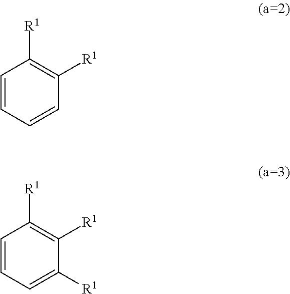

As used herein, the term “deuterated” refers to a compound or group in which deuterium is present at 100 times or more of its natural abundance level.

As used herein, the term “perdeuterated” refers to a compound or group in which all hydrogens have been replaced with deuterium. The term deuterated is synonymous with “100% deuterated”.

As used herein, the term “deutero-acid” refers to a compound capable of ionizing to donate deuterium ions to bronsted base. As used herein, deuterium-acids do not contain ionizable hydrogens.

Hereinafter, a compound according to an aspect of the present invention and an organic electric element including the same will be described.

The present invention is a method that can lower the driving voltage without introducing a plate-shaped molecular structure that adversely affects the lifespan by using a material with a good lifespan, and by using a method of substituting deuterium at an appropriate ratio, provides a method of lowering the driving voltage.

When substituted with deuterium, the zero point energy, that is, the energy of the ground state is lowered, and as the bond length of deuterium-carbon becomes shorter than that of hydrogen-carbon, the molecular hardcore volume decreases, accordingly, electrical polarizability can be reduced, and by weakening the intermolecular interaction, the thin film volume can be increased. This property can lower the crystallinity of the thin film, that is, create an amorphous state, and is generally very effective in implementing an amorphous state, which is essential to increase OLED lifespan and driving characteristics.

In addition, when a film is formed with a compound substituted with deuterium, the film is formed in an amorphous glass state that can greatly affect the hole mobility of the thin film, and this amorphous glass state can reduce the grain boundary through isotropic and homogeneous properties, thereby speeding up the flow of charges, that is, hole mobility.

The present invention provides a compound represented by Formula A deuterated to 74% to 98%.

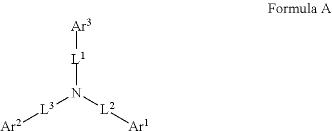

In Formula A, Formula a and Formula b, each symbol may be defined as follows.

1) L¹, L² and L³ are each independently selected from the group consisting of a single bond; a C₆-C₆₀ arylene group; fluorenylene group; a fused ring group of a C₃-C₆₀ aliphatic ring and a C₆-C₆₀ aromatic ring; and a C₂-C₆₀ heterocyclic group;

Wherein in case L¹, L² and L³ are an arylene group, they may be preferably a C₆-C₃₀ arylene group, more preferably a C₆-C₂₄ arylene group, for example, phenylene, biphenyl, naphthalene, terphenyl, etc.

Wherein in case L¹, L² and L³ are a heterocyclic group, they may be preferably a C₂˜C₃₀ heterocyclic group, more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophen, benzothienopyrimidine, benzofuropyrimidine, phenothiazine, phenylphenothiazine, etc.

Wherein in case L¹, L² and L³ are is a fused ring groups, they may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

2) Ar¹ and Ar² are each independently selected from the group consisting of a C₆-C₆₀ aryl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₆₀ alkyl group; a C₂-C₂₀ alkenyl group; a C₂-C₂₀ alkynyl group; a C₁-C₃₀ alkoxyl group; a C₆-C₃₀ aryloxy group;

-L″-N(Ar^{a})(Ar^{b}); a substituent represented by Formula a; and a substituent represented by Formula b;

Wherein in case Ar¹ and Ar² are an aryl group, they may be preferably a C₆-C₃₀ aryl group, more preferably a C₆-C₂₅ aryl group, for example, phenylene, biphenyl, naphthalene, terphenyl, etc.

Wherein in case Ar¹ and Ar² are a heterocyclic group, they may be preferably a C₂˜C₃₀ heterocyclic group, more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.

Wherein in case Ar¹ and Ar² are a fused ring groups, they may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

Wherein in case Ar¹ and Ar² are an alkyl group, they may be preferably a C₁-C₃₀ alkyl group, more preferably a C₁-C₂₄ alkyl group.

Wherein in case Ar¹ and Ar² are an alkoxyl group, they may be preferably an C₁˜C₂₄ alkoxyl group.

Wherein in case Ar¹ and Ar² are an aryloxy group, they may be preferably an C₆˜C₂₄ aryloxy group.

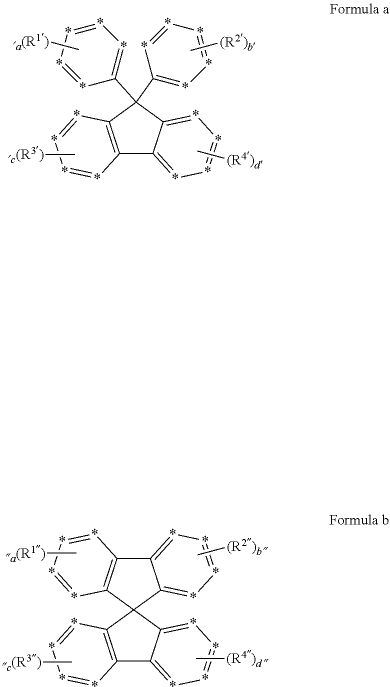

3) Ar³ is selected from the group consisting of a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₆₀ alkyl group; a C₂-C₂₀ alkenyl group; a C₂-C₂₀ alkynyl group; a C₁-C₃₀ alkoxyl group; a C₆-C₃₀ aryloxy group; -L″-N(Ar^{a})(Ar^{b});

Wherein in case Ar³ is an aryl group, it may be preferably a C₆-C₃₀ aryl group, more preferably a C₆-C₂₅ aryl group, for example, phenylene, biphenyl, naphthalene, terphenyl, etc.

Wherein in case Ar³ is a heterocyclic group, it may be preferably a C₂˜C₃₀ heterocyclic group, more preferably a C₂-C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.

Wherein in case Ar³ is a fused ring groups, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

Wherein in case Ar³ is an alkyl group, it may be preferably a C₁-C₃₀ alkyl group, more preferably a C₁-C₂₄ alkyl group.

Wherein in case Ar³ is an alkoxyl group, it may be preferably an C₁˜C₂₄ alkoxyl group.

Wherein in case Ar³ is an aryloxy group, it may be preferably an C₆˜C₂₄ aryloxy group.

4) R^{1′}, R^{2′}, R^{3′}, R^{4′}, R^{1″}, R^{2″}, R^{3″} and R^{4″} are each independently selected from the group consisting of hydrogen; deuterium; halogen; cyano group; nitro group; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₆₀ alkyl group; a C₂-C₂₀ alkenyl group; a C₂-C₂₀ alkynyl group; a C₁-C₃₀ alkoxyl group; a C₆-C₃₀ aryloxy group; and -L″-N(Ar^{a})(Ar^{b}); or in case a′, b′, c′, d′, a″, b″, c″ and d″ are 2 or more, a′, b′, c′, d′, a″, b″, c″ and d″ are each in plural being the same or different, an adjacent plurality of R^{1′}s, or a plurality of R^{2′}s, or a plurality of R^{3′}s, or a plurality of R^{4′}s, or a plurality of R^{1″}s, or a plurality of R^{2″}s, or a plurality of R^{3″}s or s, a plurality of and R^{4″}s may be bonded to each other to form a ring.

Wherein in case R^{1′}, R^{2′}, R^{3′}, R^{4′}, R^{1″}, R^{2″}, R^{3″} and R^{4″} are an aryl group, they may be preferably a C₆-C₃₀ aryl group, more preferably a C₆-C₂₅ aryl group, for example, phenylene, biphenyl, naphthalene, terphenyl, etc.

Wherein in case R^{1′}, R^{2′}, R^{3′}, R^{4′}, R^{1″}, R^{2″}, R^{3″} and R^{4″} are a heterocyclic group, they may be preferably a C₂˜C₃₀ heterocyclic group, more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.

Wherein in case R^{1′}, R^{2′}, R^{3′}, R^{4′}, R^{1″}, R^{2″}, R^{3″} and R^{4″} are a fused ring groups, they may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

Wherein in case R^{1′}, R^{2′}, R^{3′}, R^{4′}, R^{1″}, R^{2″}, R^{3″} and R^{4″} are an alkyl group, they may be preferably a C₁-C₃₀ alkyl group, more preferably a C₁-C₂₄ alkyl group.

Wherein in case R^{1′}, R^{2′}, R^{3′}, R^{4′}, R^{1″}, R^{2″}, R^{3″} and R^{4″} are an alkoxyl group, they may be preferably an C₁-C₂₄ alkoxyl group.

Wherein in case R^{1′}, R^{2′}, R^{3′}, R^{4′}, R^{1″}, R^{2″}, R^{3″} and R^{4″} are an aryloxy group, they may be preferably an C₆-C₂₄ aryloxy group.

5) a′ and b′ are each independently an integer from 0 to 5, c′, d′, a″, b″, c″ and d″ are each independently an integer from 0 to 4,

6) Any one of * is bonded to L² or L³ of Formula A,

7) wherein L″ is each independently selected from the group consisting of a single bond; a C₆-C₆₀ arylene group; fluorenylene group; a fused ring group of a C₃-C₆₀ aliphatic ring and a C₆-C₆₀ aromatic ring; and a C₂-C₆₀ heterocyclic group including at least one heteroatom of O, N, S, Si or P; and wherein Ar^{a} and Ar^{b} are each independently selected from the group consisting of a C₆-C₆₀ aryl group; fluorenyl group; a fused ring group of a C₃-C₆₀ aliphatic ring and a C₆-C₆₀ aromatic ring; and a C₂-C₆₀ heterocyclic group including at least one heteroatom of O, N, S, Si or P; Wherein in case L″ is an arylene group, it may be preferably a C₆-C₃₀ arylene group, more preferably a C₆-C₂₄ arylene group, for example, phenylene, biphenyl, naphthalene, terphenyl, etc.

Wherein in case L″ is a heterocyclic group, it may be preferably a C₂˜C₃₀ heterocyclic group, and more preferably a C₂-C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.

In case L″ is fused ring groups, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

Wherein in case Ar^{a} and Ar^{b} are an aryl group, they may be preferably a C₆-C₃₀ aryl group, more preferably a C₆-C₂₅ aryl group, for example, phenylene, biphenyl, naphthalene, terphenyl, etc.

Wherein in case Ar^{a} and Ar^{b} are a heterocyclic group, they may be preferably a C₂˜C₃₀ heterocyclic group, more preferably a C₂-C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.

Wherein in case Ar^{a} and Ar^{b} are a fused ring groups, they may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

8) wherein the aryl group, arylene group, heterocyclic group, fluorenyl group, fluorenylene group, fused ring group, alkyl group, alkenyl group, alkynyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂˜C₂₀ heterocyclic group; C₃-C₂₀ cycloalkyl group; C₇-C₂₀ arylalkyl group; and C₈-C₂₀ arylalkenyl group; and -L″-N(Ar^{a})(Ar^{b}); also the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Also, the present invention includes compounds in which Ar³ is represented by Formula Ar-1 or Ar-2.

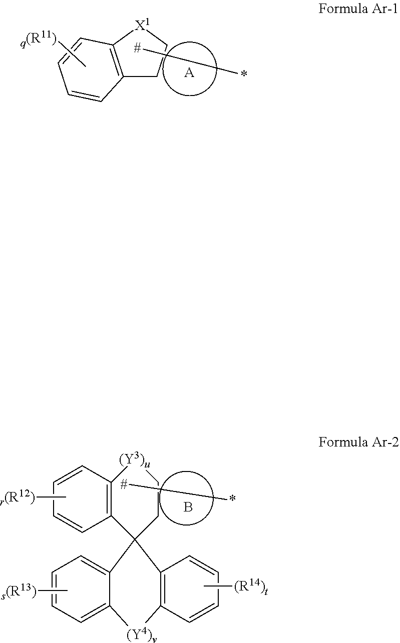

Wherein, each symbol may be defined as follows.

1) * means a position bonded to L¹ of Formula A,

2) X¹ is O, S, NR^{a} or CR^{b}R^{c},

with the proviso that when # is bonded to X¹, X¹ is N,

3) Y³ and Y⁴ are each independently single bond, O, S, NR^{d} or CR^{e}R^{f}, with the proviso that when # is bonded to Y³, Y³ is N,

4) R^{a}, R^{b}, R^{c}, R^{d}, R^{e} and R^{f} are each independently selected from the group consisting of a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; a C₆-C₆₀ aryl group; fluorenyl group; a C₂-C₆₀ heterocyclic group including at least one heteroatom of O, N, S, Si or P; or R^{a}, R^{b}, R^{c}, R^{d}, R^{e} and R^{f} are bonded to adjacent groups to form a ring.

Wherein in case R^{a}, R^{b}, R^{c}, R^{d}, R^{e} and R^{f} are an alkyl group, they may be preferably a C₁-C₃₀ alkyl group, more preferably a C₁-C₂₄ alkyl group.

Wherein in case R^{a}, R^{b}, R^{c}, R^{d}, R^{e} and R^{f} are an alkoxyl group, they may be preferably an C₁˜C₂₄ alkoxyl group.

Wherein in case R^{a}, R^{b}, R^{c}, R^{d}, R^{e} and R^{f} are an aryloxy group, they may be preferably an C₆-C₂₄ aryloxy group.

Wherein in case R^{a}, R^{b}, R^{c}, R^{d}, R^{e} and R^{f} are an aryl group, they may be preferably a C₆˜C₃₀ aryl group, more preferably a C₆˜C₂₅ aryl group, for example, phenylene, biphenyl, naphthalene, terphenyl, etc.

Wherein in case R^{a}, R^{b}, R^{c}, R^{d}, R^{e} and R^{f} are a heterocyclic group, they may be preferably a C₂˜C₃₀ heterocyclic group, more preferably a C₂-C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.

5) Ring A and ring B are each independently selected from the group consisting of a C₆-C₆₀ aryl group; a C₂-C₆₀ heterocyclic group including at least one heteroatom of O, N, S, Si or P; and a fused ring group of a C₃-C₆₀ aliphatic ring and a C₆-C₆₀ aromatic ring; or ring A and ring B may be substituted with R²¹.

Wherein in case ring A and ring B are an aryl group, they may be preferably a C₆-C₃₀ aryl group, more preferably a C₆-C₂₅ aryl group, for example, phenylene, biphenyl, naphthalene, terphenyl, etc.

Wherein in case ring A and ring B are a heterocyclic group, they may be preferably a C₂˜C₃₀ heterocyclic group, more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.

Wherein in case ring A and ring B are a fused ring groups, they may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

6) R¹¹, R¹², R¹³, R¹⁴ and R²¹ are each independently selected from the group consisting of hydrogen; deuterium; halogen; cyano group; a C₆-C₆₀ aryl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxy group; a C₆-C₃₀ aryloxy group; and -L″-N(Ar^{a})(Ar^{b}); or R¹¹, R¹², R¹³, R¹⁴ and R²¹ are bonded to adjacent groups to form a ring.

Wherein in case R¹¹, R¹², R¹³, R¹⁴ and R²¹ are an aryl group, they may be preferably a C₆-C₃₀ aryl group, more preferably a C₆-C₂₅ aryl group, for example, phenylene, biphenyl, naphthalene, terphenyl, etc.

Wherein in case ring R¹¹, R¹², R¹³, R¹⁴ and R²¹ are a heterocyclic group, they may be preferably a C₂˜C₃₀ heterocyclic group, more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.

Wherein in case R¹¹, R¹², R¹³, R¹⁴ and R²¹ are a fused ring groups, they may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

Wherein in case R¹¹, R¹², R¹³, R¹⁴ and R²¹ are an alkyl group, they may be preferably a C₁-C₃₀ alkyl group, more preferably a C₁-C₂₄ alkyl group.

Wherein in case R¹¹, R¹², R¹³, R¹⁴ and R²¹ are an alkoxy group, they may be preferably an C₁˜C₂₄ alkoxy group.

Wherein in case R¹¹, R¹², R¹³, R¹⁴ and R²¹ are an aryloxy group, they may be preferably an C₆˜C₂₄ aryloxy group.

7) wherein L″, Ar^{a} and Ar^{b} are the same as defined above,

8) q, r, s, and t are each independently an integer of 0 to 4, u and v are each independently 0 or 1, with the proviso that except when both u and v are 0, when u or v is 0, Y³ or Y⁴ is absent.

Also, Formula A is represented by Formula 1.

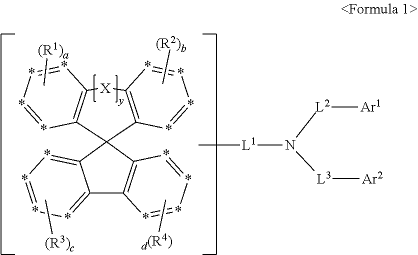

Wherein,

1) X is a single bond,

2) y is an integer of 0 or 1,

3) i) when y is 0, a and b are each independently an integer from 0 to 5,
ii) when y is 1, a and b are each independently an integer of 0 to 4,

4) c and d are each independently an integer from 0 to 4,

5) R¹, R², R³ and R⁴ are the same as the definition of R^{1′},

6) L¹, L², L³, Ar¹ and Ar² are the same as defined above, however, L¹ is bonded to any one of * in Formula 1.

Also, Formula 1 is represented by any one of Formulas 2 to 4.

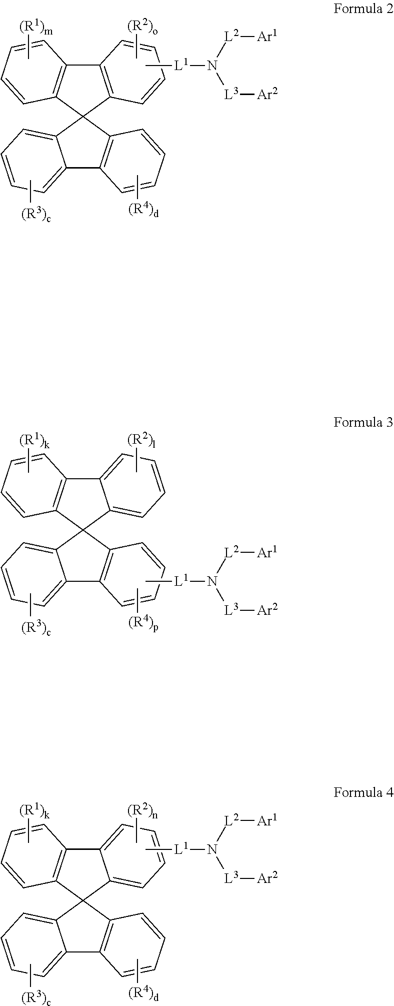

Wherein

1) R¹, R², R³, R⁴, c, d, L¹, L², L³, Ar¹ and Ar² are the same as defined above,

2) k and l are each independently an integer of 0 to 5, m and n are each independently 0 to 4, o and p are each independently 0 to 3.

Also, Formula 1 is represented by Formula 5 or Formula 6.

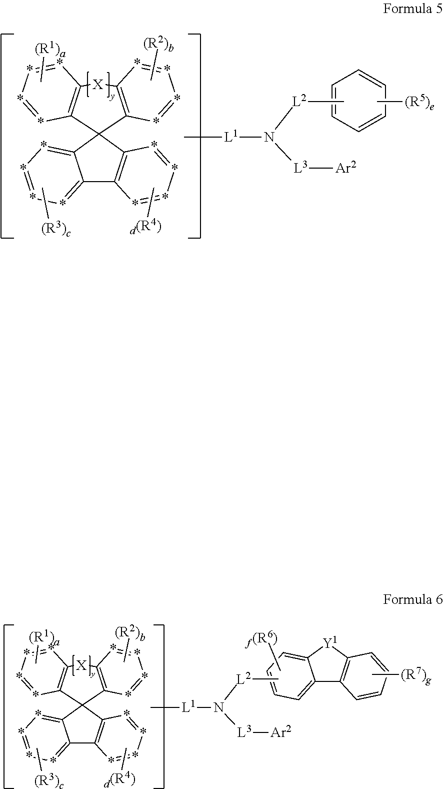

Wherein each symbols may be defined as follows.

1) R¹, R², R³, R⁴, a, b, c, d, L¹, L², L³, Ar², X and y are the same as defined above,

2) R⁵, R⁶ and R⁷ are the same as the definition of R¹,

3) e is an integer of 0 to 5, f is an integer of 0 to 3, g is an integer of 0 to 4,

4) Y¹ is O, S, CR′R″ or NR′,

5) wherein R and R are each independently selected from the group consisting of a C₆-C₆₀ aryl group; a fluorenyl group; a C₂-C₆₀ heterocyclic group including at least one heteroatom of O, N, S, Si or P; and a fused ring group of a C₃-C₆₀ aliphatic ring and a C₆-C₆₀ aromatic ring; and -L″-N(Ar^{a})(Ar^{b}); or R′ and R″ are bonded to each other to form a C₆-C₆₀ aromatic ring; a fluorenyl group; a C₂-C₆₀ heterocyclic group including at least one heteroatom of O, N, S, Si or P; a C₃-C₆₀ aliphatic ring; or a fused ring group of a C₃-C₆₀ aliphatic ring and a C₆-C₆₀ aromatic ring;

Wherein in case R′ and R″ are an aryl group, they may be preferably a C₆-C₃₀ aryl group, more preferably a C₆-C₂₅ aryl group, for example, phenylene, biphenyl, naphthalene, terphenyl, etc.

Wherein in case ring R′ and R″ are a heterocyclic group, they may be preferably a C₂˜C₃₀ heterocyclic group, more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.

Wherein in case R′ and R″ are a fused ring groups, they may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

6) wherein L″, Ar^{a} and Ar^{b} are the same as defined above.

Formula 1 is represented by any one of Formulas 7 to 15

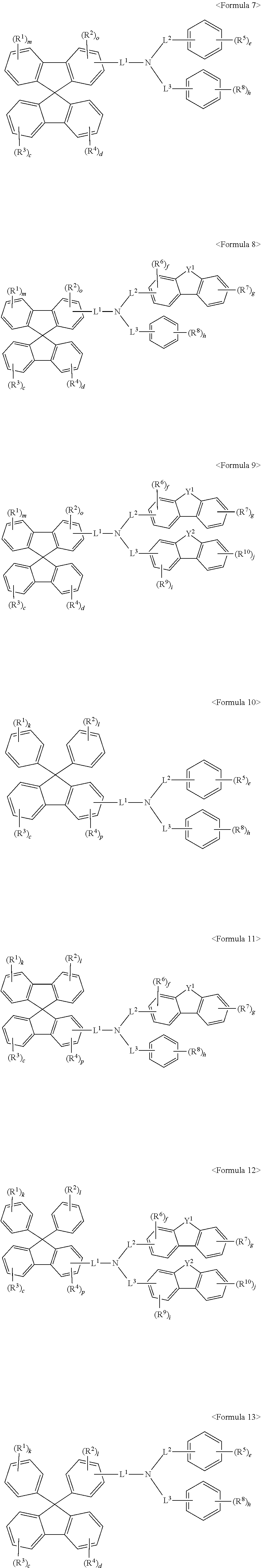

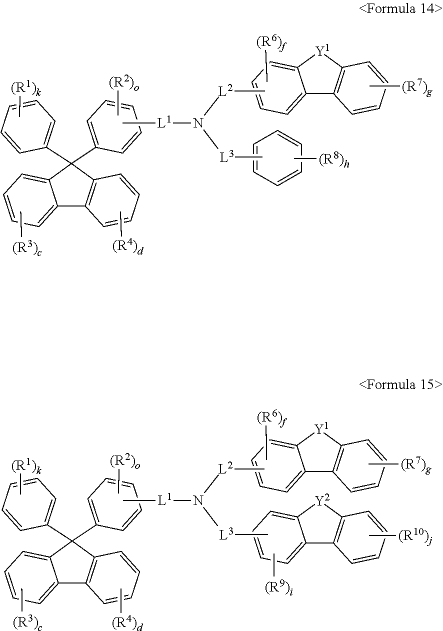

Wherein, each symbol may be defined as follows.

1) L¹, L², L³, c, d, e, f, g, k, l, m, n, o, p and Y¹ are the same as defined above,

2) R¹, R², R³, R⁴, R⁵, R⁶, R⁷, R⁶, R⁹ and R¹⁰ are the same as the definition of R¹,

3) h is an integer of 0 to 5, i is an integer of 0 to 3, j is an integer of 0 to 4,

4) Y² is O, S or CR^{x}R^{y},

5) wherein R^{x} and R^{y} are each independently selected from the group consisting of a C₁-C₅₀ alkyl group; a C₆-C₆₀ aryl group; fluorenyl group; a C₂-C₆₀ heterocyclic group including at least one heteroatom of O, N, S, Si or P; or R^{x} and R^{y} are bonded to each other to form a C₆-C₆₀ aromatic ring; a fluorenyl group; a C₂-C₆₀ heterocyclic group including at least one heteroatom of O, N, S, Si or P; a C₃-C₆₀ aliphatic ring; or a fused ring group of a C₃-C₆₀ aliphatic ring and a C₆-C₆₀ aromatic ring;

Wherein in case R^{x} and R^{y} are an alkyl group, they may be preferably a C₁-C₃₀ alkyl group, more preferably a C₁-C₂₄ alkyl group.

Wherein in case R^{x} and R^{y} are an aryl group, they may be preferably a C₆-C₃₀ aryl group, more preferably a C₆-C₂₅ aryl group, for example, phenylene, biphenyl, naphthalene, terphenyl, etc.

Wherein in case R^{x} and R^{y} are a heterocyclic group, they may be preferably a C₂˜C₃₀ heterocyclic group, more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.

Also, Formula A is represented by any one of the following compounds P-1 to P-20 and P-83 to P-130.

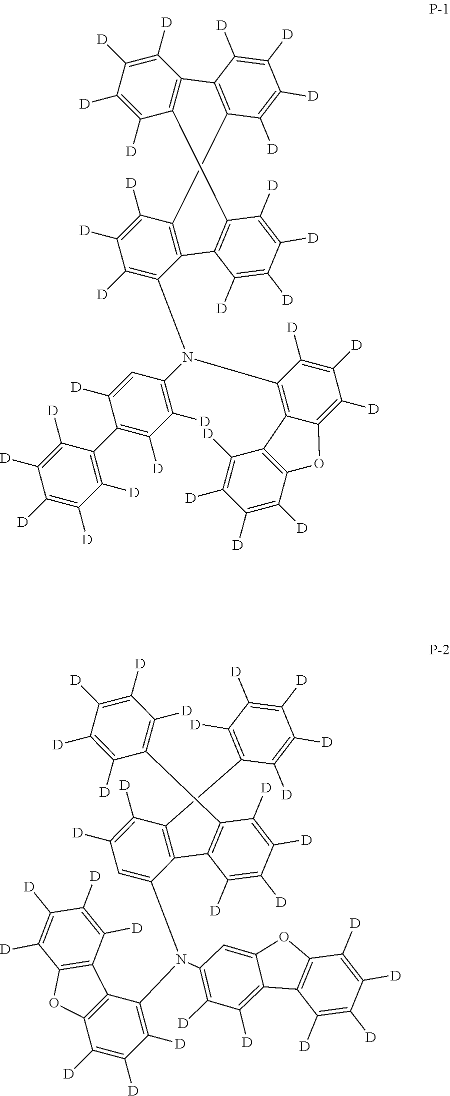

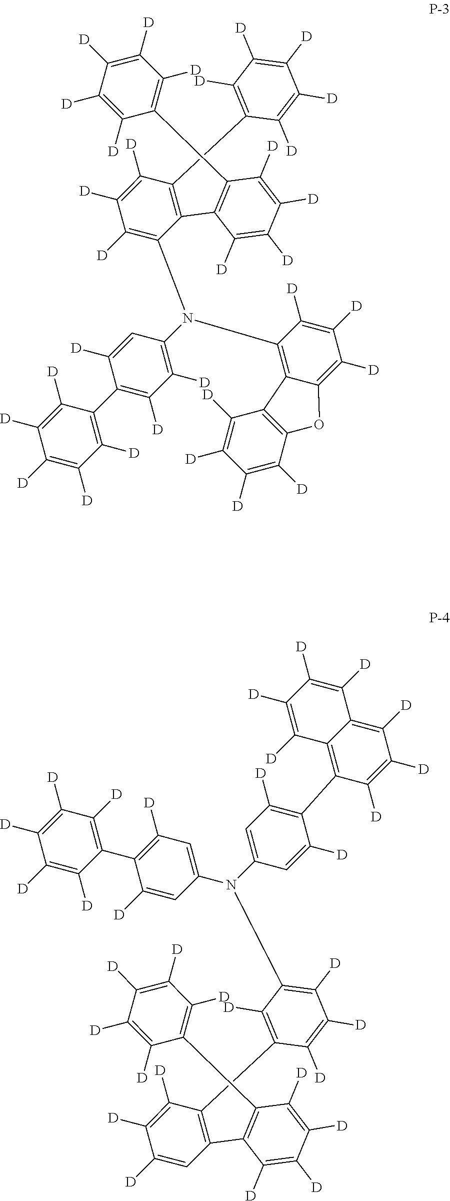

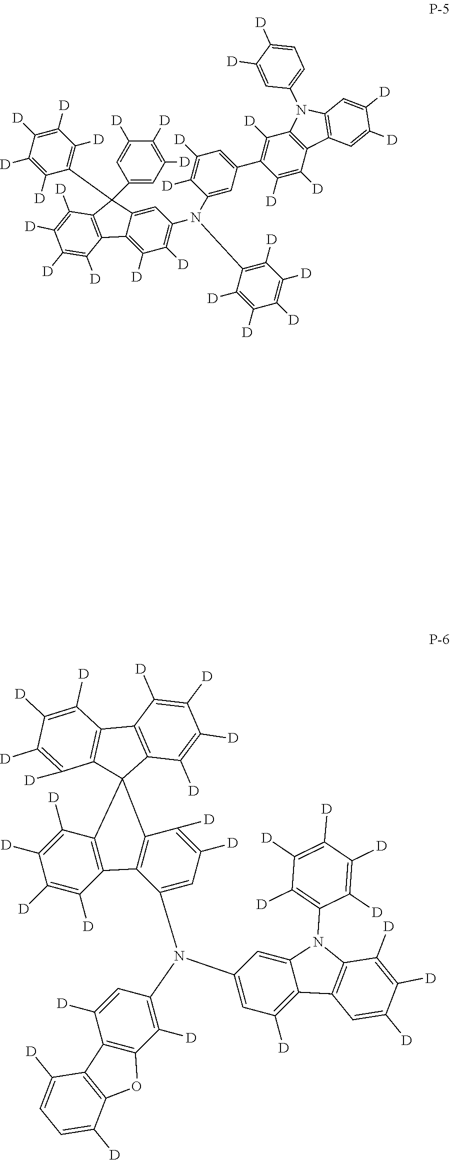

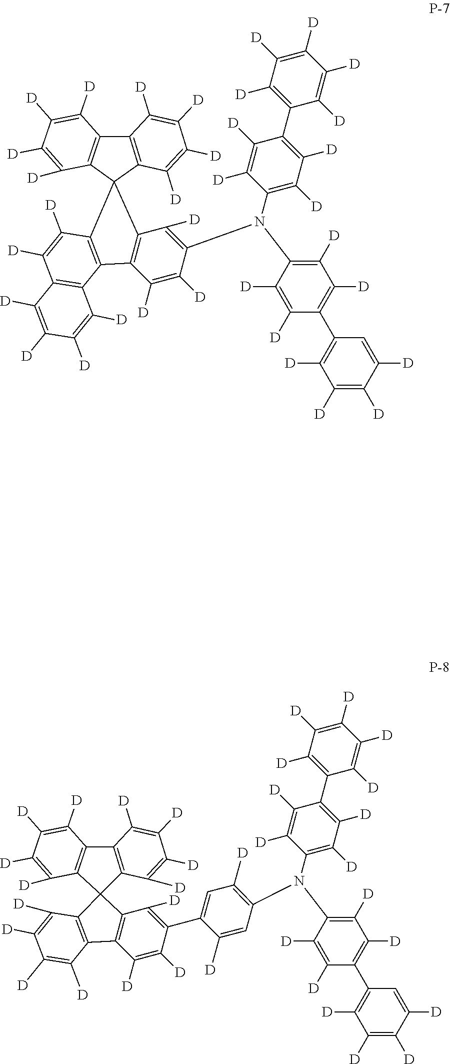

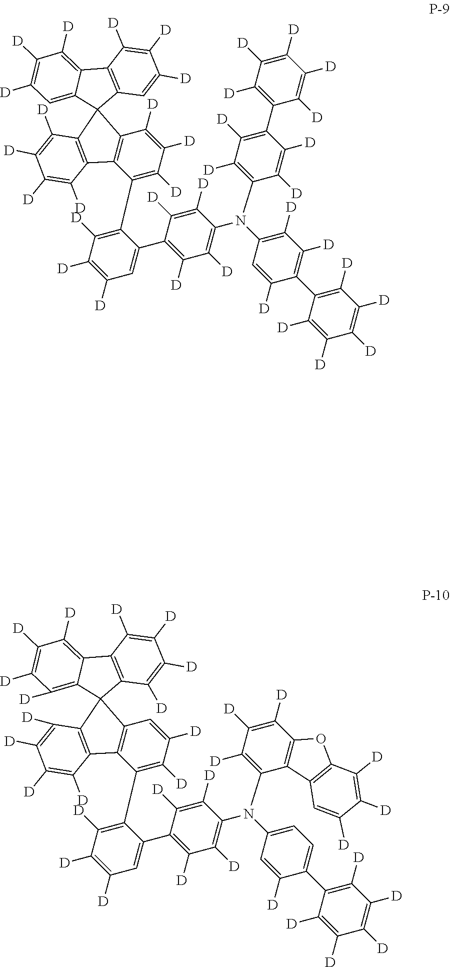

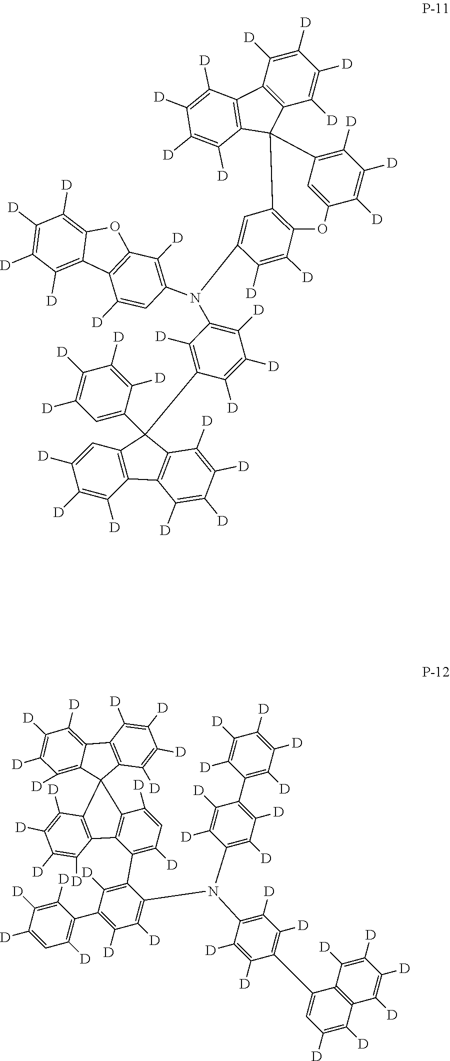

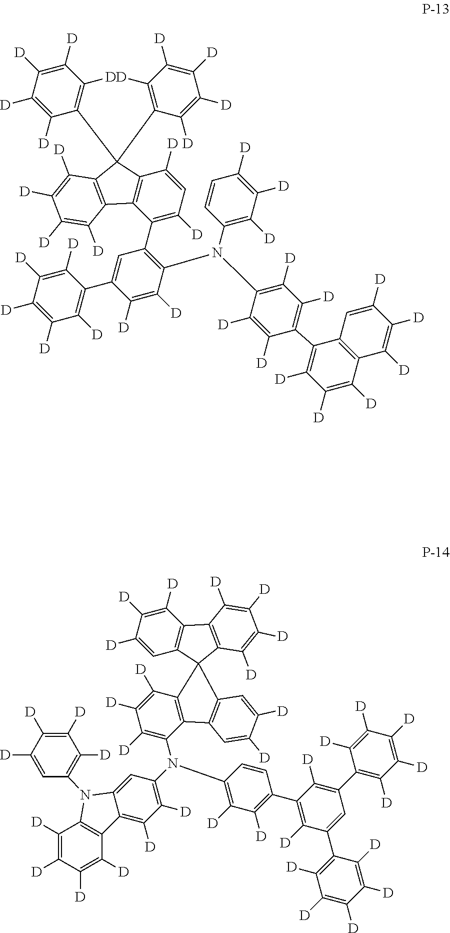

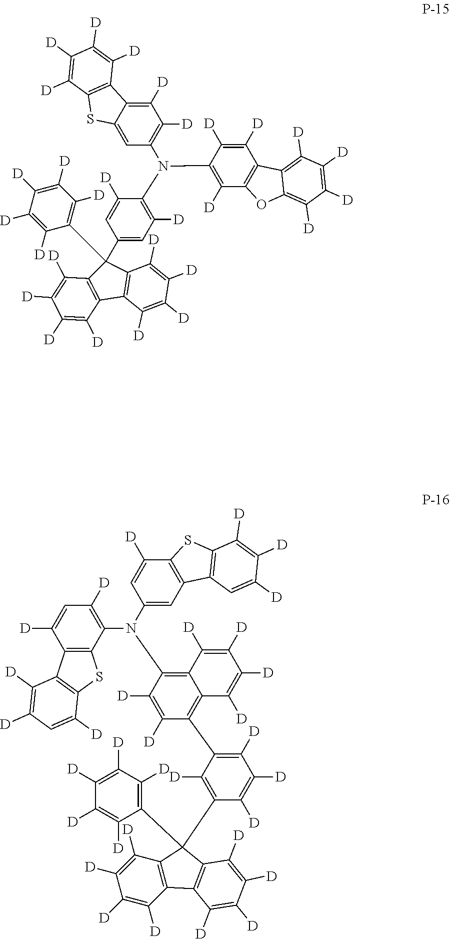

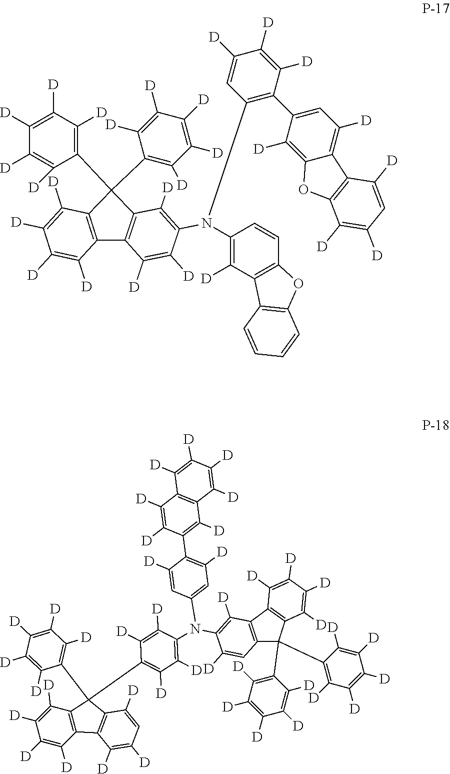

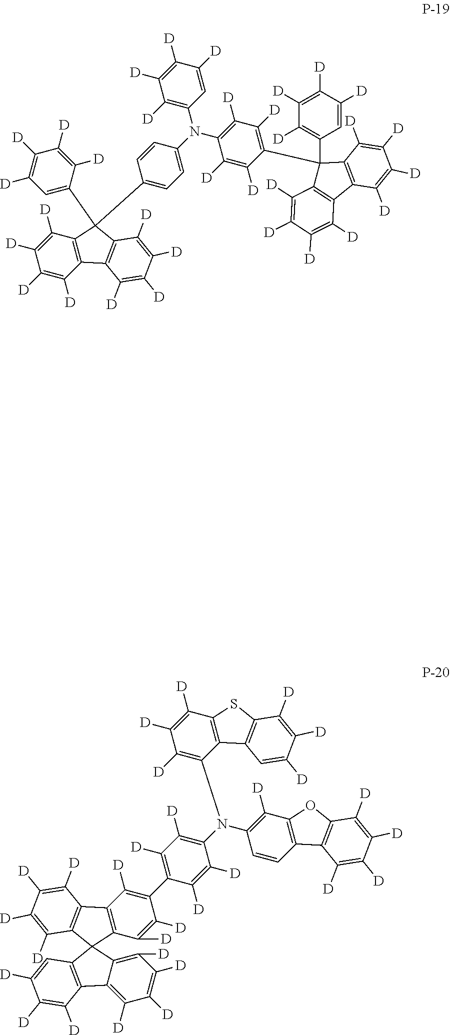

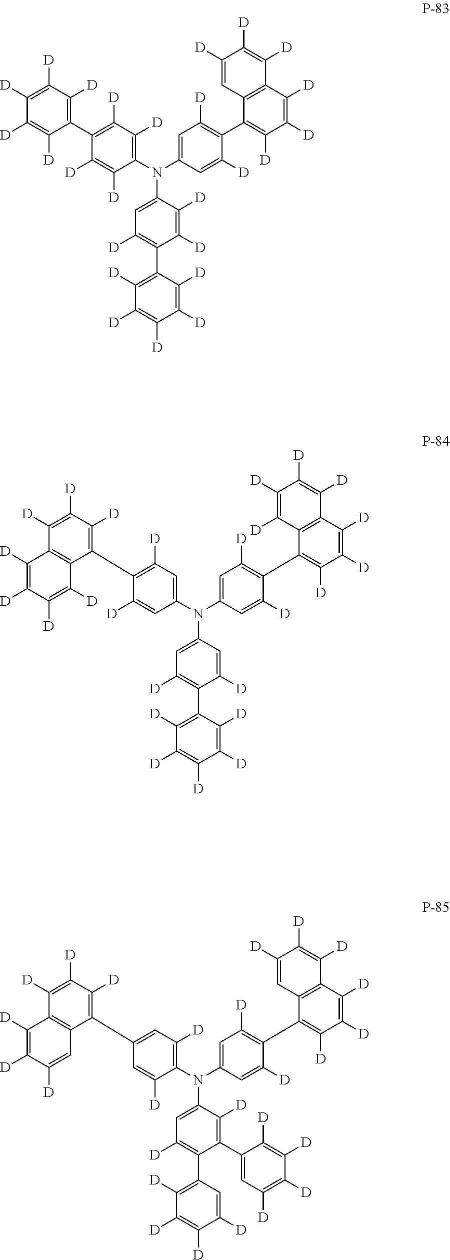

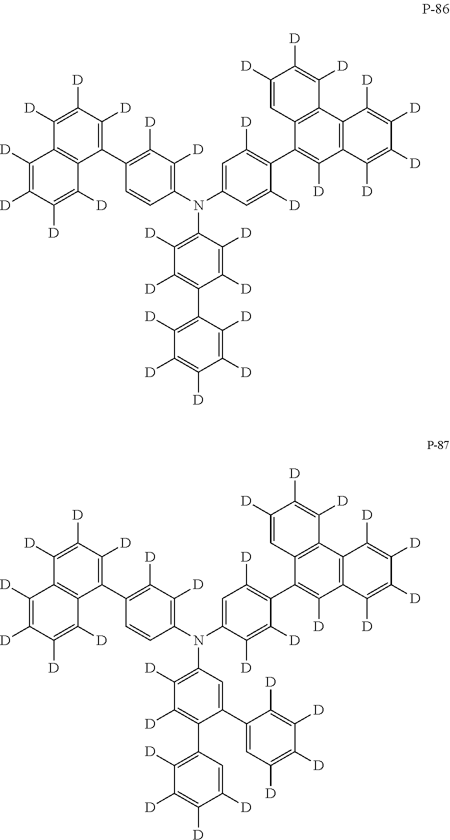 [SMILES: [2H]c1cc2c(-c3c([2H])cc(N(c4ccc(-c5c([2H])c([2H])c([2H])c6c([2H])c([2H])c([2H])c([2H])c56)c([2H])c4[2H])c4c([2H])c([2H])c(-c5c([2H])c([2H])c([2H])c([2H])c5[2H])c([2H])c4[2H])cc3[2H])c([2H])c3c([2H])c([2H])c([2H])c([2H])c3c2c([2H])c1[2H] ; [2H]c1cc(-c2cc(N(c3ccc(-c4c([2H])c([2H])c([2H])c5c([2H])c([2H])c([2H])c([2H])c45)c([2H])c3[2H])c3cc([2H])c(-c4c([2H])c5c([2H])c([2H])c([2H])c([2H])c5c5c([2H])c([2H])c([2H])cc45)c([2H])c3[2H])c([2H])c([2H])c2-c2cc([2H])c([2H])c([2H])c2[2H])c([2H])c([2H])c1[2H]]

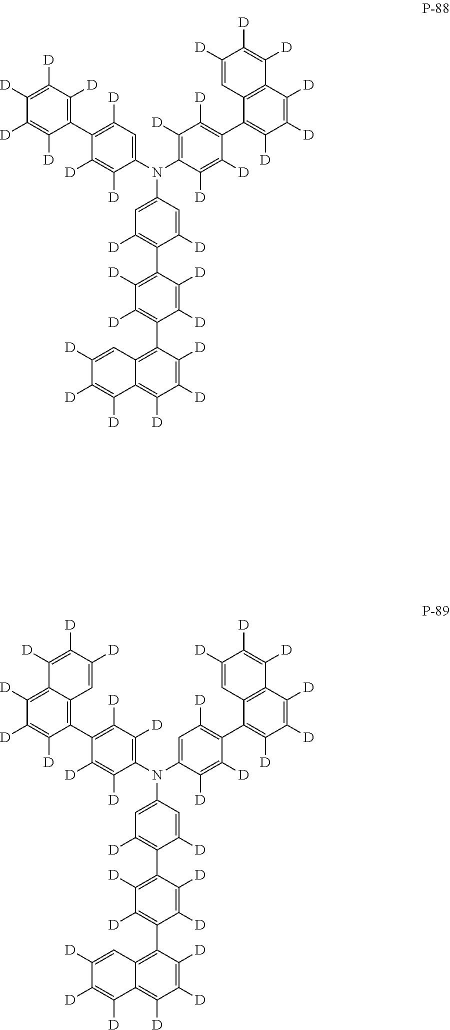

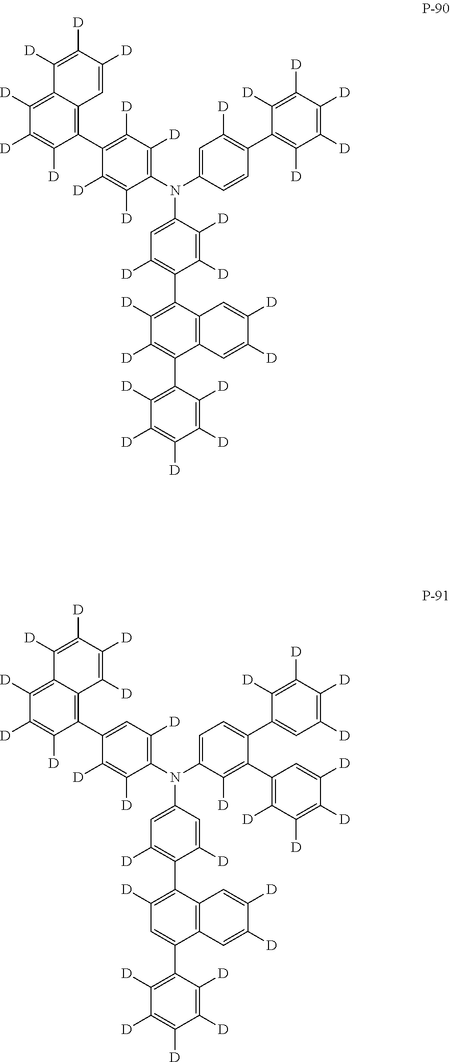 [SMILES: [2H]c1cc2c(-c3c([2H])cc(N(c4ccc(-c5c([2H])c([2H])c([2H])c([2H])c5[2H])c([2H])c4)c4c([2H])c([2H])c(-c5c([2H])c([2H])c([2H])c6c([2H])c([2H])c([2H])cc56)c([2H])c4[2H])c([2H])c3[2H])c([2H])c([2H])c(-c3c([2H])c([2H])c([2H])c([2H])c3[2H])c2cc1[2H] ; [2H]c1cc2c(-c3c([2H])c([2H])c([2H])c([2H])c3[2H])cc([2H])c(-c3c([2H])cc(N(c4ccc(-c5cc([2H])c([2H])c([2H])c5[2H])c(-c5cc([2H])c([2H])c([2H])c5[2H])c4[2H])c4c([2H])cc(-c5c([2H])c([2H])c([2H])c6c([2H])c([2H])c([2H])c([2H])c56)c([2H])c4[2H])cc3[2H])c2cc1[2H]]

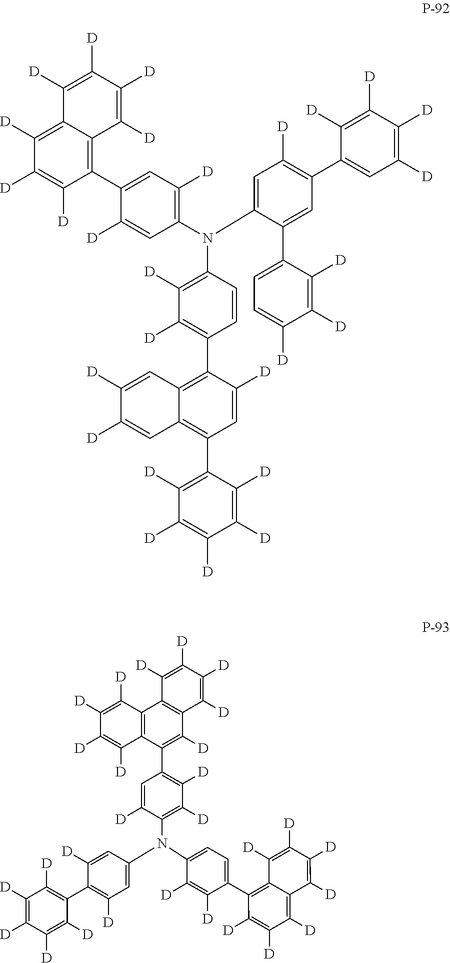 [SMILES: [2H]c1cc2c(-c3c([2H])c([2H])c([2H])c([2H])c3[2H])cc([2H])c(-c3ccc(N(c4cc([2H])c(-c5c([2H])c([2H])c([2H])c6c([2H])c([2H])c([2H])c([2H])c56)cc4[2H])c4cc([2H])c(-c5cc([2H])c([2H])c([2H])c5[2H])cc4-c4ccc([2H])c([2H])c4[2H])c([2H])c3[2H])c2cc1[2H] ; [2H]c1cc(N(c2ccc(-c3c([2H])c([2H])c([2H])c4c([2H])c([2H])c([2H])c([2H])c34)c([2H])c2[2H])c2c([2H])cc(-c3c([2H])c4c([2H])c([2H])c([2H])c([2H])c4c4c([2H])c([2H])c([2H])c([2H])c34)c([2H])c2[2H])cc([2H])c1-c1c([2H])c([2H])c([2H])c([2H])c1[2H]]

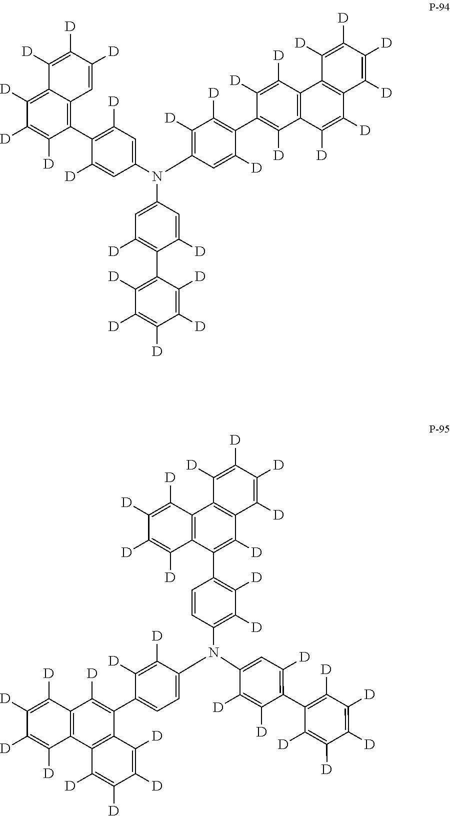 [SMILES: [2H]c1cc2c(-c3c([2H])cc(N(c4cc([2H])c(-c5c([2H])c([2H])c([2H])c([2H])c5[2H])c([2H])c4)c4cc([2H])c(-c5c([2H])c([2H])c6c(c5[2H])c([2H])c([2H])c5c([2H])c([2H])c([2H])c([2H])c56)c([2H])c4[2H])cc3[2H])c([2H])c([2H])c([2H])c2c([2H])c1[2H] ; [2H]c1cc(N(c2ccc(-c3c([2H])c4c([2H])c([2H])c([2H])c([2H])c4c4c([2H])c([2H])c([2H])c([2H])c34)c([2H])c2[2H])c2ccc(-c3c([2H])c4c([2H])c([2H])c([2H])c([2H])c4c4c([2H])c([2H])c([2H])c([2H])c34)c([2H])c2[2H])c([2H])c([2H])c1-c1c([2H])c([2H])c([2H])c([2H])c1[2H]]

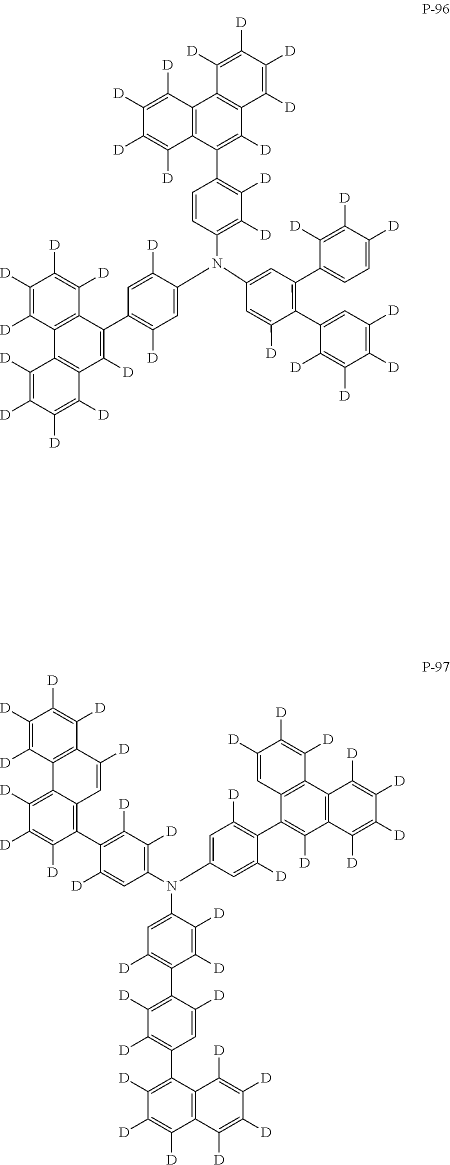

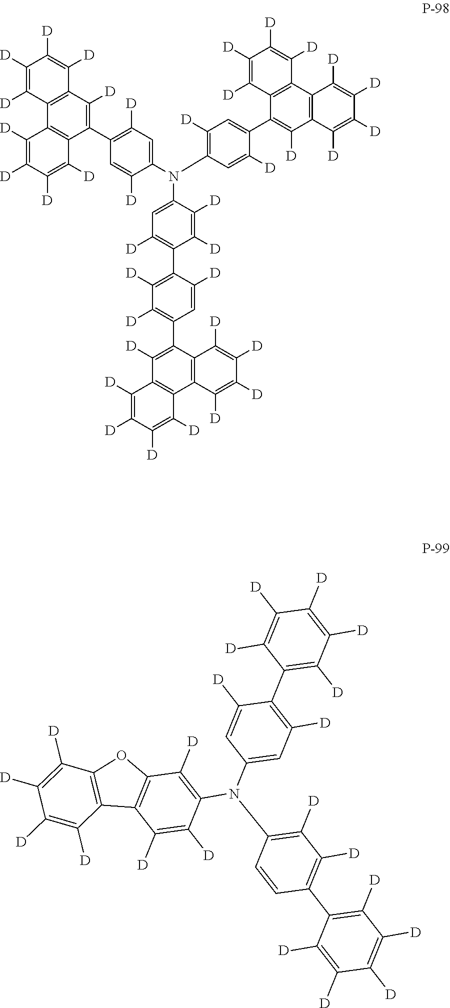

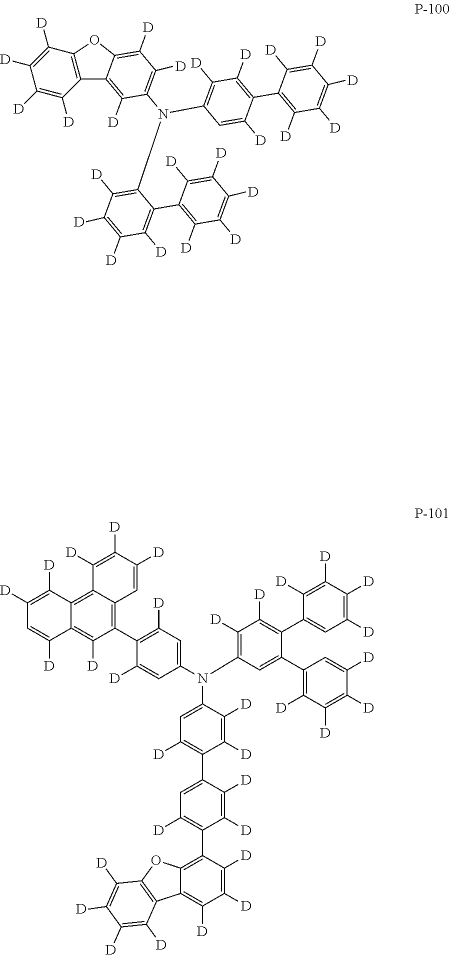 [SMILES: [2H]c1cc(N(c2c([2H])c([2H])c([2H])c([2H])c2-c2c([2H])c([2H])c([2H])c([2H])c2[2H])c2c([2H])c([2H])c3oc4c([2H])c([2H])c([2H])c([2H])c4c3c2[2H])c([2H])c([2H])c1-c1c([2H])c([2H])c([2H])c([2H])c1[2H] ; [2H]c1cc(-c2cc(N(c3cc([2H])c(-c4c([2H])c5c([2H])cc([2H])c([2H])c5c5c([2H])c([2H])c([2H])cc45)c([2H])c3)c3cc([2H])c(-c4cc([2H])c(-c5c([2H])c([2H])c([2H])c6c5oc5c([2H])c([2H])c([2H])c([2H])c56)c([2H])c4[2H])c([2H])c3[2H])c([2H])c([2H])c2-c2cc([2H])c([2H])c([2H])c2[2H])c([2H])c([2H])c1[2H]]

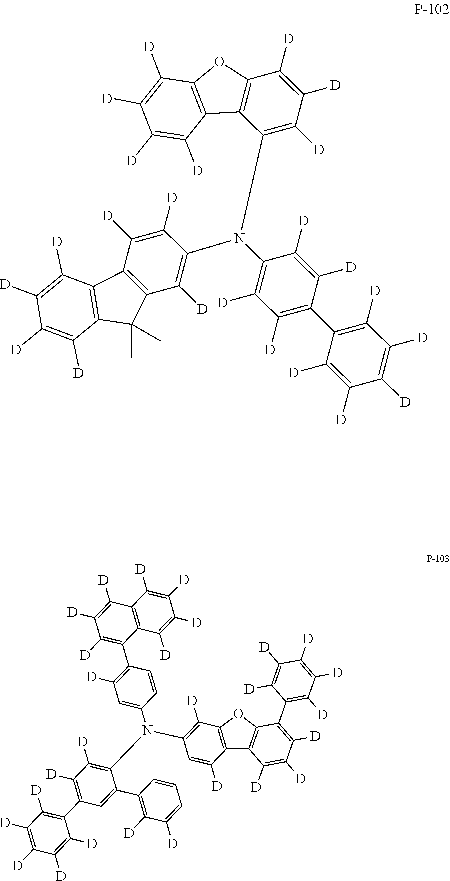 [SMILES: [2H]c1c([2H])c([2H])c(-c2c([2H])c([2H])c(N(c3c([2H])c([2H])c4c(c3[2H])C(C)(C)c3c([2H])c([2H])c([2H])c([2H])c3-4)c3c([2H])c([2H])c([2H])c4oc5c([2H])c([2H])c([2H])c([2H])c5c34)c([2H])c2[2H])c([2H])c1[2H] ; [2H]c1cc(N(c2cc([2H])c3c(oc4c(-c5c([2H])c([2H])c([2H])c([2H])c5[2H])c([2H])c([2H])c([2H])c43)c2[2H])c2c(-c3cccc([2H])c3[2H])cc(-c3c([2H])c([2H])c([2H])c([2H])c3[2H])c([2H])c2[2H])ccc1-c1c([2H])c([2H])c([2H])c2c([2H])c([2H])c([2H])c([2H])c12]

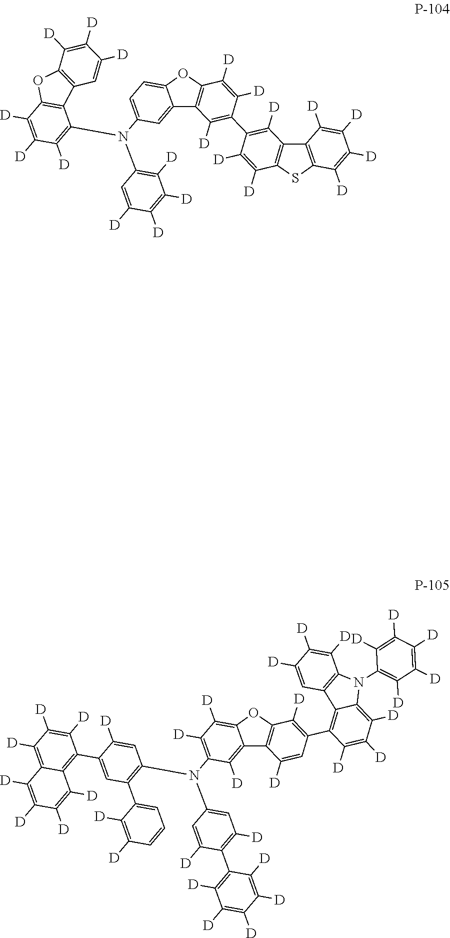 [SMILES: [2H]c1cc(N(c2ccc3oc4c([2H])c([2H])c(-c5c([2H])c([2H])c6sc7c([2H])c([2H])c([2H])c([2H])c7c6c5[2H])c([2H])c4c3c2)c2c([2H])c([2H])c([2H])c3oc4c([2H])c([2H])c([2H])cc4c23)c([2H])c([2H])c1[2H] ; [2H]c1cc(N(c2cc([2H])c(-c3c([2H])c([2H])c([2H])c([2H])c3[2H])c([2H])c2)c2c([2H])c([2H])c3oc4c([2H])c(-c5c([2H])c([2H])c([2H])c6c5c5cc([2H])c([2H])c([2H])c5n6-c5c([2H])c([2H])c([2H])c([2H])c5[2H])cc([2H])c4c3c2[2H])c(-c2cccc([2H])c2[2H])cc1-c1c([2H])c([2H])c([2H])c2c([2H])c([2H])c([2H])c([2H])c12]

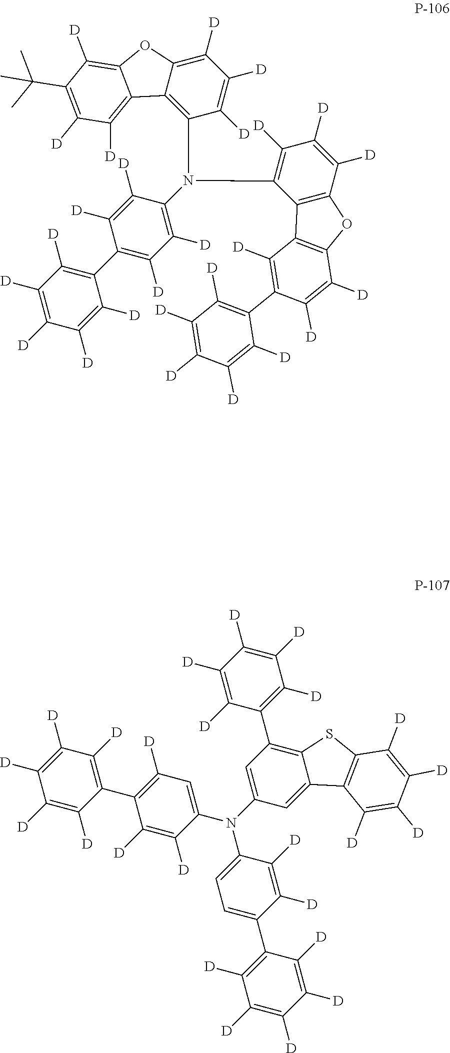 [SMILES: [2H]c1c([2H])c([2H])c(-c2c([2H])c([2H])c(N(c3c([2H])c([2H])c([2H])c4oc5c([2H])c(C(C)(C)C)c([2H])c([2H])c5c34)c3c([2H])c([2H])c([2H])c4oc5c([2H])c([2H])c(-c6c([2H])c([2H])c([2H])c([2H])c6[2H])c([2H])c5c34)c([2H])c2[2H])c([2H])c1[2H] ; [2H]c1cc(N(c2cc(-c3c([2H])c([2H])c([2H])c([2H])c3[2H])c3sc4c([2H])c([2H])c([2H])c([2H])c4c3c2)c2ccc(-c3c([2H])c([2H])c([2H])c([2H])c3[2H])c([2H])c2[2H])c([2H])c([2H])c1-c1c([2H])c([2H])c([2H])c([2H])c1[2H]]

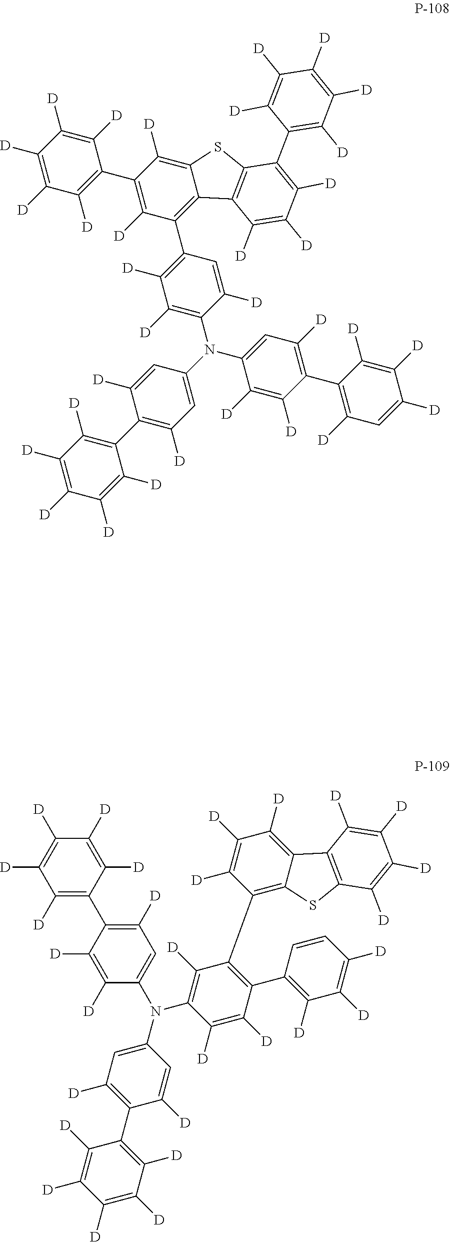

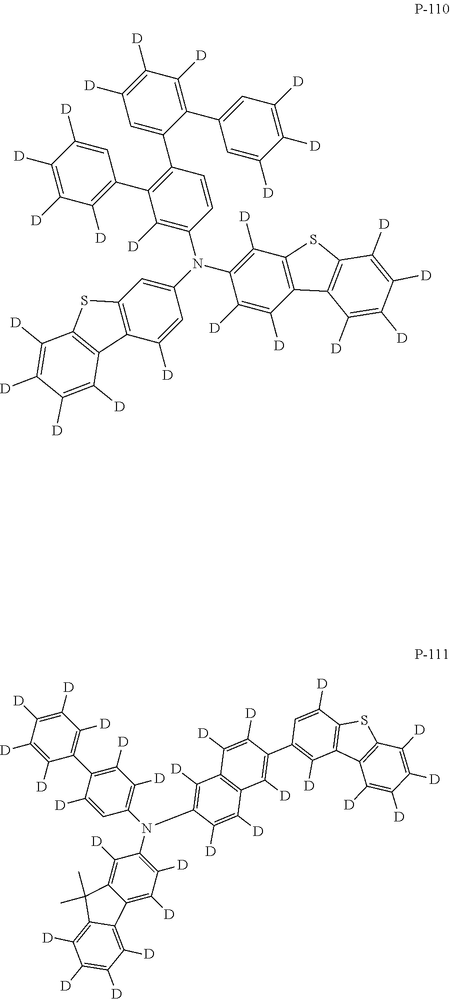 [SMILES: [2H]c1cc(-c2c(-c3ccc(N(c4cc([2H])c5c(c4)sc4c([2H])c([2H])c([2H])c([2H])c45)c4c([2H])c([2H])c5c(sc6c([2H])c([2H])c([2H])c([2H])c65)c4[2H])c([2H])c3-c3cc([2H])c([2H])c([2H])c3[2H])cc([2H])c([2H])c2[2H])cc([2H])c1[2H] ; [2H]c1cc(N(c2c([2H])c([2H])c3c(c2[2H])C(C)(C)c2c([2H])c([2H])c([2H])c([2H])c2-3)c2c([2H])c([2H])c3c([2H])c(-c4cc([2H])c5sc6c([2H])c([2H])c([2H])c([2H])c6c5c4[2H])c([2H])c([2H])c3c2[2H])c([2H])c([2H])c1-c1c([2H])c([2H])c([2H])c([2H])c1[2H]]

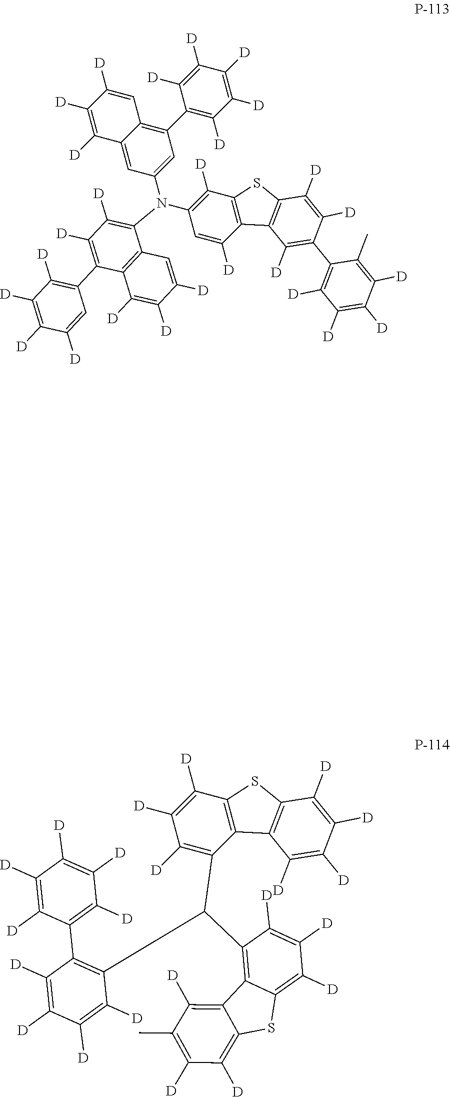 [SMILES: [2H]c1cc(-c2c([2H])c([2H])c(N(c3cc(-c4c([2H])c([2H])c([2H])c([2H])c4[2H])c4cc([2H])c([2H])c([2H])c4c3)c3cc([2H])c4c(sc5c([2H])c([2H])c(-c6c([2H])c([2H])c([2H])c([2H])c6C)c([2H])c54)c3[2H])c3cc([2H])c([2H])c([2H])c23)c([2H])c([2H])c1[2H] ; [2H]c1c([2H])c([2H])c(-c2c([2H])c([2H])c([2H])c([2H])c2C(c2c([2H])c([2H])c([2H])c3sc4c([2H])c([2H])c([2H])c([2H])c4c23)c2c([2H])c([2H])c([2H])c3sc4c([2H])c([2H])c(C)c([2H])c4c23)c([2H])c1[2H]]

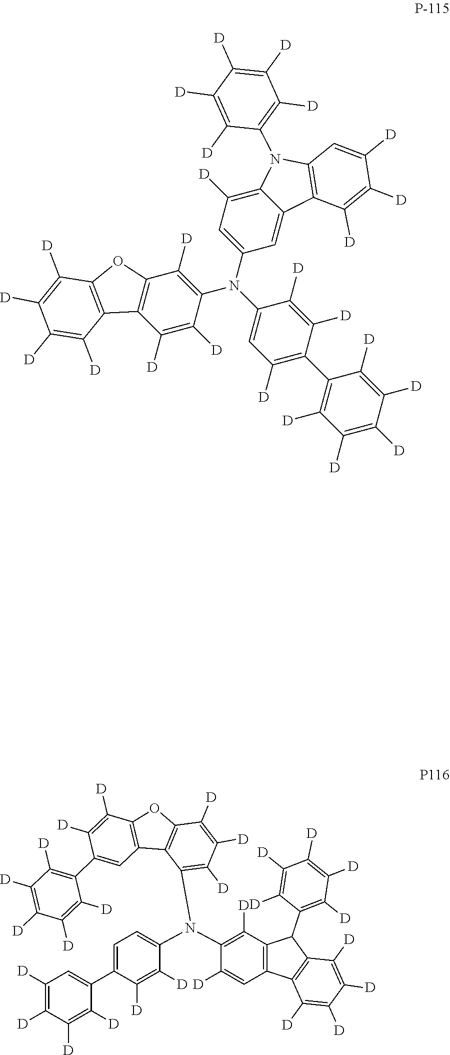 [SMILES: [2H]c1cc2c(c([2H])c1[2H])c1cc(N(c3cc([2H])c(-c4c([2H])c([2H])c([2H])c([2H])c4[2H])c([2H])c3[2H])c3c([2H])c([2H])c4c(oc5c([2H])c([2H])c([2H])c([2H])c54)c3[2H])cc([2H])c1n2-c1c([2H])c([2H])c([2H])c([2H])c1[2H] ; [2H]c1cc(-c2ccc(N(c3c([2H])cc4c(c3[2H])C(c3c([2H])c([2H])c([2H])c([2H])c3[2H])c3c([2H])c([2H])c([2H])c([2H])c3-4)c3c([2H])c([2H])c([2H])c4oc5c([2H])c([2H])c(-c6c([2H])c([2H])c([2H])c([2H])c6[2H])cc5c34)c([2H])c2[2H])c([2H])c([2H])c1[2H]]

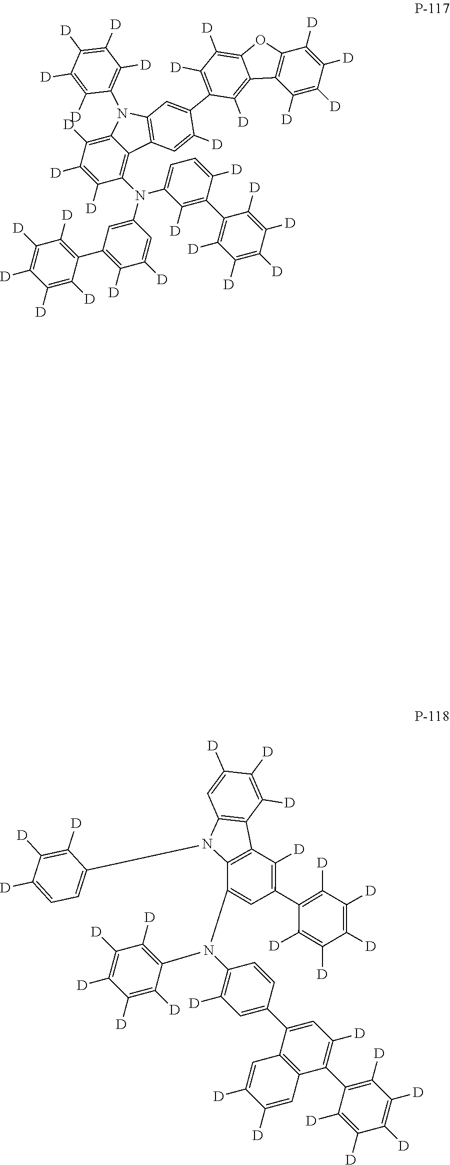 [SMILES: [2H]c1cc2c3c(N(c4cc([2H])c([2H])c(-c5c([2H])c([2H])c([2H])c([2H])c5[2H])c4)c4ccc([2H])c(-c5c([2H])c([2H])c([2H])c([2H])c5[2H])c4[2H])c([2H])c([2H])c([2H])c3n(-c3c([2H])c([2H])c([2H])c([2H])c3[2H])c2cc1-c1c([2H])c([2H])c2oc3c([2H])c([2H])c([2H])c([2H])c3c2c1[2H] ; [2H]c1cc2c(-c3ccc(N(c4c([2H])c([2H])c([2H])c([2H])c4[2H])c4cc(-c5c([2H])c([2H])c([2H])c([2H])c5[2H])c([2H])c5c6c([2H])c([2H])c([2H])cc6n(-c6ccc([2H])c([2H])c6[2H])c45)c([2H])c3)cc([2H])c(-c3c([2H])c([2H])c([2H])c([2H])c3[2H])c2cc1[2H]]

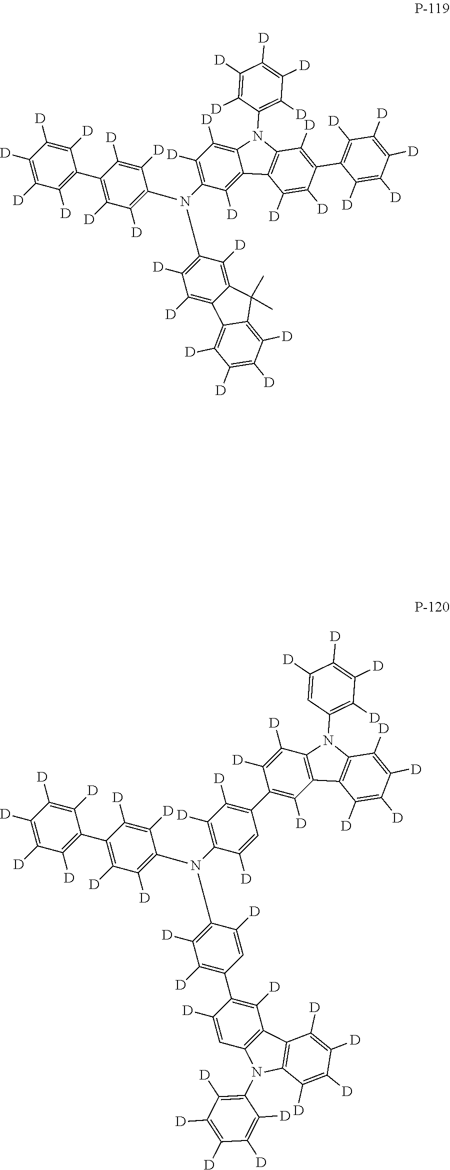

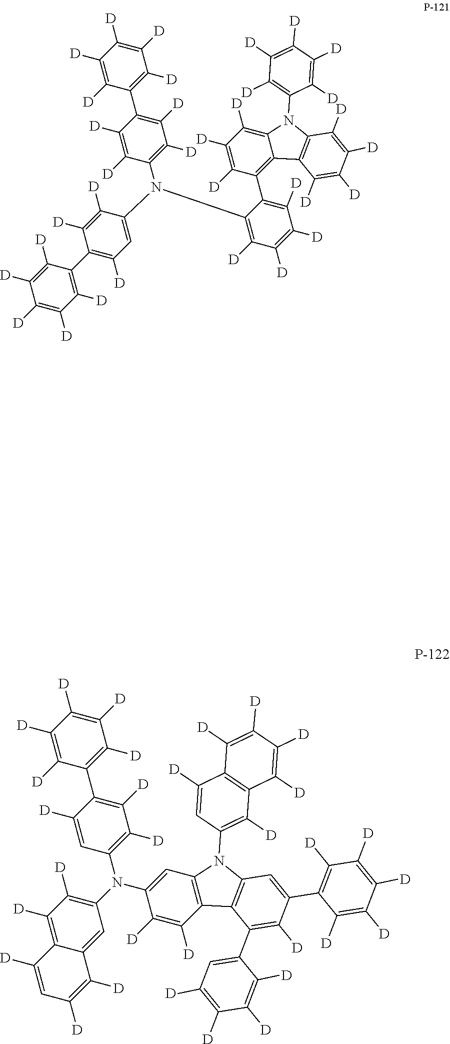

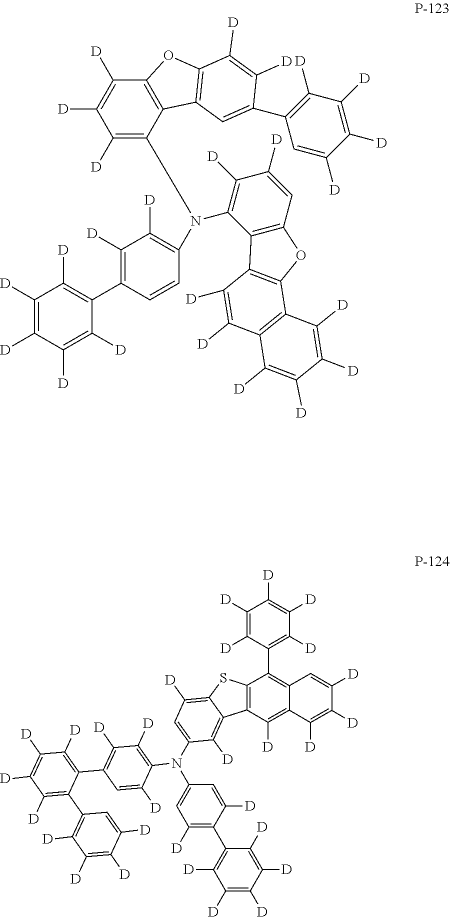 [SMILES: [2H]c1cc(-c2cc3c(oc4c([2H])c([2H])c([2H])c(N(c5ccc(-c6c([2H])c([2H])c([2H])c([2H])c6[2H])c([2H])c5[2H])c5c([2H])c([2H])cc6oc7c8c([2H])c([2H])c([2H])c([2H])c8c([2H])c([2H])c7c56)c43)c([2H])c2[2H])c([2H])c([2H])c1[2H] ; [2H]c1cc(-c2c([2H])c([2H])c([2H])c([2H])c2-c2cc([2H])c(N(c3cc([2H])c(-c4c([2H])c([2H])c([2H])c([2H])c4[2H])c([2H])c3)c3cc([2H])c4sc5c(-c6c([2H])c([2H])c([2H])c([2H])c6[2H])c6cc([2H])c([2H])c([2H])c6c([2H])c5c4c3[2H])c([2H])c2[2H])c([2H])c([2H])c1[2H]]

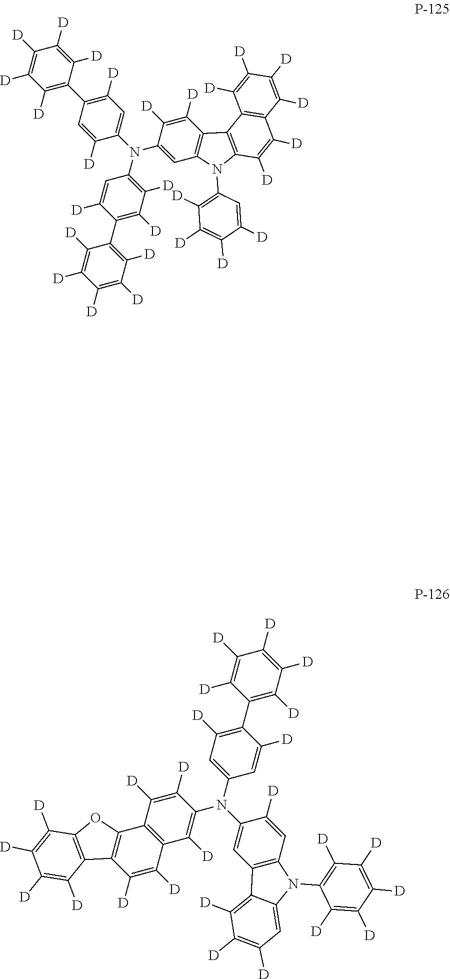 [SMILES: [2H]c1cc(N(c2cc([2H])c(-c3c([2H])c([2H])c([2H])c([2H])c3[2H])c([2H])c2[2H])c2cc3c(c([2H])c2[2H])c2c4c([2H])c([2H])c([2H])c([2H])c4c([2H])c([2H])c2n3-c2cc([2H])c([2H])c([2H])c2[2H])c([2H])cc1-c1c([2H])c([2H])c([2H])c([2H])c1[2H] ; [2H]c1cc2c(cc1N(c1cc([2H])c(-c3c([2H])c([2H])c([2H])c([2H])c3[2H])c([2H])c1)c1c([2H])c([2H])c3c(c1[2H])c([2H])c([2H])c1c3oc3c([2H])c([2H])c([2H])c([2H])c31)c1c([2H])c([2H])c([2H])cc1n2-c1c([2H])c([2H])c([2H])c([2H])c1[2H]]

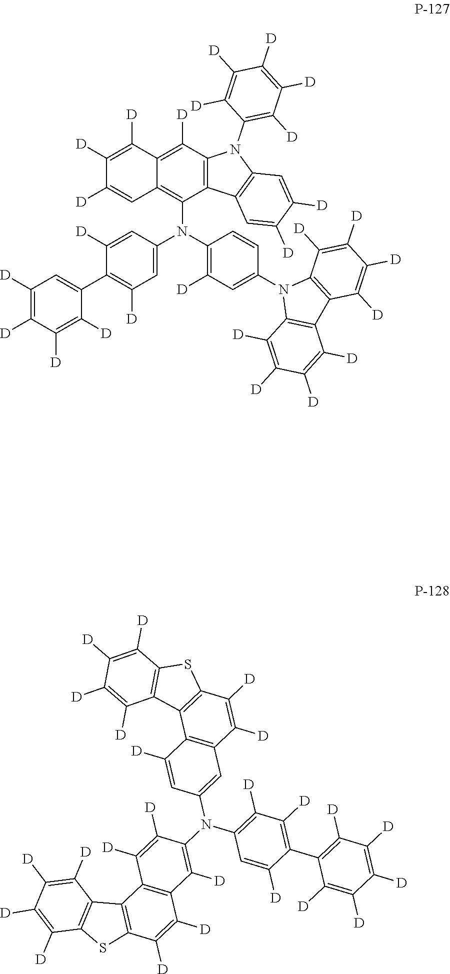 [SMILES: [2H]c1cc2c3c(N(c4cc([2H])c(-c5cc([2H])c([2H])c([2H])c5[2H])c([2H])c4)c4ccc(-n5c6c([2H])c([2H])c([2H])c([2H])c6c6c([2H])c([2H])c([2H])c([2H])c65)cc4[2H])c4cc([2H])c([2H])c([2H])c4c([2H])c3n(-c3c([2H])c([2H])c([2H])c([2H])c3[2H])c2cc1[2H] ; [2H]c1cc(N(c2cc([2H])c3c(c2)c([2H])c([2H])c2sc4c([2H])c([2H])c([2H])c([2H])c4c23)c2c([2H])c([2H])c3c(c2[2H])c([2H])c([2H])c2sc4c([2H])c([2H])c([2H])c([2H])c4c23)c([2H])c([2H])c1-c1c([2H])c([2H])c([2H])c([2H])c1[2H]]

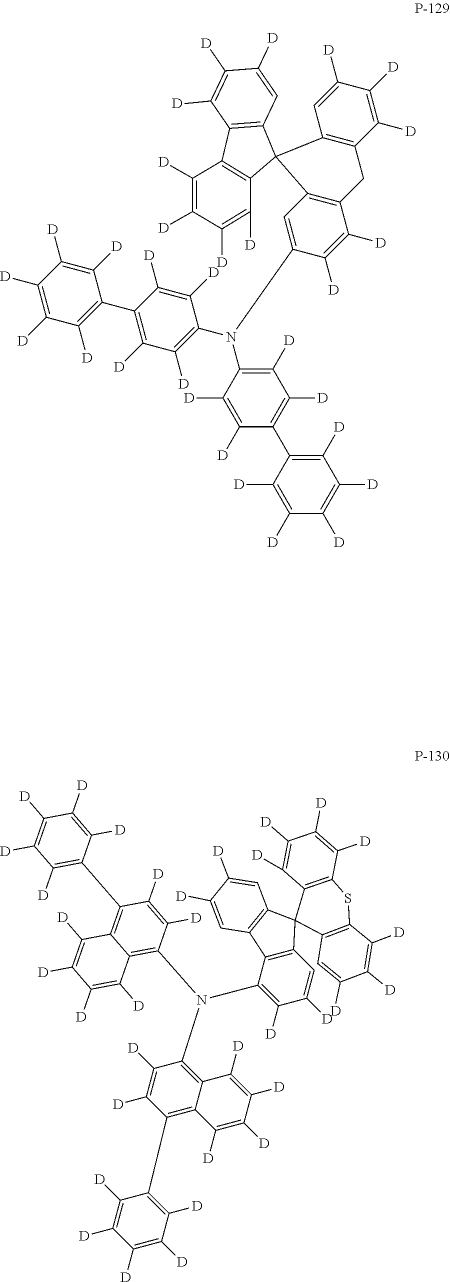

Also, the present invention provides a method for preparing the deuterated compound represented by Formula (A) comprising:
(a) a step of forming a first reactant by dissolving the compound represented by Formula A in perdeuterated benzene (benzene-D₆);
(b) a step of forming a second reactant by adding deuterium-triflic acid (CF₃SO₃D) to the first reactant;
(c) a step deuterated by reacting the second reactant at 80° C. for 3 hours to 18 hours;
(d) a step quenched by adding Na₂CO₃ in D₂O, after the reaction is completed, the second reactant is cooled to room temperature,
(e) a step to obtain the deuterated compound represented by Formula A after concentrating the organic solvent of the second reactant, recrystallization with toluene and acetone solvent.
In step (c), the deuterated reaction time may be 3 hours to 18 hours, and preferably performed for 3 hours.

Also, the present invention provides a method for preparing a deuterated compound represented by Formula A in which the compound represented by Formula A is synthesized by reacting a compound represented by Formula R1 or Formula R2 and a compound represented by Formula R3.

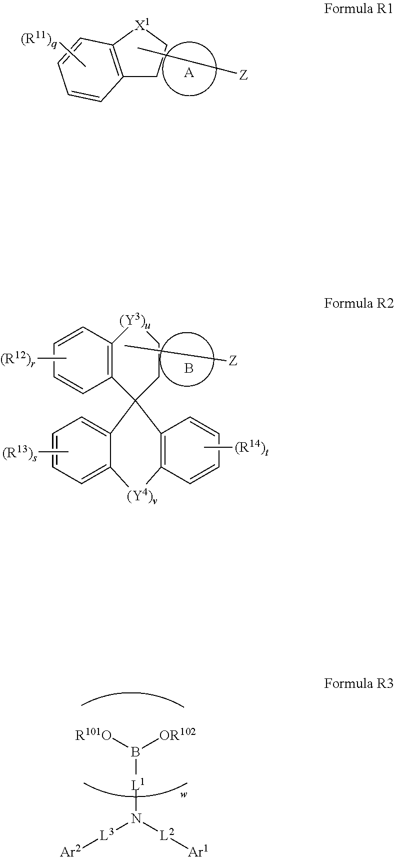

Wherein, each symbol may be defined as follows.

1) L¹, L², L³, Ar¹, Ar², R¹¹, R¹², R¹³, R¹⁴, ring A, ring B, q, r, s, t, u and v are the same as above,

2) R¹⁰¹ and R¹⁰² are each independently selected from the group consisting of a C₁-C₅₀ alkyl group; a C₂-C₂₀ alkenyl group; a C₂-C₂₀ alkynyl group; a C₁-C₃₀ alkoxyl group; and a C₆-C₃₀ aryloxy group; or R¹⁰¹ and R¹⁰² are bonded to adjacent groups to form a ring.

Wherein in case R¹⁰¹ and R¹⁰² are an alkyl group, they may be preferably a C₁-C₃₀ alkyl group, more preferably a C₁-C₂₄ alkyl group.

Wherein in case R¹⁰¹ and R¹⁰² are an alkoxyl group, they may be preferably an C₁˜C₂₄ alkoxyl group.

Wherein in case R¹⁰¹ and R¹⁰² are an aryloxy group, they may be preferably an C₆˜C₂₄ aryloxy group.

3) Z is halogen; or a triflate group;

4) w is 0 or 1.

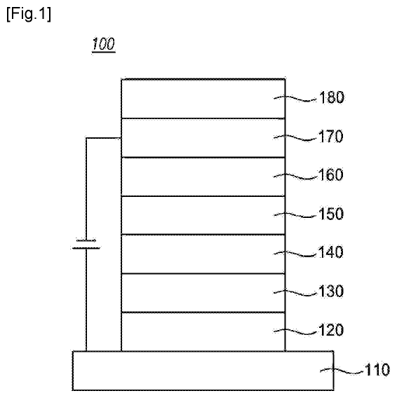

Referring to FIG. 1, the organic electric element (100) according to the present invention includes a first electrode (110), a second electrode (170), and an organic material layer including a single compound or 2 or more compounds represented by Formula A between the first electrode (110) and the second electrode (170). In this case, the first electrode (110) may be an anode, and the second electrode (170) may be a cathode. In the case of an inverted type, the first electrode may be a cathode and the second electrode may be an anode.

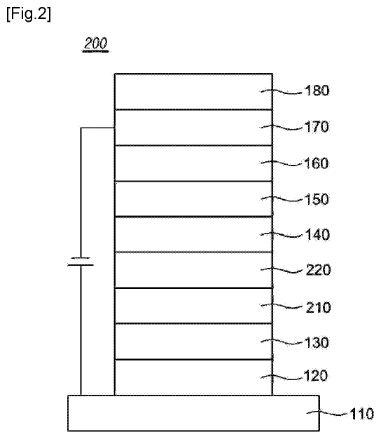

The organic material layer may sequentially include a hole injection layer (120), a hole transport layer (130), an emitting layer (140), an electron transport layer (150), and an electron injection layer (160) on the first electrode (110). In this case, the remaining layers except for the emitting layer (140) may not be formed. It may further include a hole blocking layer, an electron blocking layer, an emitting-auxiliary layer (220), a buffer layer (210), etc. and the electron transport layer (150) and the like may serve as a hole blocking layer. (See FIG. 2) Also, the organic electric element according to an embodiment of the present invention may further include a protective layer or a light efficiency enhancing layer (180). The light efficiency enhancing layer may be formed on one of both surfaces of the first electrode not in contact with the organic material layer or on one of both surfaces of the second electrode not in contact with the organic material layer. The compound according to an embodiment of the present invention applied to the organic material layer may be used as the hole injection layer (120), the hole transport layer (130), the emitting-auxiliary layer (220), electron transport auxiliary layer, the electron transport layer (150), and an electron injection layer (160), a host or dopant of the emitting layer (140) or a material for the light efficiency enhancing layer. Preferably, for example, the compound according to Formula A of the present invention may be used as a material for an emitting auxiliary layer or a hole transport layer.

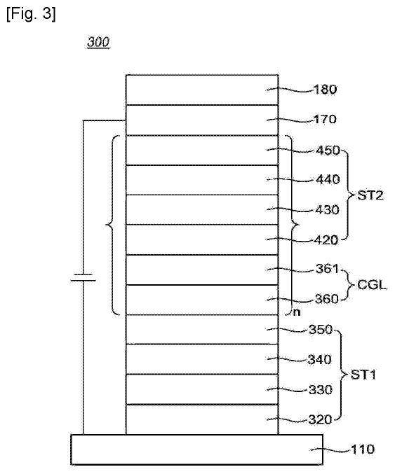

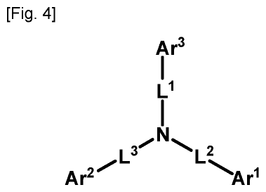

The organic material layer may include 2 or more stacks including a hole transport layer, an emitting layer and an electron transport layer sequentially formed on the anode, further include a charge generation layer formed between the 2 or more stacks (see FIG. 3).

Otherwise, even with the same core, the band gap, electrical characteristics, interface characteristics, etc. may vary depending on which position the substituent is bonded to, therefore the choice of core and the combination of sub-substituents bound thereto are also very important, and in particular, when the optimal combination of energy levels and T1 values and unique properties of materials (mobility, interfacial characteristics, etc.) of each organic material layer is achieved, a long lifespan and high efficiency can be achieved at the same time.

The organic electroluminescent device according to an embodiment of the present invention may be manufactured using a PVD (physical vapor deposition) method. For example, depositing a metal or a metal oxide having conductivity or an alloy thereof on a substrate to form an anode, and after forming an organic material layer including the hole injection layer (120), the hole transport layer (130), the emitting layer (140), the electron transport layer (150) and the electron injection layer (160) thereon, it can be prepared by depositing a material that can be used as a cathode thereon.

Also, in the present invention, the organic material layer is formed by any one of a spin coating process, a nozzle printing process, an inkjet printing process, a slot coating process, a dip coating process, and a roll-to-roll process, and the organic material layer provides an organic electric element comprising the compound as an electron transport material.

As another specific example, the compound of the same or different types of the compound represented by Formula A is mixed and used in the organic material layer. Also, the present invention provides an emitting auxiliary layer composition comprising the compound represented by Formula A, and provides an organic electric element including the emitting auxiliary layer.

Also, the present invention provides a hole transport layer composition comprising the compound represented by Formula A, and provides an organic electric element including the hole transport layer.

Also, the present invention provides an electronic device comprising a display device including the organic electric element; and a control unit for driving the display device; In another aspect, the organic electric element is at least one of an organic electroluminescent device, an organic solar cell, an organic photo conductor, an organic transistor, and a device for monochromatic or white lighting. At this time, the electronic device may be a current or future wired/wireless communication terminal, and covers all kinds of electronic devices including mobile communication terminals such as mobile phones, a personal digital assistant(PDA), an electronic dictionary, a point-to-multipoint(PMP), a remote controller, a navigation unit, a game player, various kinds of TVs, and various kinds of computers.

Hereinafter, a synthesis example of the compound represented by Formula A of the present invention and a manufacturing example of an organic electric element of the present invention will be described in detail with reference to Examples, but the present invention is not limited to the following Examples.

### Synthesis Example 1

The compound (final product 1-1) represented by Formula 1 according to the present invention is prepared by reacting Sub 1 with Sub 2 as shown in Scheme 1.

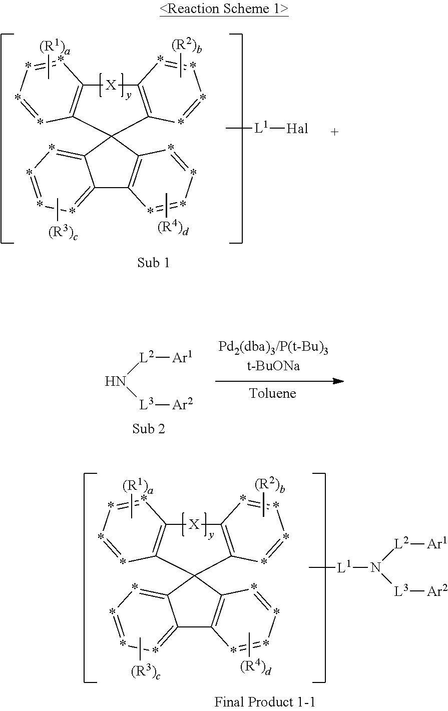

The deuterated compound of compound (final product 1-1) represented by Formula 1 according to the present invention is prepared by dissolving in perdeuterated benzene (benzene-D₆), adding deuterium-triflic acid (CF₃SO₃D), and reacting at a temperature of 80° C. for 3 hours to 18 hours, and more preferably 3 hours.

### 1. Synthesis Example of P-1

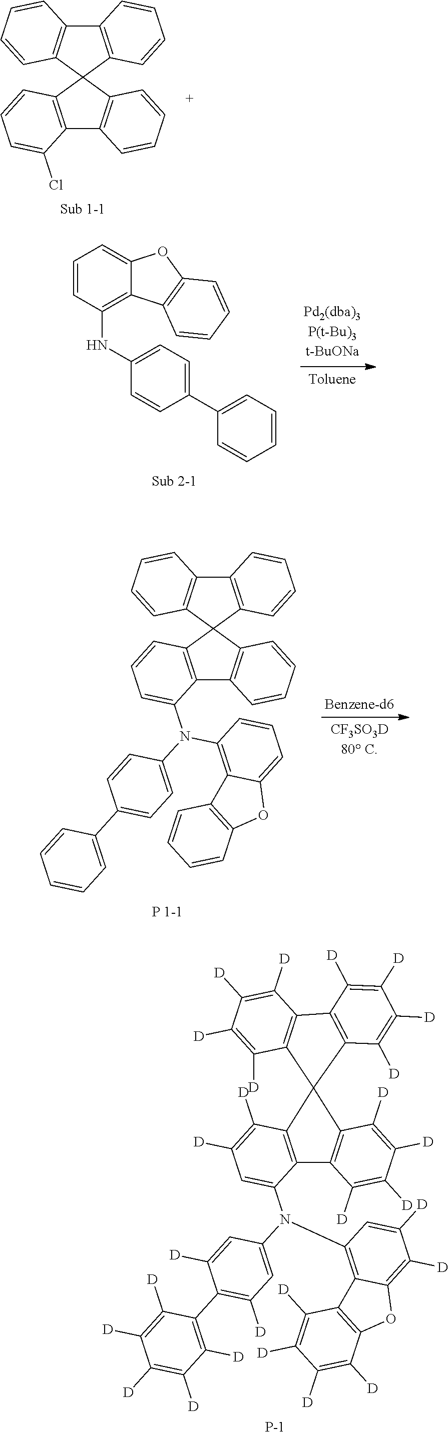

### (1) Synthesis of P 1-1

Sub 1-1 (7.8 g, 22.2 mmol), Sub 2-1 (7.5 g, 22.2 mmol), Pd₂(dba)₃ (0.6 g, 0.7 mmol), t-BuONa (4.3 g, 44.5 mmol) were placed in a round-bottom flask and dissolved in anhydrous Toluene (130 mL), then P(t-Bu)₃ (50 wt % Sol.) (0.54 mL, 1.3 mmol) was added and heated and stirred at 110° C. for about 4 hours. After confirming the completion of the reaction by TLC, cooled to room temperature and extracted with CH₂Cl₂ and water. The separated organic layer was dried over MgSO₄, concentrated, and the resulting compound was recrystallized using silicagel column (Hexane:CH₂Cl₂=4:1) to obtain 10.8 g (yield: 75%) of P 1-1.

### (2) Synthesis of P-1

P 1-1 (10.8 g, 16.6 mmol) obtained in the above synthesis was dissolved in perdeuterated benzene (C₆D₆) (126.0 g, 1,495.9 mmol) and CF₃SO₃D (12.5 g, 83.1 mmol) was added thereto, followed by a temperature of 80° C. reacted for 3 hours to form a deuterated material. Periodically take a sample, measure the degree of deuterium by LC-MS, and after the deuterium exchange reaction is completed at a desired substitution rate, cool to room temperature, quench by adding Na₂CO₃ in D₂O, and concentrate the organic solvent. Recrystallization using toluene and acetone solvent gave 10.3 g (yield: 92%) of deuterated compound P-1. The final mass was determined by LC-MS to confirm to be 87.1% deuterated.

### 2. Synthesis Example of P-2

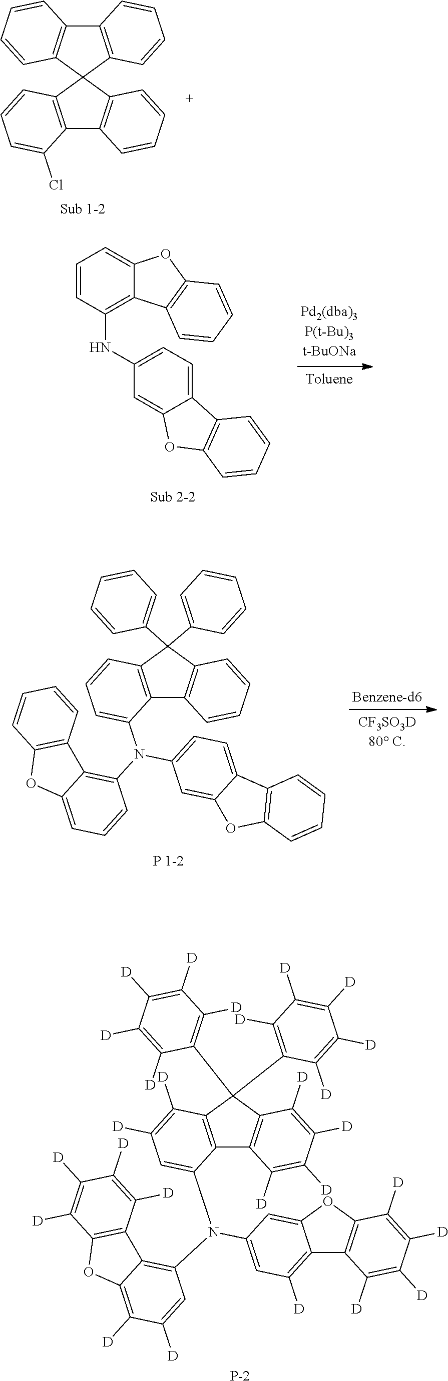

### (1) Synthesis of P 1-2

Sub 1-2 (9.0 g, 25.5 mmol), Sub 2-2 (8.9 g, 25.5 mmol), Pd₂(dba)₃ (0.7 g, 0.8 mmol), t-BuONa (4.9 g, 51.0 mmol) were placed in a round-bottom flask and dissolved in anhydrous Toluene (150 mL), then P(t-Bu)₃ (50 wt % Sol.) (0.62 mL, 1.5 mmol) was added and heated and stirred at 110° C. for about 4 hours. After confirming the completion of the reaction by TLC, cooled to room temperature and extracted with CH₂Cl₂ and water. The separated organic layer was dried over MgSO₄, concentrated, and the resulting compound was recrystallized using silicagel column (Hexane:CH₂Cl₂=4:1) to obtain 13.6 g (yield: 80%) of P 1-2.

### (2) Synthesis of P-2

P 1-2 (13.6 g, 20.4 mmol) obtained in the above synthesis was dissolved in perdeuterated benzene (C₆D₆) (154.7 g, 1,838.4 mmol) and CF₃SO₃D (15.3 g, 102.1 mmol) was added thereto, followed by reacting a temperature at 80° C. for 3 hours to form a deuterated material. Periodically take a sample, measure the degree of deuteration by LC-MS, and after the deuterium exchange reaction is completed at the desired substitution rate, cool to room temperature, quench by adding Na₂CO₃ in D₂O, and the organic solvent is concentrated. Recrystallization using toluene and acetone solvent gave 13.3 g (yield: 94%) of deuterated compound P-2. The final mass was determined by LC-MS to confirm to be 87.1% deuterated.

### 3. Synthesis Example of P-3

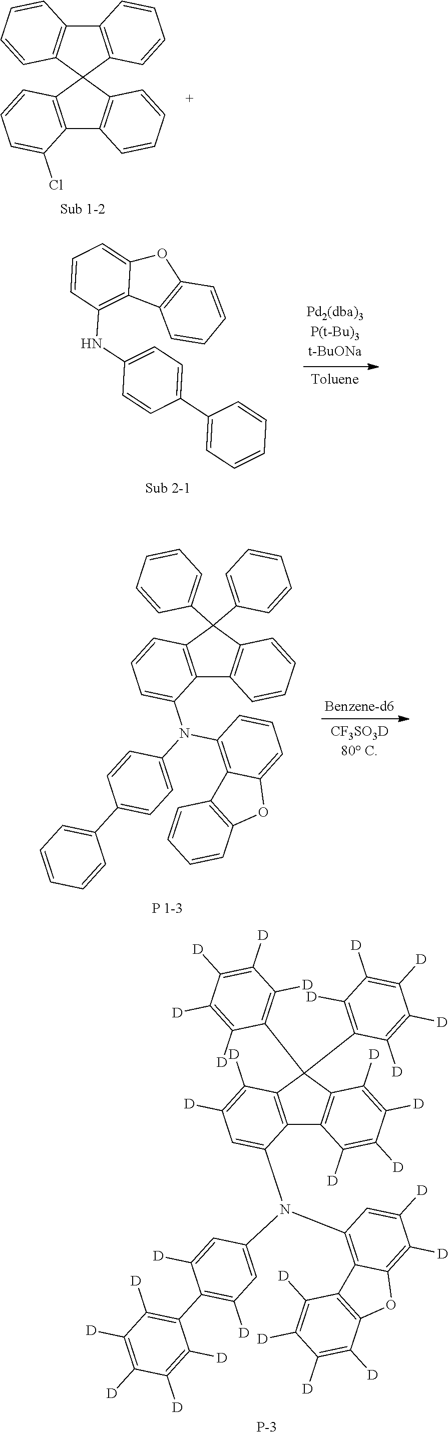

### (1) Synthesis of P 1-3

Sub 1-2 (7.8 g, 22.1 mmol), Sub 2-1 (7.4 g, 22.1 mmol), Pd₂(dba)₃ (0.6 g, 0.7 mmol), t-BuONa (4.3 g, 44.2 mmol) were placed in a round-bottom flask and dissolved in anhydrous Toluene (130 mL), then P(t-Bu)₃ (50 wt % Sol.) (0.54 mL, 1.3 mmol) was added and heated and stirred at 110° C. for about 4 hours. After confirming the completion of the reaction by TLC, cooled to room temperature and extracted with CH₂Cl₂ and water. The separated organic layer was dried over MgSO₄, concentrated, and the resulting compound was recrystallized using silicagel column (Hexane:CH₂Cl₂=4:1) to obtain 11.4 g (yield: 79%) of P 1-3.

### (2) Synthesis of P-3

P 1-3 (11.4 g, 17.5 mmol) obtained in the above synthesis was dissolved in perdeuterated benzene (C₆D₆) (132.5 g, 1,574.1 mmol) and CF₃SO₃D (13.1 g, 87.5 mmol) was added thereto, followed by reacting a temperature at 80° C. for 3 hours to form a deuterated material. Periodically take a sample, measure the degree of deuteration by LC-MS, and after the deuterium exchange reaction is completed at the desired substitution rate, cool to room temperature, quench by adding Na₂CO₃ in D₂O, and the organic solvent is concentrated. Recrystallization using toluene and acetone solvent gave 10.7 g (yield: 90%) of deuterated compound P-3. The final mass was determined by LC-MS to confirm to be 87.8% deuterated.

### 4. Synthesis Example of P-4

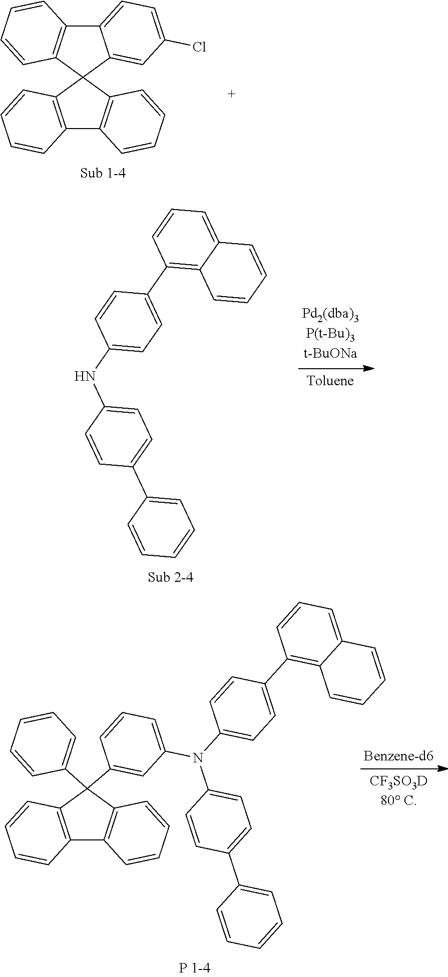 [SMILES: Clc1ccc2c(c1)C1(c3ccccc3-c3ccccc31)c1ccccc1-2 ; c1ccc(-c2ccc(Nc3ccc(-c4cccc5ccccc45)cc3)cc2)cc1 ; c1ccc(-c2ccc(N(c3ccc(-c4cccc5ccccc45)cc3)c3cccc(C4(c5ccccc5)c5ccccc5-c5ccccc54)c3)cc2)cc1]

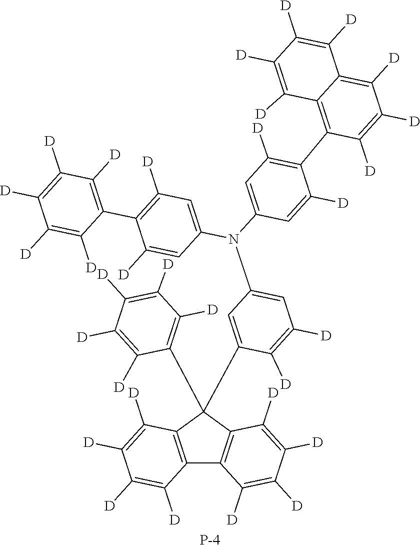

### (1) Synthesis of P 1-4

Sub 1-4 (9.6 g, 27.2 mmol), Sub 2-4 (10.1 g, 27.2 mmol), Pd₂(dba)₃ (0.8 g, 0.8 mmol), t-BuONa (5.2 g, 54.4 mmol) were placed in a round-bottom flask and dissolved in anhydrous Toluene (180 mL), then P(t-Bu)₃ (50 wt % Sol.) (0.66 mL, 1.6 mmol) was added and heated and stirred at 110° C. for about 4 hours. After confirming the completion of the reaction by TLC, cooled to room temperature and extracted with CH₂₁C₂ and water. The separated organic layer was dried over MgSO₄, concentrated, and the resulting compound was recrystallized using silicagel column (Hexane:CH₂Cl₂=4:1) to obtain 15.1 g (yield: 81%) of P 1-4.

### (2) Synthesis of P-4

P 1-4 (15.1 g, 22.1 mmol) obtained in the above synthesis was dissolved in perdeuterated benzene (C₆D₆) (167 g, 1,988.7 mmol) and CF₃SO₃D (16.6 g, 110.5 mmol) was added thereto, followed by reacting a temperature at 80° C. for 3 hours to form a deuterated material. Periodically take a sample, measure the degree of deuteration by LC-MS, and after the deuterium exchange reaction is completed at the desired substitution rate, cool to room temperature, quench by adding Na₂CO₃ in D₂O, and the organic solvent is concentrated. Recrystallization using toluene and acetone solvent gave 13.8 g (yield: 87%) of deuterated compound P-4. The final mass was determined by LC-MS to confirm to be 83.7% deuterated.

### Synthesis Example 2

The compound represented by Formula A according to the present invention (final product 2) is prepared by a reaction according to Reaction Scheme 2.

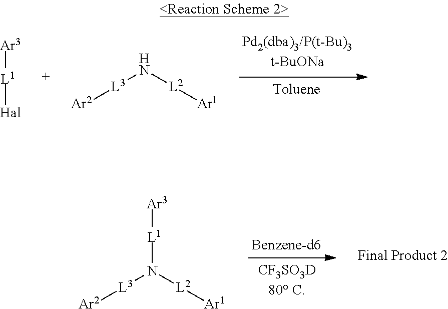

The deuterated compound of the compound represented by Formula A (final product 2) according to the present invention is dissolved in perdeuterated benzene (benzene-D₆), deuterium-triflic acid (CF₃SO₃D) is added, and prepared by reacting at the temperature of 80° C. for 3 hours to 18 hours, preferably 3 hours to 18 hours, and more preferably 3 hours.

### 1. Synthesis Example of P-84

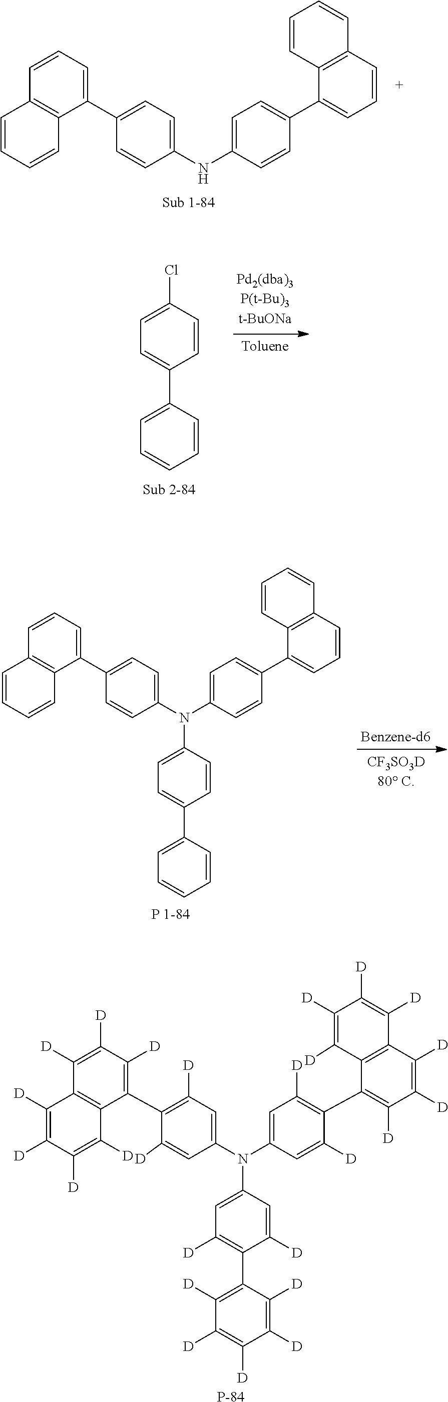

### (1) Synthesis of P 1-84

Sub 1-84 (10.0 g, 23.7 mmol), Sub 2-84 (4.3 g, 23.7 mmol), Pd₂(dba)₃ (0.7 g, 0.7 mmol), t-BuONa (4.6 g, 47.4 mmol) were placed in a round-bottom flask and dissolved in anhydrous Toluene (119 mL), then P(t-Bu)₃ (50 wt % Sol.) (0.6 mL, 1.4 mmol) was added and heated and stirred at 110° C. for about 4 hours. After confirming the completion of the reaction by TLC, cooled to room temperature and extracted with CH₂Cl₂ and water. The separated organic layer was dried over MgSO₄, concentrated, and the resulting compound was recrystallized using silicagel column (Hexane:CH₂Cl₂=4:1) to obtain 10.0 g (yield: 78%) of P 1-84.

### (2) Synthesis of P-84

P 1-84 (10.0 g, 18.5 mmol) obtained in the above synthesis was dissolved in perdeuterated benzene (C₆D₆) (132 g, 1,568.7 mmol) and CF₃SO₃D (13.1 g, 87.2 mmol) was added thereto, followed by reacting a temperature at 80° C.

Samples were taken periodically for 3 hours and the degree of deuteration was measured by LC-MS, after the deuterium exchange reaction is completed at the desired substitution rate, cool to room temperature, quench by adding Na₂CO₃ in D₂O, and the organic solvent is concentrated. Recrystallization using toluene and acetone solvent gave 9.7 g (yield: 93%) of deuterated compound P-72. The final mass was determined by LC-MS to confirm to be 80.6% deuterated.

### 2. Synthesis Example of P-106

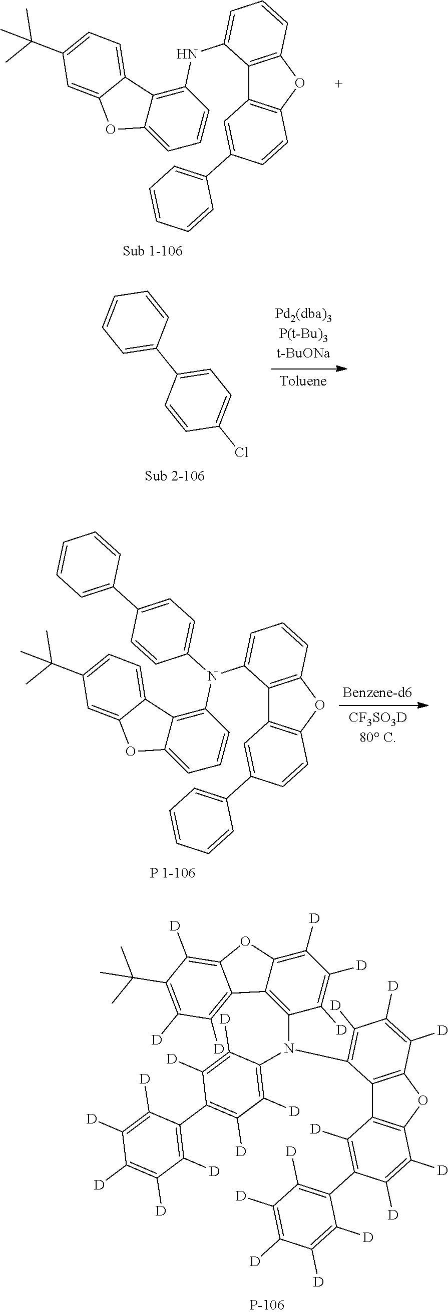 [SMILES: CC(C)(C)c1ccc2c(c1)oc1cccc(Nc3cccc4oc5ccc(-c6ccccc6)cc5c34)c12 ; Clc1ccc(-c2ccccc2)cc1 ; CC(C)(C)c1ccc2c(c1)oc1cccc(N(c3ccc(-c4ccccc4)cc3)c3cccc4oc5ccc(-c6ccccc6)cc5c34)c12 ; [2H]c1c([2H])c([2H])c(-c2c([2H])c([2H])c(N(c3c([2H])c([2H])c([2H])c4oc5c([2H])c(C(C)(C)C)c([2H])c([2H])c5c34)c3c([2H])c([2H])c([2H])c4oc5c([2H])c([2H])c(-c6c([2H])c([2H])c([2H])c([2H])c6[2H])c([2H])c5c34)c([2H])c2[2H])c([2H])c1[2H]]

### (1) Synthesis of P 1-106

Sub 1-106 (10.0 g, 20.8 mmol), Sub 2-106 (3.8 g, 20.8 mmol), Pd₂(dba)₃ (0.6 g, 0.6 mmol), t-BuONa (4.0 g, 41.5 mmol) were placed in a round-bottom flask and dissolved in anhydrous Toluene (104 ml, then P(t-Bu)₃ (50 wt % Sol.) (0.5 mL, 1.3 mmol) was added and heated and stirred at 110° C. for about 4 hours. After confirming the completion of the reaction by TLC, cooled to room temperature and extracted with CH₂Cl₂ and water. The separated organic layer was dried over MgSO₄, concentrated, and the resulting compound was recrystallized using silicagel column (Hexane CH₂Cl₂=4:1) to obtain 10.1 g (yield: 77%) of P 1-106.

### (2) Synthesis of P-106

P 1-106 (10.1 g, 16 mmol) obtained in the above synthesis was dissolved in perdeuterated benzene (C₆D₆) (120.7 g, 1,434.2 mmol) and CF₃SO₃D (12.0 g, 79.7 mmol) was added thereto, followed by reacting a temperature at 8000.

Samples were taken periodically for 3 hours and the degree of deuteration was measured by LC-MS, after the deuterium exchange reaction is completed at the desired substitution rate, cool to room temperature, quench by adding Na₂CO₃ in D₂O, and the organic solvent is concentrated. Recrystallization using toluene and acetone solvent gave 9.8 g (yield: 94%) of deuterated compound P-106. The final mass was determined by LC-MS to confirm to be 74.3% deuterated.

### 3. Synthesis Example of P-119

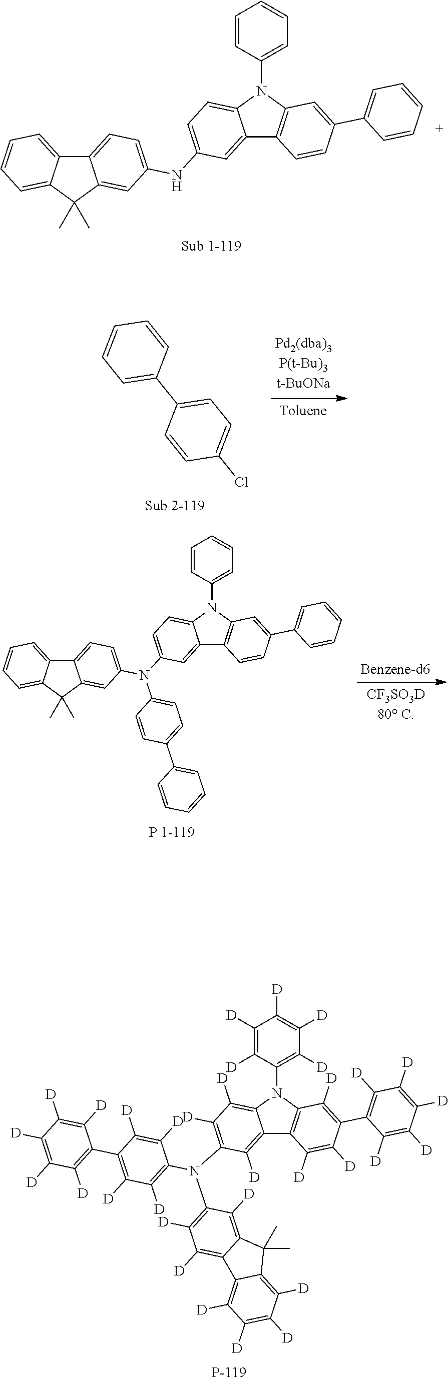 [SMILES: CC1(C)c2ccccc2-c2ccc(Nc3ccc4c(c3)c3ccc(-c5ccccc5)cc3n4-c3ccccc3)cc21 ; Clc1ccc(-c2ccccc2)cc1 ; CC1(C)c2ccccc2-c2ccc(N(c3ccc(-c4ccccc4)cc3)c3ccc4c(c3)c3ccc(-c5ccccc5)cc3n4-c3ccccc3)cc21 ; [2H]c1c([2H])c([2H])c(-c2c([2H])c([2H])c(N(c3c([2H])c([2H])c4c(c3[2H])C(C)(C)c3c([2H])c([2H])c([2H])c([2H])c3-4)c3c([2H])c([2H])c4c(c3[2H])c3c([2H])c([2H])c(-c5c([2H])c([2H])c([2H])c([2H])c5[2H])c([2H])c3n4-c3c([2H])c([2H])c([2H])c([2H])c3[2H])c([2H])c2[2H])c([2H])c1[2H]]

### (1) Synthesis of P 1-119

Sub 1-119 (10.0 g, 19.0 mmol), Sub 2-119 (3.5 g, 19.0 mmol), Pd₂(dba)₃ (0.5 g, 0.6 mmol), t-BuONa (3.6 g, 38.0 mmol) were placed in a round-bottom flask and dissolved in anhydrous Toluene (95 mL), then P(t-Bu)₃ (50 wt % Sol.) (0.5 mL, 1.1 mmol) was added and heated and stirred at 110° C. for about 4 hours. After confirming the completion of the reaction by TLC, cooled to room temperature and extracted with CH₂Cl₂ and water. The separated organic layer was dried over MgSO₄, concentrated, and the resulting compound was recrystallized using silicagel column (Hexane:CH₂Cl₂=4:1) to obtain 10.1 g (yield: 78%) of P 1-119.

### (2) Synthesis of P-119

P 1-119 (10.1 g, 14.8 mmol) obtained in the above synthesis was dissolved in perdeuterated benzene (C₆D₆) (112.7 g, 1,339.0 mmol) and CF₃SO₃D (11.2 g, 74.4 mmol) was added thereto, followed by reacting a temperature at 80° C.

Samples were taken periodically for 3 hours and the degree of deuteration was measured by LC-MS, after the deuterium exchange reaction is completed at the desired substitution rate, cool to room temperature, quench by adding Na₂CO₃ in D₂O, and the organic solvent is concentrated. Recrystallization using toluene and acetone solvent gave 9.8 g (yield: 93%) of deuterated compound P-119. The final mass was determined by LC-MS to confirm to be 84.2% deuterated.

### 4. Synthesis Example of P-124

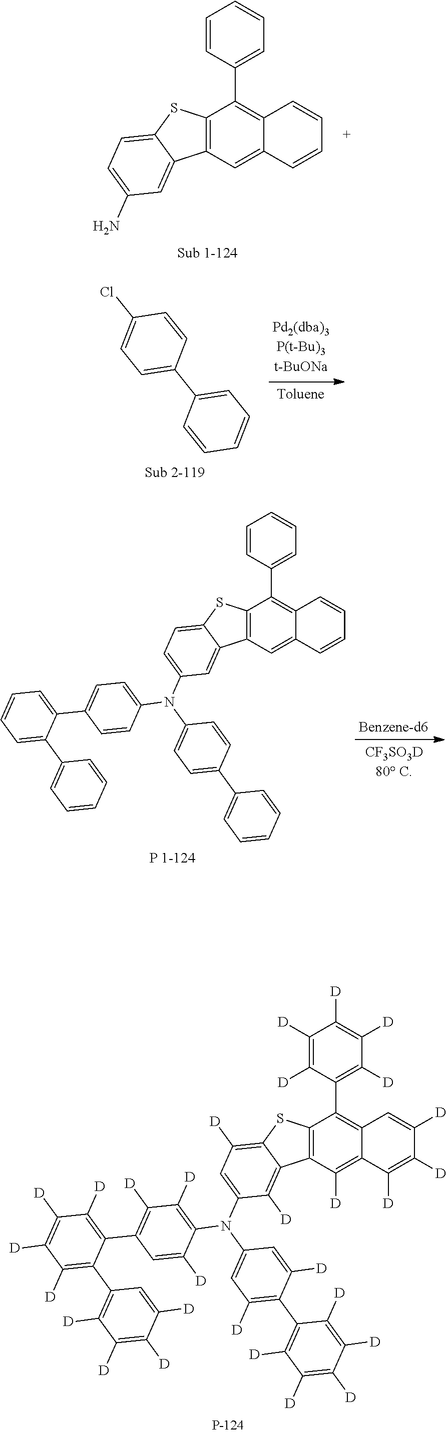 [SMILES: Nc1ccc2sc3c(-c4ccccc4)c4ccccc4cc3c2c1 ; Clc1ccc(-c2ccccc2)cc1 ; c1ccc(-c2ccc(N(c3ccc(-c4ccccc4-c4ccccc4)cc3)c3ccc4sc5c(-c6ccccc6)c6ccccc6cc5c4c3)cc2)cc1 ; [2H]c1cc(-c2c([2H])c([2H])c([2H])c([2H])c2-c2cc([2H])c(N(c3cc([2H])c(-c4c([2H])c([2H])c([2H])c([2H])c4[2H])c([2H])c3)c3cc([2H])c4sc5c(-c6c([2H])c([2H])c([2H])c([2H])c6[2H])c6cc([2H])c([2H])c([2H])c6c([2H])c5c4c3[2H])c([2H])c2[2H])c([2H])c([2H])c1[2H]]

### (1) Synthesis of P 1-124

Sub 1-124 (10.0 g, 30.7 mmol), Sub 2-119 (5.8 g, 30.7 mmol), Pd₂(dba)₃ (0.8 g, 0.9 mmol), t-BuONa (5.9 g, 1.8 mmol) were placed in a round-bottom flask and dissolved in anhydrous Toluene (154 mL), then P(t-Bu)₃ (50 wt % Sol.) (2.1 mL, 1.8 mmol) was added and heated and stirred at 110° C. for about 4 hours. After confirming the completion of the reaction by TLC, cooled to room temperature and extracted with CH₂Cl₂ and water. The separated organic layer was dried over MgSO₄, concentrated, and the resulting compound was recrystallized using silicagel column (Hexane:CH₂Cl₂=4:1) to obtain 15.7 g (yield: 72.4%) of P 1-124.

### (2) Synthesis of P-124

P 1-124 (15.7 g, 22.2 mmol) obtained in the above synthesis was dissolved in perdeuterated benzene (C₆D₆) (168.4 g, 2,001.6 mmol) and CF₃SO₃D (16.7 g, 111.2 mmol) was added thereto, followed by reacting a temperature at 80° C.

Samples were taken periodically for 3 hours and the degree of deuteration was measured by LC-MS, after the deuterium exchange reaction is completed at the desired substitution rate, cool to room temperature, quench by adding Na₂CO₃ in D₂O, and the organic solvent is concentrated. Recrystallization using toluene and acetone solvent gave 15.2 g (yield: 93%) of deuterated compound P-124. The final mass was determined by LC-MS to confirm to be 82.8% deuterated.

### Comparative Synthesis Example 1

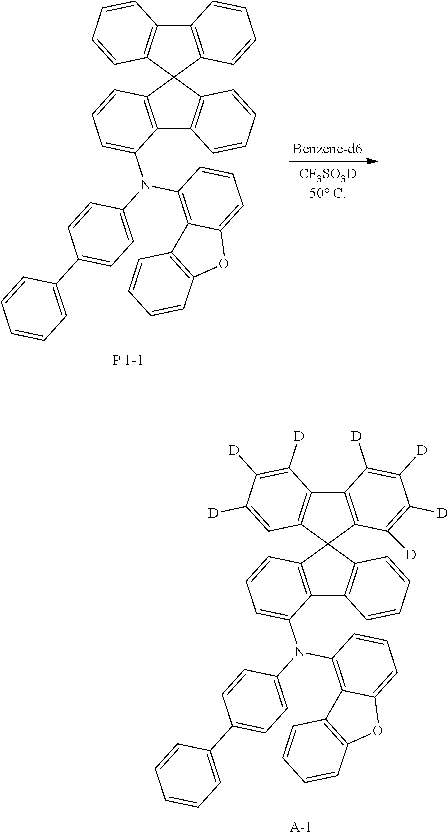

[Synthesis of Comparative Compound A-1]

P 1-1 (10.0 g, 15.4 mmol) obtained in the above synthesis was dissolved in perdeuterated benzene (C₆D₆) (116.5 g, 1,385.1 mmol) and CF₃SO₃D (11.6 g, 77.0 mmol) was added thereto, followed by reacting at a temperature of 50° C. for 20 hours to form a deuterated material. After the reaction is completed, cool to room temperature, quench by adding Na₂CO₃ in D₂O, and the organic solvent is concentrated. Recrystallization using toluene and acetone solvent gave 9.2 g (yield: 91%) of deuterated compound A-1. The final mass was determined by LC-MS to confirm to be 22.6% deuterated.

### Comparative Synthesis Example 2

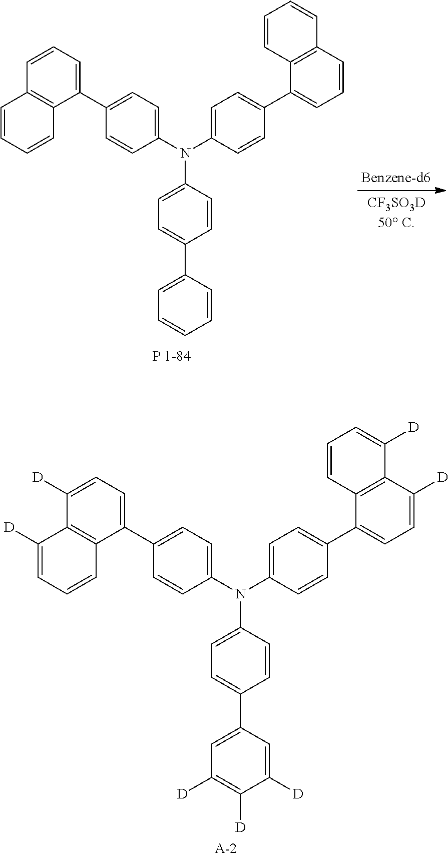

[Synthesis of Comparative Compound A-2]

P 1-84 (9.0 g, 15.7 mmol) obtained in the above synthesis was dissolved in perdeuterated benzene (C₆D₆) (118.8 g, 1,411.8 mmol) and CF₃SO₃D (11.8 g, 78.4 mmol) was added thereto, followed by reacting at a temperature of 50° C. for 20 hours to form a deuterated material. After the reaction is completed, cool to room temperature, quench by adding Na₂CO₃ in D₂O, and the organic solvent is concentrated. Recrystallization using toluene and acetone solvent gave 8.5 g (yield: 94%) of deuterated compound A-2. The final mass was determined by LC-MS to confirm to be 22.6% deuterated.

### Comparative Synthesis Example 3

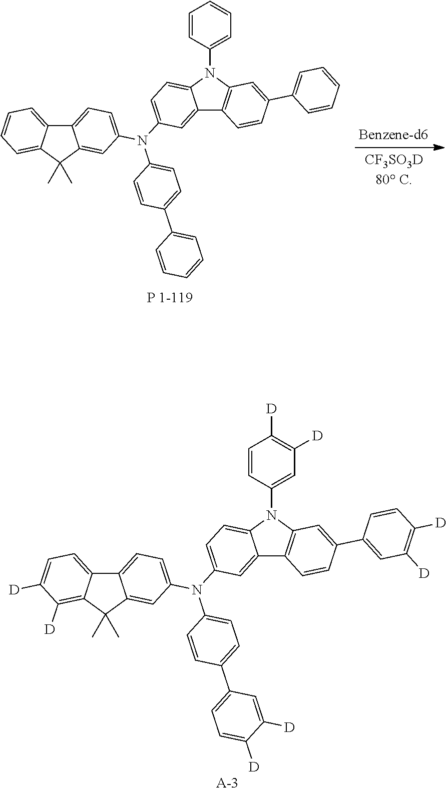

[Synthesis of Comparative Compound A-3]

P 1-119 (10.0 g, 14.7 mmol) obtained in the above synthesis was dissolved in perdeuterated benzene (C₆D₆) (111.6 g, 1,325.7 mmol) and CF₃SO₃D (11.1 g, 73.7 mmol) was added thereto, followed by reacting at a temperature of 50° C. for 20 hours to form a deuterated material. After the reaction is completed, cool to room temperature, quench by adding Na₂CO₃ in D₂O, and the organic solvent is concentrated. Recrystallization using toluene and acetone solvent gave 9.4 g (yield: 93%) of deuterated compound A-3. The final mass was determined by LC-MS to confirm to be 21.1% deuterated.

As can be seen from the results of Comparative Synthesis Examples 1 to 3, it can be seen that the conventionally known general deuterium substitution method has a long reaction time and is difficult to control the substitution rate with a significantly lower deuterium substitution rate compared to the manufacturing method of the present invention. In the manufacturing method described in the present invention, it is possible to reduce the reaction time by reacting at a high temperature compared to the existing reaction temperature for a certain time, preferably 3 hours, and to obtain a deuterated compound having an improved substitution rate.

Meanwhile, FD-MS (Field Desorption-Mass Spectrometry) values of the compounds of the present invention prepared according to the above synthesis examples are shown in Table 1.

TABLE 1

| compound | FD-MS | compound | FD-MS |
|---|---|---|---|
| P-1 | m/z = 676.41(C₄₉H₄D₂₇NO = 676.96) | P-2 | m/z = 692.40(C₄₉H₄D₂₇NO₂ = 692.96) |
| P-3 | m/z = 680.44(C₄₉H₄D₂₉NO = 680.99) | P-4 | m/z = 718.49(C₅₃H₆D₃₁N = 719.08) |
| P-5 | m/z = 755.49(C₅₅H₉D₂₉N₂ = 756.10) | P-6 | m/z = 765.44(C₅₅H₇D₂₇N₂O = 766.05) |
| P-7 | m/z = 719.49(C₅₃HD₃₄N = 720.08) | P-8 | m/z = 746.51(C₅₅H₂D₃₅N = 747.12) |
| P-9 | m/z = 825.56(C₆₁H₃D₃₈N = 826.24) | P-10 | m/z = 833.50(C₆₁H₇D₃₂NO = 834.18) |
| P-11 | m/z = 863.51(C₆₂H₅D₃₄NO₂ = 864.21) | P-12 | m/z = 876.58(C₆₅H₄D₃₉N = 877.30) |
| P-13 | m/z = 800.56(C₅₉H₄D₃₇N = 801.21) | P-14 | m/z = 912.58(C₆₇H₈D₃₆N₂ = 913.32) |
| P-15 | m/z = 702.46(C₅₂H₁₀D₂₇N = 703.04) | P-16 | m/z = 706.49(C₅₂H₁₀D₂₉N = 707.07) |
| P-17 | m/z = 717.45(C₅₂H₁₁D₂₆NO = 718.03) | P-18 | m/z = 546.34(C₃₉H₁₀D₁₉NO = 546.78) |
| P-19 | m/z = 545.34(C₃₉H₁₁D₁₈NO = 545.78) | P-20 | m/z = 557.30(C₃₉H₁₁D₁₆NO₂ = 557.75) |
| P-83 | m/z = 548.39(C₄₀H₄D₂₅N = 548.83) | P-84 | m/z = 598.4(C₄₄H₆D₂₅N = 598.89) |
| P-85 | m/z = 675.44(C₅₀H₉D₂₆N = 676) | P-86 | m/z = 650.43(C₄₈H₆D₂₇N = 650.96) |
| P-87 | m/z = 729.48(C₅₄H₇D₃₀N = 730.08) | P-88 | m/z = 679.47(C₅₀H₅D₃₀N = 680.02) |
| P-89 | m/z = 730.49(C₅₄H₆D₃₁N = 731.09) | P-90 | m/z = 677.45(C₅₀H₇D₂₈N = 678.01) |
| P-91 | m/z = 754.49(C₅₆H₁₀D₂₉N = 755.11) | P-92 | m/z = 752.48(C₅₆H₁₂D₂₇N = 753.1) |
| P-93 | m/z = 651.44(C₄₈H₅D₂₈N = 651.97) | P-94 | m/z = 703.47(C₅₂H₅D₃₀N = 704.04) |
| P-95 | m/z = 702.46(C₅₂H₆D₂₉N = 703.04) | P-96 | m/z = 780.5(C₅₈H₈D₃₁N = 781.15) |
| P-97 | m/z = 834.54(C₆₂H₆D₃₅N = 835.23) | P-98 | m/z = 886.57(C₆₆H₆D₃₇N = 887.3) |
| P-99 | m/z = 508.33(C₃₆H₄D₂₁NO = 508.73) | P-100 | m/z = 511.34(C₃₆HD₂₄NO = 511.75) |
| P-101 | m/z = 768.47(C₅₆H₈D₂₉NO = 769.09) | P-102 | m/z = 550.37(C₃₉H₆D₂₃NO = 550.81) |
| P-103 | m/z = 716.44(C₅₂H₈D₂₇NO = 717.02) | P-104 | m/z = 627.29(C₄₂H₅D₂₀NO₂S = 627.85) |
| P-105 | m/z = 887.54(C₆₄H₉D₃₃N₂O = 888.25) | P-106 | m/z = 659.43(C₄₆H₉D₂₆NO₂ = 659.95) |
| P-107 | m/z = 604.36(C₄₂H₄D₂₅NS = 604.91) | P-108 | m/z = 763.47(C₅₄H₅D₃₂NS = 764.15) |
| P-109 | m/z = 735.44(C₅₂H₅D₃₀NS = 736.1) | P-110 | m/z = 709.34(C₄₈H₇D₂₄NS₂ = 710.05) |
| P-111 | m/z = 498.43(C₄₉H₆D₂₉NS = 698.43) | P-112 | m/z = 887.48(C₆₀HD₃₇N₂S₂ = 888.32) |
| P-113 | m/z = 757.42(C₅₄H₇D₂₈NS = 758.11) | P-114 | m/z = 569.28(C₃₇H₃D₂₂NS₂ = 569.87) |
| P-115 | m/z = 600.37(C₄₂H₄D₂₄N₂O = 600.85) | P-116 | m/z = 679.42(C₄₈H₅D₂₇N₂O = 679.96) |
| P-117 | m/z = 756.46(C₅₄H₈D₂₈N₂O = 757.07) | P-118 | m/z = 716.46(C₅₂H₈D₂₈N₂ = 717.04) |
| P-119 | m/z = 710.50(C₅₁H₆D₃₂N₂ = 711.07) | P-120 | m/z = 838.55(C₆₀H₆D₃₅N₃ = 839.22) |
| P-121 | m/z = 671.48(C₄₈HD₃₃N₂ = 672.02) | P-122 | m/z = 770.5(C₅₆H₆D₃₂N₂ = 771.13) |
| P-123 | m/z = 651.37(C₄₆H₅D₂₄NO₂ = 651.89) | P-124 | m/z = 734.43(C₅₂H₆D₂₉NS = 735.1) |
| P-125 | m/z = 639.43(C₄₆H₅D₂₇N₂ = 639.94) | P-126 | m/z = 651.39(C₄₆H₅D₂₅N₂O = 651.91) |
| P-127 | m/z = 727.45(C₅₂H₉D₂₆N₃ = 728.03) | P-128 | m/z = 657.31(C₄₄H₃D₂₄NS₂ = 657.97) |
| P-129 | m/z = 682.45(C₄₉H₂D₃₁NO = 683.00) | P-130 | m/z = 800.47(C₅₇H₄D₃₃NS = 801.19) |

Manufacturing Evaluation of Organic Electric Elements

### [Example 1] Blue Organic Light Emitting Device (Emitting Auxiliary Layer)

After vacuum deposition of 2-TNATA to a thickness of 60 nm on the ITO layer (anode) formed on the glass substrate to form a hole injection layer, NPB was vacuum deposited on the hole injection layer to a thickness of 60 nm to form a hole transport layer. Then, the compound P-1 of the present invention was vacuum-deposited to a thickness of 20 nm on the hole transport layer to form an emitting auxiliary layer, and then 9,10-di(naphthalen-2-yl)anthracene as a host and BD-052X (manufactured by Idemitsu Kosan) as a dopant were used in a weight ratio of 96:4 on the emitting auxiliary layer to form an emitting layer with a thickness of 30 nm. Subsequently, (1,1′-biphenyl-4-olato)bis(2-methyl-8-quinolinolato)aluminum (hereinafter, BAlq) was vacuum-deposited to a thickness of 10 nm to form a hole blocking layer, and then on the hole blocking layer, bis(10-hydroxybenzo[h]quinolinato)beryllium (hereinafter, BeBq₂) was vacuum-deposited to a thickness of 40 nm to form an electron transport layer. Thereafter, LiF, which is an alkali metal halide, was deposited to a thickness of 0.2 nm to form an electron injection layer, and then AI was deposited to a thickness of 150 nm to form a cathode, thereby manufacturing an organic electroluminescent device.

### [Example 2] to [Example 20] Blue Organic Electroluminescent Device (Emitting Auxiliary Layer)

An organic electroluminescent device was manufactured in the same manner as in Example 1, except that the compound of the present invention described in Table 2 was used instead of the compound P-1 of the present invention as a emitting auxiliary layer material.

### [Comparative Example 1] to [Comparative Example 6]

An organic electroluminescent device was manufactured in the same manner as in Example 1, except that Comparative Compounds 1 to 6 described in Table 2 were used instead of Compound P-1 of the present invention as an emitting auxiliary layer material

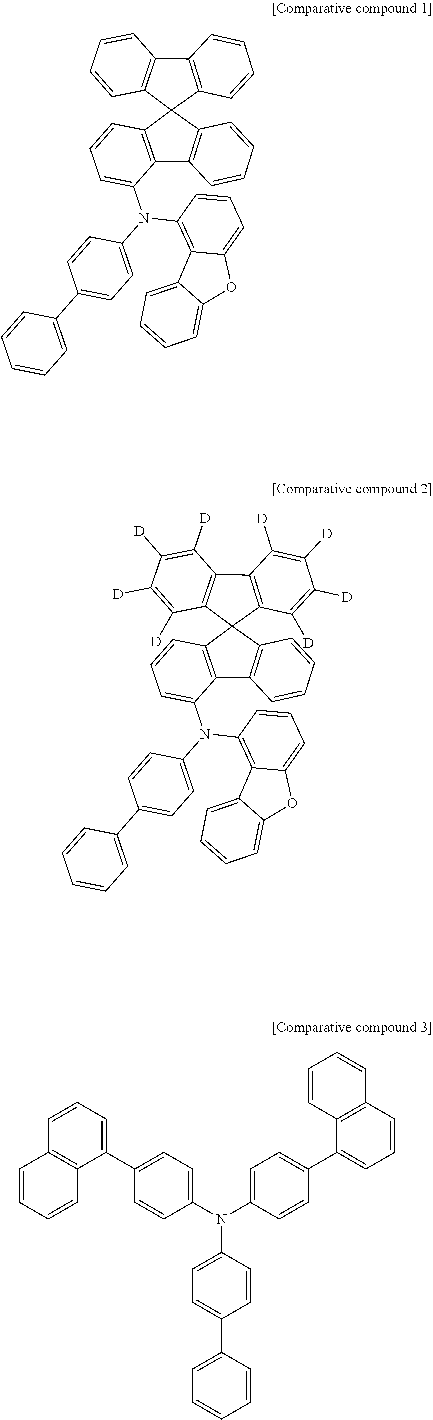 [SMILES: c1ccc(-c2ccc(N(c3cccc4c3-c3ccccc3C43c4ccccc4-c4ccccc43)c3cccc4oc5ccccc5c34)cc2)cc1 ; [2H]c1c([2H])c([2H])c2c(c1[2H])-c1c([2H])c([2H])c([2H])c([2H])c1C21c2ccccc2-c2c(N(c3ccc(-c4ccccc4)cc3)c3cccc4oc5ccccc5c34)cccc21 ; c1ccc(-c2ccc(N(c3ccc(-c4cccc5ccccc45)cc3)c3ccc(-c4cccc5ccccc45)cc3)cc2)cc1]

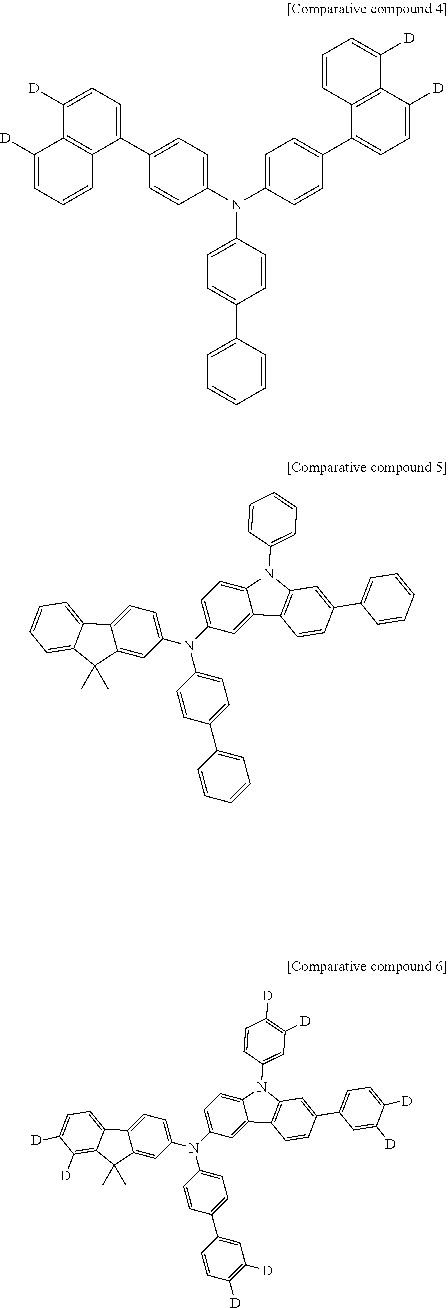 [SMILES: [2H]c1cccc2c(-c3ccc(N(c4ccc(-c5ccccc5)cc4)c4ccc(-c5ccc([2H])c6c([2H])cccc56)cc4)cc3)ccc([2H])c12 ; CC1(C)c2ccccc2-c2ccc(N(c3ccc(-c4ccccc4)cc3)c3ccc4c(c3)c3ccc(-c5ccccc5)cc3n4-c3ccccc3)cc21 ; [2H]c1ccc(-c2ccc(N(c3ccc4c(c3)C(C)(C)c3c-4ccc([2H])c3[2H])c3ccc4c(c3)c3ccc(-c5ccc([2H])c([2H])c5)cc3n4-c3ccc([2H])c([2H])c3)cc2)cc1[2H]]

By applying a forward bias DC voltage to the organic electroluminescent devices manufactured by Examples 1 to 20, Comparative Examples 1 to 6 of the present invention, Electroluminescence (EL) characteristics were measured with PR-650 from Photoresearch, and the T95 lifetime was measured using a lifetime measuring device manufactured by McScience at 500 cd/m² standard luminance, and the measurement results are shown in Table 2.

TABLE 2

| | | | Current | | | |
|---|---|---|---|---|---|---|
| | | | Density | Brightness | Efficiency | |
| | compound | Voltage | (mA/cm²) | (cd/m²) | (cd/A) | T(95) |
| comparative | comparative | 5.5 | 8.3 | 500.0 | 6.0 | 86.7 |
| example(1) | compound1 | | | | | |
| comparative | comparative | 5.3 | 7.7 | 500.0 | 6.5 | 97.9 |
| example(2) | compound2 | | | | | |
| comparative | comparative | 5.5 | 9.8 | 500.0 | 5.1 | 85.3 |
| example(3) | compound3 | | | | | |
| comparative | comparative | 5.4 | 9.3 | 500.0 | 5.4 | 95.7 |
| example(4) | compound4 | | | | | |
| comparative | comparative | 5.6 | 9.4 | 500.0 | 5.3 | 79.2 |
| example(5) | compound5 | | | | | |
| comparative | comparative | 5.5 | 8.9 | 500.0 | 5.6 | 89.1 |
| example(6) | compound6 | | | | | |
| example(1) | P-1 | 5.2 | 8.5 | 500.0 | 5.9 | 116.2 |
| example(2) | P-2 | 5.3 | 8.3 | 500.0 | 6.0 | 115.1 |
| example(3) | P-3 | 5.3 | 8.5 | 500.0 | 5.9 | 115.6 |
| example(4) | P-4 | 5.2 | 8.5 | 500.0 | 5.9 | 115.6 |
| example(5) | P-5 | 5.3 | 8.5 | 500.0 | 5.9 | 113.1 |
| example(6) | P-6 | 5.3 | 8.5 | 500.0 | 5.9 | 113.3 |
| example(7) | P-7 | 5.3 | 8.5 | 500.0 | 5.9 | 114.7 |
| example(8) | P-8 | 5.3 | 8.3 | 500.0 | 6.0 | 114.7 |
| example(9) | P-12 | 5.3 | 8.6 | 500.0 | 5.8 | 113.6 |
| example(10) | P-16 | 5.3 | 8.6 | 500.0 | 5.8 | 113.8 |
| example(11) | P-84 | 5.3 | 8.6 | 500.0 | 5.8 | 115.3 |
| example(12) | P-95 | 5.3 | 8.3 | 500.0 | 6.0 | 114.7 |
| example(13) | P-97 | 5.3 | 8.5 | 500.0 | 5.9 | 113.8 |
| example(14) | P-99 | 5.4 | 8.3 | 500.0 | 6.0 | 118.6 |
| example(15) | P-106 | 5.4 | 8.1 | 500.0 | 6.2 | 119.1 |
| example(16) | P-110 | 5.3 | 8.3 | 500.0 | 6.0 | 116.5 |
| example(17) | P-119 | 5.4 | 8.6 | 500.0 | 5.8 | 113.4 |
| example(18) | P-123 | 5.5 | 8.6 | 500.0 | 5.8 | 116.2 |
| example(19) | P-125 | 5.4 | 8.5 | 500.0 | 5.9 | 113.7 |
| example(20) | P-129 | 5.3 | 8.2 | 500.0 | 6.1 | 114.9 |

As can be seen from the results in Table 2, it can be seen that when a blue organic light emitting device is manufactured by using the material for an organic electroluminescent device of the present invention as an emitting auxiliary layer material, the lifespan of the organic electroluminescent device can be significantly improved compared to Comparative Examples using Comparative Compounds 1 to 6. In detail, compared to Comparative Compound 1, Comparative Compound 3, and Comparative Compound 5 in which were not substituted with deuterium, Comparative compound 2, comparative compound 4, and comparative compound 6 in which were partially substituted with deuterium showed improved device results with improved driving voltage, efficiency, and lifespan, and Compared to Comparative Compound 2, Comparative Compound 4, and Comparative Compound 6, the compound of the present invention in which 74% or more of the total hydrogen was substituted with deuterium showed superior device results in terms of lifespan.

When deuterium is substituted, as the bond length of deuterium-carbon becomes shorter than that of hydrogen-carbon, the molecular hardcore volume is reduced, and thus electrical polarizability can be reduced. For this reason, the effect of lowering the crystallinity of the thin film, that is, an amorphous state can be made, and consequently, hole mobility can be increased.

It can be seen that, in particular, the compound of the present invention deuterated at a substitution rate of 74% to 98%, which is higher than the existing substitution rate, increases the BDE (Bond Dissociation Energy) compared to the comparative compound, thereby maximizing the bond stability of the structure, and as a result, the stability of the molecules in the device is improved, so that the results are remarkably excellent in terms of lifespan.

This suggests that even though they have a similar structure, the physical properties, and properties of the compound, and results of the device may be significantly different depending on the substitution rate of deuterium.

In the case of the emitting auxiliary layer, it is necessary to understand the correlation between the hole transport layer and the emitting layer (host), and even if a similar core is used, it will be very difficult for a person skilled in the art to infer the characteristics exhibited by the emitting auxiliary layer in which the compound of the present invention is used.

In addition, in the evaluation results of the above-described device fabrication, the device characteristics in which the compound of the present invention is applied only to the emitting auxiliary layer has been described, but the compound of the present invention may be applied to the hole transport layer or both the hole transport layer and the emitting auxiliary layer may be applied.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

### INDUSTRIAL APPLICABILITY

According to the present invention, it is possible to manufacture an organic device having excellent device characteristics of high luminance, high light emission and long life, and thus has industrial applicability.

1. A 74% to 98% deuterated compound represented by Formula A: 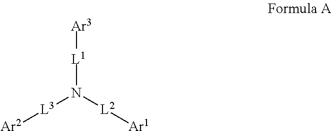
wherein:
1) L¹, L² and L³ are each independently selected from the group consisting of a single bond; a C₆-C₆₀ arylene group; a fluorenylene group; a fused ring group of a C₃-C₆₀ aliphatic ring and a C₆-C₆₀ aromatic ring; and a C₂-C₆₀ heterocyclic group,
2) Ar¹ and Ar² are each independently selected from the group consisting of a C₆-C₆₀ aryl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₆₀ alkyl group; a C₂-C₂₀ alkenyl group; a C₂-C₂₀ alkynyl group; a C₁-C₃₀ alkoxyl group; a C₆-C₆₀ aryloxy group; -L″-N(Ar^{a})(Ar^{b}); a substituent represented by Formula a; and a substituent represented by Formula b: 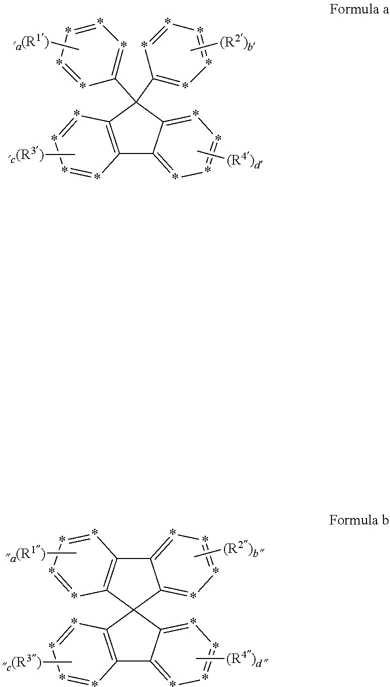
3) Ar³ is selected from the group consisting of a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₆₀ alkyl group; a C₂-C₂₀ alkenyl group; a C₂-C₂₀ alkynyl group; a C₁-C₃₀ alkoxyl group; a C₆-C₆₀ aryloxy group; and -L″-N(Ar^{a})(Ar^{b}),
4) R^{1′}, R^{2′}, R^{3′}, R^{4′}, R^{1″}, R^{2″}, R^{3″} and R^{4″} are each independently selected from the group consisting of hydrogen; deuterium; halogen; a cyano group; a nitro group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀alkenyl group; a C₂-C₂₀ alkynyl group; a C₁-C₃₀ alkoxyl group; a C₆-C₃₀ aryloxy group; and -L″-N(Ar^{a})(Ar^{b}); or in case a′, b′, c′, d′, a″, b, c“and d” are 2 or more, a′, b′, c′, d′, a″, b, c“and d” are each in plural being the same or different, and an adjacent plurality of R^{1′}s, plurality of R^{2′}s, plurality of R^{3′}s, plurality of R^{4′}s, plurality of R^{1″}s, plurality of R^{2″}s, plurality of R^{3″}s, or plurality of R^{4″}s may be bonded to each other to form a ring,
5) a′ and b′ are each independently an integer of 0 to 5, c′, d′, a″, b″, c″ and d″ are each independently an integer of 0 to 4,
6) any one of * is bonded to L² or L³ of Formula A,
7) wherein L″ is each independently selected from the group consisting of a single bond; a C₆-C₆₀ arylene group; a fluorenylene group; a fused ring group of a C₃-C₆₀ aliphatic ring and a C₆-C₆₀ aromatic ring; and a C₂-C₆₀ heterocyclic group including at least one heteroatom of O, N, S, Si or P; and wherein Ar^{a} and Ar^{b} are each independently selected from the group consisting of a C₆-C₆₀ aryl group; a fluorenyl group; a fused ring group of a C₃-C₆₀ aliphatic ring and a C₆-C₆₀ aromatic ring; and a C₂-C₆₀ heterocyclic group including at least one heteroatom of O, N, S, Si or P,
8) wherein the aryl group, arylene group, heterocyclic group, fluorenyl group, fluorenylene group, fused ring group, alkyl group, alkenyl group, alkynyl group, alkoxy group and aryloxy group may be substituted with one or more substituents selected from the group consisting of deuterium; halogen; a silane group; a siloxane group; a boron group; a germanium group; a cyano group; a nitro group; a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₂₀ aryl group; a C₆-C₂₀ aryl group substituted with deuterium; a fluorenyl group; a C₂-C₂₀ heterocyclic group; a C₃-C₂₀ cycloalkyl group; a C₇-C₂₀ arylalkyl group; a C₈-C₂₀ arylalkenyl group; or -L″-N(Ar^{a})(Ar^{b}), and the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

2. The compound of claim 1, wherein Ar³ is represented by Formula Ar-1 or Ar-2: 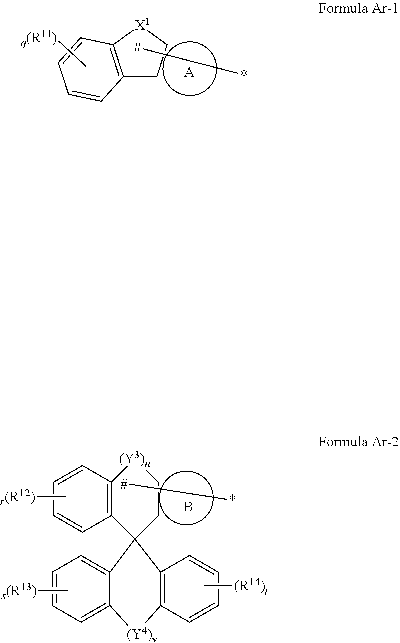
wherein:
1) * means a position bonded to L¹ of Formula A,
2) X¹ is O, S, NR^{a} or CR^{b}R^{c}, with the proviso that when # is bonded to X¹, X¹ is N,
3) Y³ and Y⁴ are each independently a single bond, O, S, NR^{d} or CR^{e}R^{f}, with the proviso that when # is bonded to Y³, Y³ is N,
4) R^{a}, R^{b}, R^{c}, R^{d}, R^{e} and R^{f} are each independently selected from the group consisting of a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; a C₆-C₆₀ aryl group; a fluorenyl group; a C₂-C₆₀ heterocyclic group including at least one heteroatom of O, N, S, Si or P; or R^{a}, R^{b}, R^{c}, R^{d}, R^{e} and R^{f} are bonded to adjacent groups to form a ring,
5) ring A and ring B are each independently selected from the group consisting of a C₆-C₆₀ aryl group; a C₂-C₆₀ heterocyclic group including at least one heteroatom of O, N, S, Si or P; and a fused ring group of a C₃-C₆₀ aliphatic ring and a C₆-C₆₀ aromatic ring; or ring A and ring B may be substituted with R²¹,
6) R¹¹, R¹², R¹³, R¹⁴ and R²¹ are each independently selected from the group consisting of hydrogen; deuterium; halogen; a cyano group; a C₆-C₆₀ aryl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxy group; a C₆-C₃₀ aryloxy group; and -L″-N(Ar^{a})(Ar^{b}); or R¹¹, R¹², R¹³, R¹⁴ and R²¹ are bonded to adjacent groups to form a ring,
7) wherein L″, Ar^{a} and Ar^{b} are the same as defined in claim 1,
8) q, r, s, and t are each independently an integer of 0 to 4, and
u and v are each independently 0 or 1 except that both u and v are 0, and where u or v is 0, Y³ or Y⁴ is absent.

3. The compound of claim 1, wherein Formula A is represented by Formula 1: 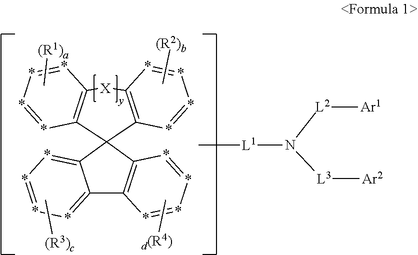
wherein:
1) X is a single bond,
2) y is an integer of 0 or 1,
3) i) when y is 0, a and b are each independently an integer of 0 to 5,
ii) when y is 1, a and b are each independently an integer of 0 to 4,
4) c and d are each independently an integer of 0 to 4,
5) R¹, R², R³ and R⁴ are the same as the definition of R¹ in claim 1,
6) L¹, L², L³, Ar¹ and Ar² are the same as defined in claim 1, and L¹ is bonded to any one of * in Formula 1.

4. The compound of claim 3, wherein Formula 1 is represented by any one of Formulas 2 to 4: 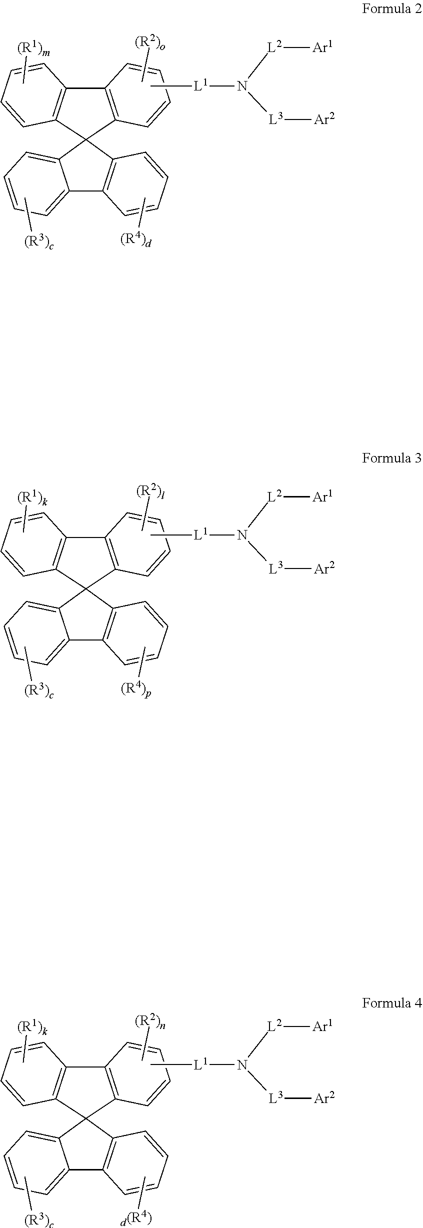
wherein:
1) L¹, L², L³, Ar¹ and Ar² are the same as defined in claim 3,
2) R¹, R², R³, R⁴, c and d are the same as defined in claim 3,
3) k and I are each independently an integer of 0 to 5, m and n are each independently an integer of 0 to 4, o and p are each independently an integer of 0 to 3.

5. The compound of claim 3, wherein Formula 1 is represented by Formula 5 or Formula 6: 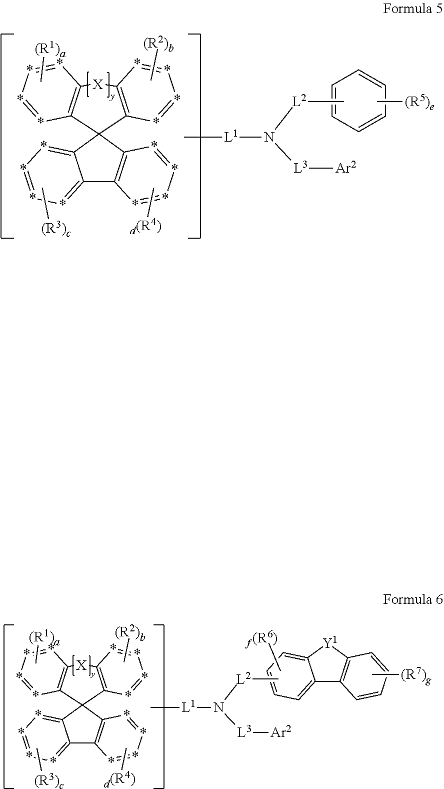
wherein:
1) L¹, L², L³ and Ar² are the same as defined in claim 3,
2) R¹, R², R³, R⁴, a, b, c, d, X and y are the same as defined in claim 3,
3) R⁵, R⁶ and R⁷ are the same as the definition of R¹ in claim 3,
4) e is an integer of 0 to 5, f is an integer of 0 to 3, g is an integer of 0 to 4,
5) Y¹ is O, S, CR′R″ or NR′,
6) wherein R′ and R″ are each independently selected from the group consisting of a C₆-C₆₀ aryl group; a fluorenyl group; a C₂-C₆₀ heterocyclic group including at least one heteroatom of O, N, S, Si or P; and a fused ring group of a C₃-C₆₀ aliphatic ring and a C₆-C₆₀ aromatic ring; and -L″-N(Ar^{a})(Ar^{b}); or R′ and R″ are bonded to each other to form a C₆-C₆₀ aromatic ring; a fluorenyl group; a C₂-C₆₀ heterocyclic group including at least one heteroatom of O, N, S, Si or P; a C₃-C₆₀ aliphatic ring; or a fused ring group of a C₃-C₆₀ aliphatic ring and a C₆-C₆₀ aromatic ring;
7) wherein L″ is each independently selected from the group consisting of a single bond: a C₆-C₆₀ arylene group: a fluorenylene group: a fused ring group of a C₃-C₆₀ aliphatic ring and a C₆-C₆₀ aromatic ring; and a C₂-C₆₀ heterocyclic group including at least one heteroatom of O, N, S, Si or P, and Ar^{a} and Ar^{b} are each independently selected from the group consisting of a C₆-C₆₀ aryl group: a fluorenyl group: a fused ring group of a C₃-C₆₀ aliphatic ring and a C₆-C₆₀ aromatic ring; and a C₂-C₆₀ heterocyclic group including at least one heteroatom of O, N, S, Si or P.

6. The compound of claim 3, wherein Formula 1 is represented by any one of Formulas 7 to 15: 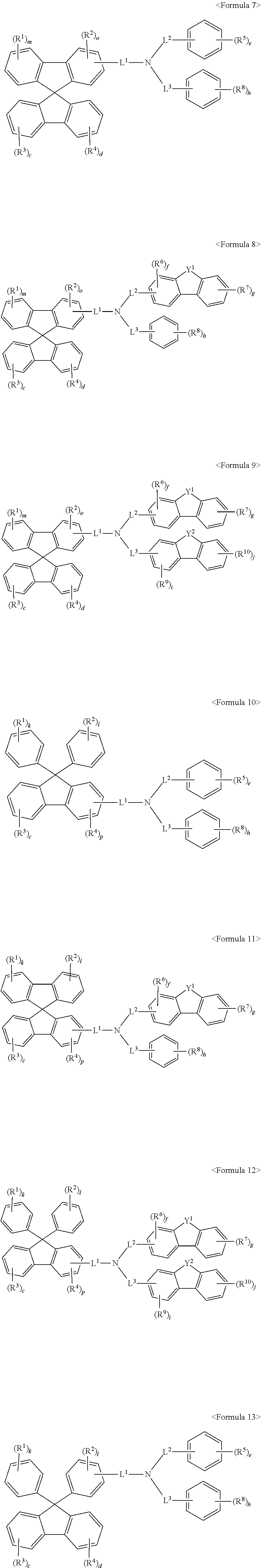 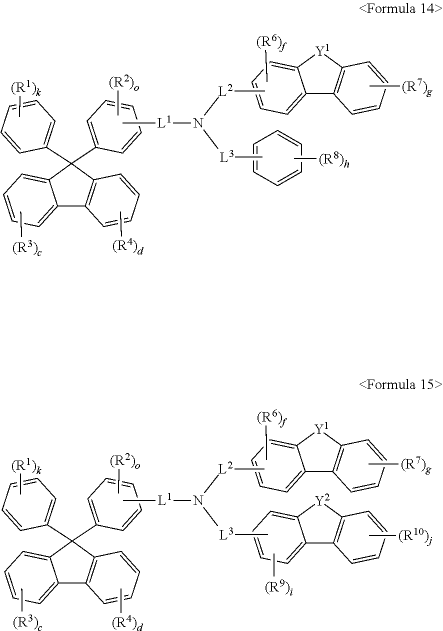
wherein:
1) L¹, L² and L³ are each independently selected from the group consisting of a single bond: a C₆-C₆₀ arylene group: a fluorenylene group: a fused ring group of a C₃-C₆₀ aliphatic ring and a C₃-C₆₀ aromatic ring; and a C₂-C₆₀ heterocyclic group,
2) R¹, R², R³, R⁴, R⁵, R⁶, R⁷, R⁸, R⁹ and R¹⁰ are each the same as the definition of R¹ in claim 3,
3) e, h, k and I are each independently an integer of 0 to 5, c, d, g, j, m and n are each independently an integer of 0 to 4, f, i, o and p are each independently an integer of 0 to 3,
4) Y¹ is O, S, CR′R″ or NR′,
5) Y² is O, S or CR^{x}R^{y},
6) wherein R^{x} and R^{y} are each independently selected from the group consisting of a C₃-C₅₀ alkyl group; a C₆-C₆₀ aryl group; a fluorenyl group; a C₃-C₆₀ heterocyclic group including at least one heteroatom of O, N, S, Si or P; or R^{x} and R^{y} are bonded to each other to form a C₆-C₆₀ aromatic ring; a fluorenyl group; a C₂-C₆₀ heterocyclic group including at least one heteroatom of O, N, S, Si or P; a C₃-C₆₀ aliphatic ring; or a fused ring group of a C₃-C₆₀ aliphatic ring and a C₆-C₆₀ aromatic ring.

7. The compound of claim 1, wherein Formula A is represented by any one of the following compounds P-1 to P-20 and P-83 to P-130:

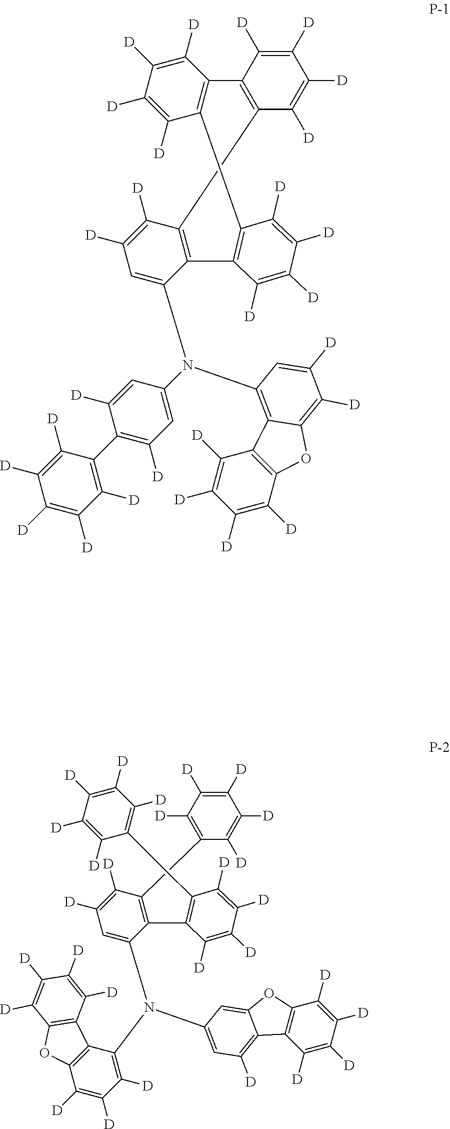 [SMILES: [2H]c1cc(N(c2cc([2H])c(-c3c([2H])c([2H])c([2H])c([2H])c3[2H])c([2H])c2)c2cc([2H])c([2H])c3oc4c([2H])c([2H])c([2H])c([2H])c4c23)c2c(c1[2H])-c1c([2H])c([2H])c([2H])c([2H])c1-c1c([2H])c([2H])c([2H])c([2H])c1-c1c([2H])c([2H])c([2H])c([2H])c1-2 ; [2H]c1cc(N(c2cc([2H])c3c(c2)oc2c([2H])c([2H])c([2H])c([2H])c23)c2c([2H])c([2H])c([2H])c3oc4c([2H])c([2H])c([2H])c([2H])c4c23)c(-c2c([2H])c([2H])c([2H])c([2H])c2-c2c([2H])c([2H])c([2H])c([2H])c2[2H])c(-c2c([2H])c([2H])c([2H])c([2H])c2[2H])c1[2H]]

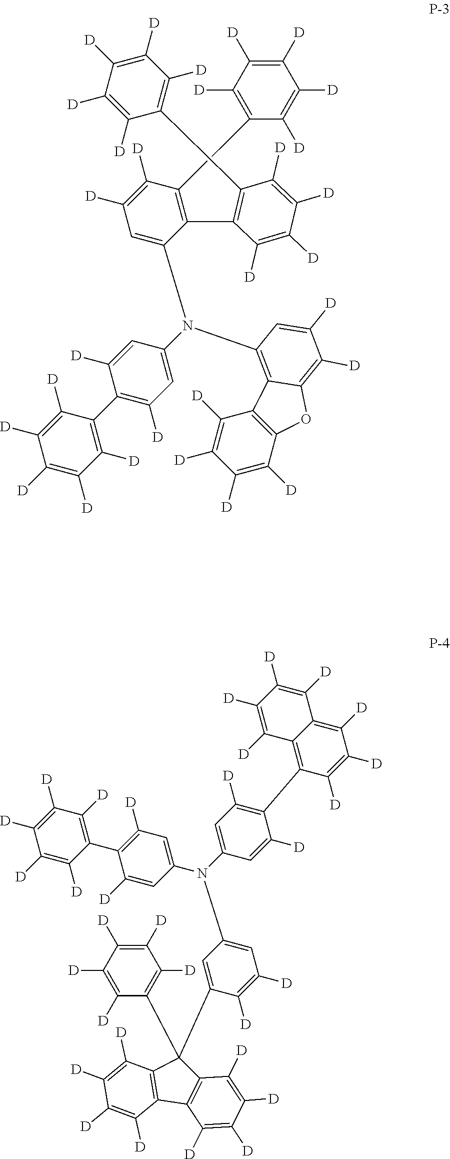

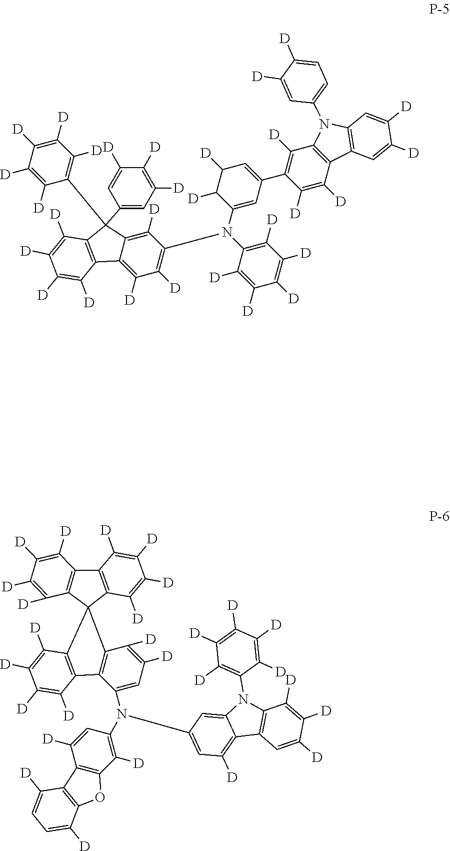

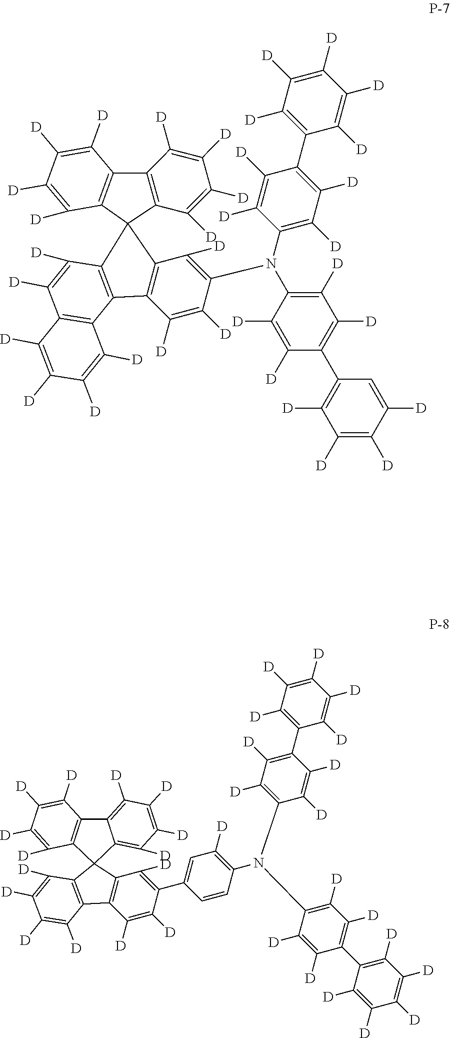

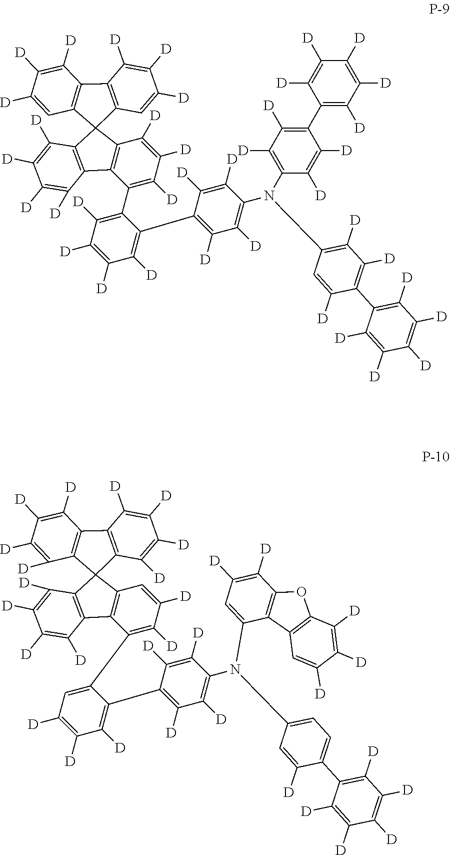

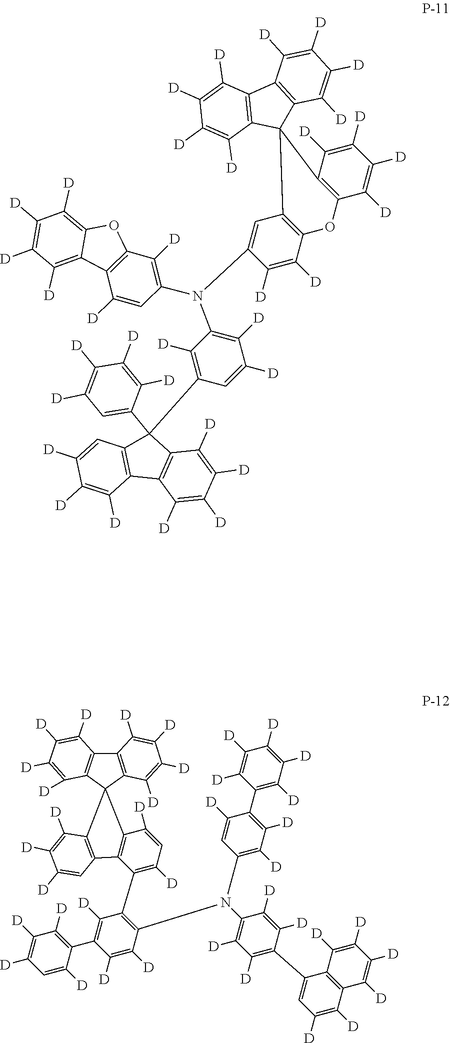

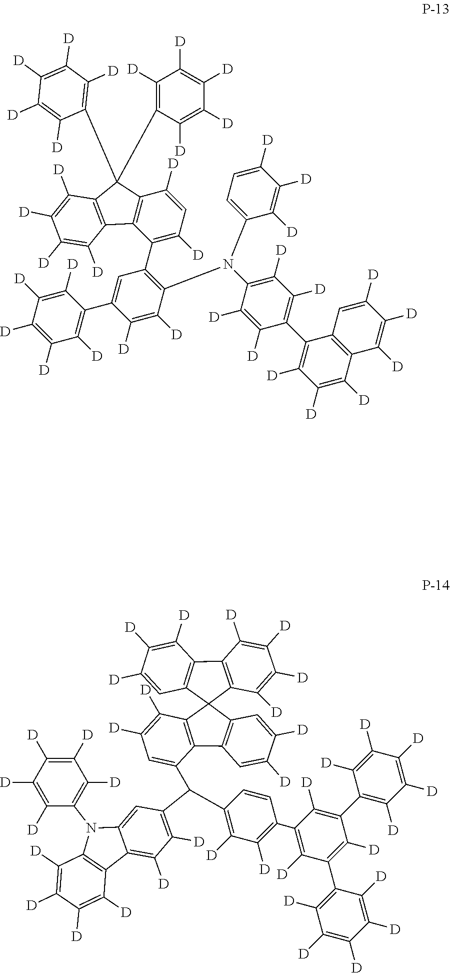

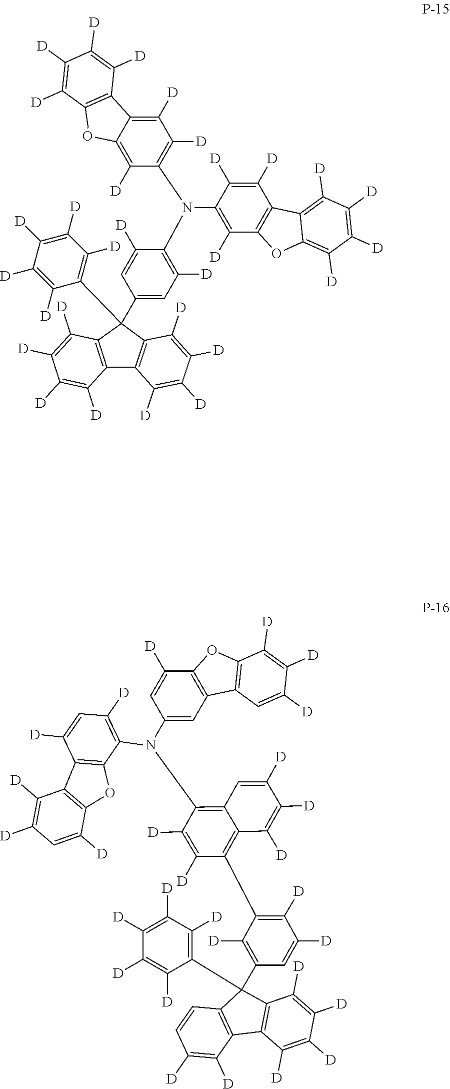

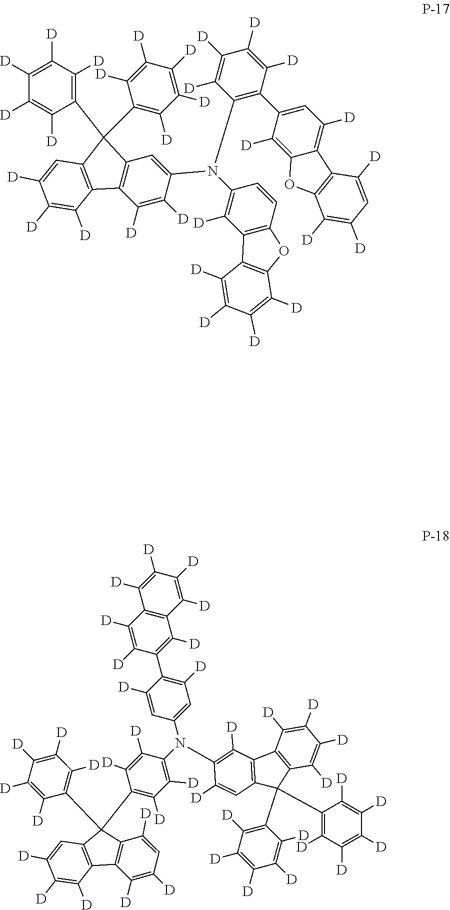

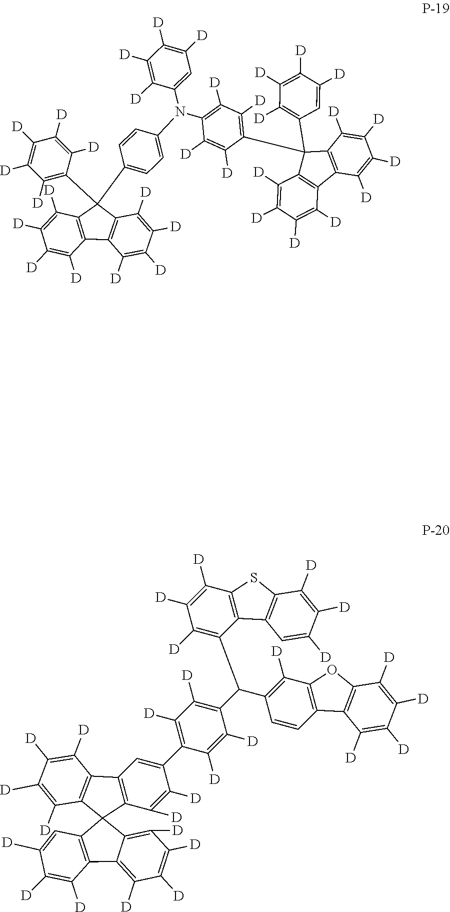

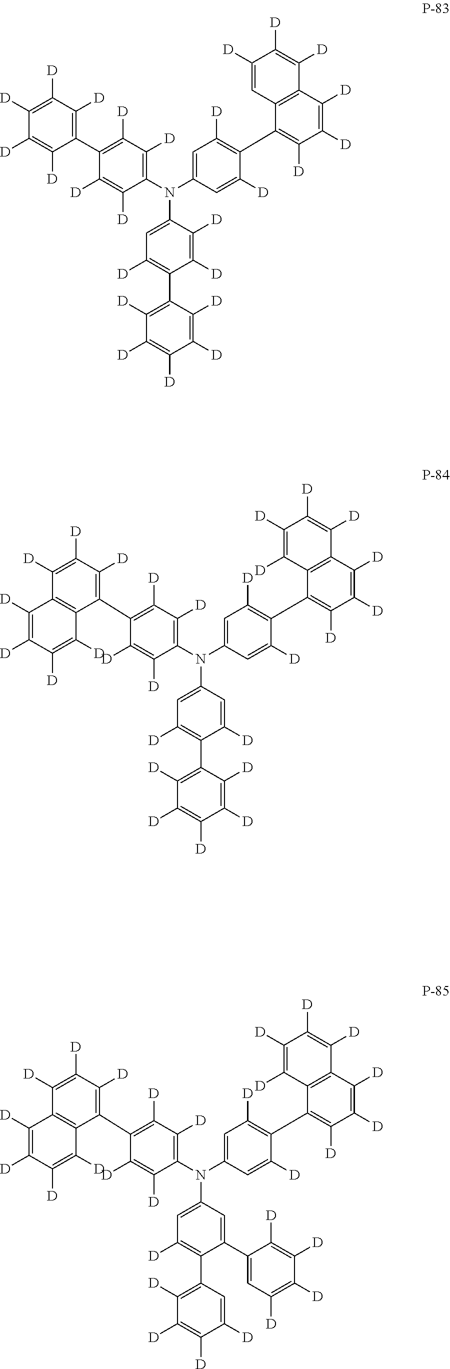

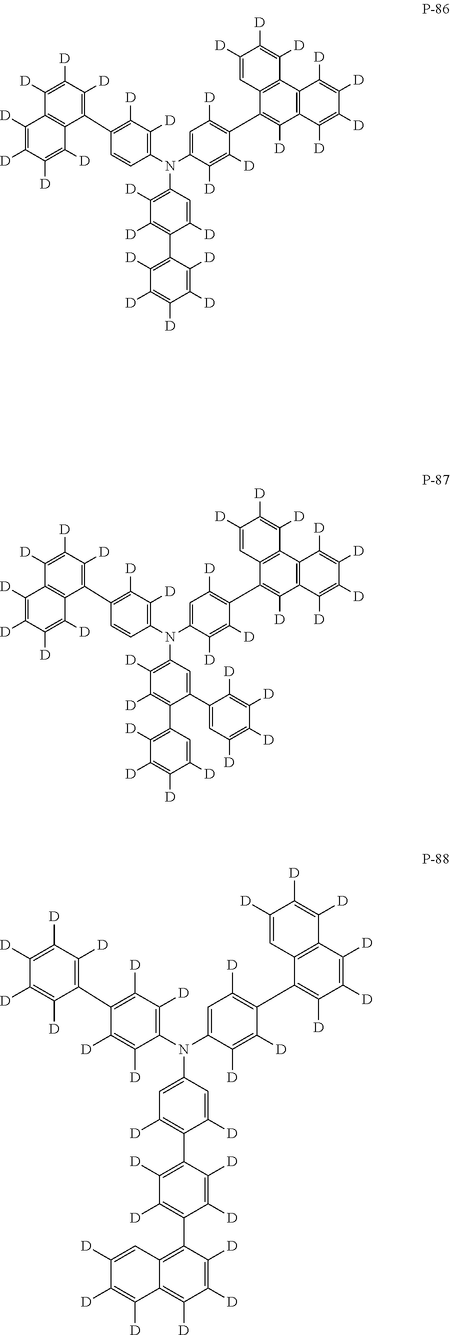

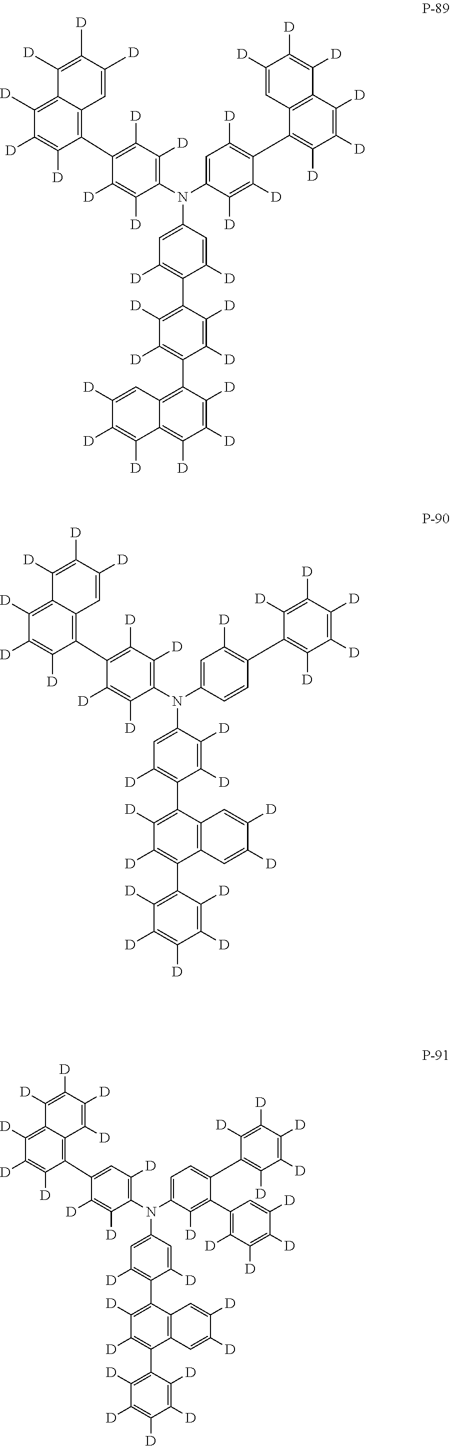

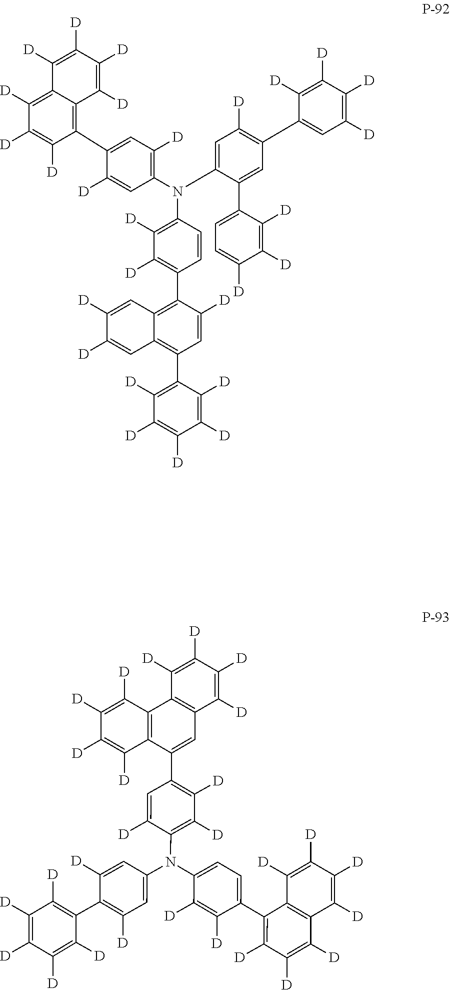 [SMILES: [2H]c1cc2c(-c3c([2H])c([2H])c([2H])c([2H])c3[2H])cc([2H])c(-c3ccc(N(c4cc([2H])c(-c5c([2H])c([2H])c([2H])c6c([2H])c([2H])c([2H])c([2H])c56)cc4[2H])c4cc([2H])c(-c5cc([2H])c([2H])c([2H])c5[2H])cc4-c4ccc([2H])c([2H])c4[2H])c([2H])c3[2H])c2cc1[2H] ; [2H]c1cc(N(c2ccc(-c3c([2H])c([2H])c([2H])c4c([2H])c([2H])c([2H])c([2H])c34)c([2H])c2[2H])c2c([2H])cc(-c3cc4c([2H])c([2H])c([2H])c([2H])c4c4c([2H])c([2H])c([2H])c([2H])c34)c([2H])c2[2H])cc([2H])c1-c1c([2H])c([2H])c([2H])c([2H])c1[2H]]

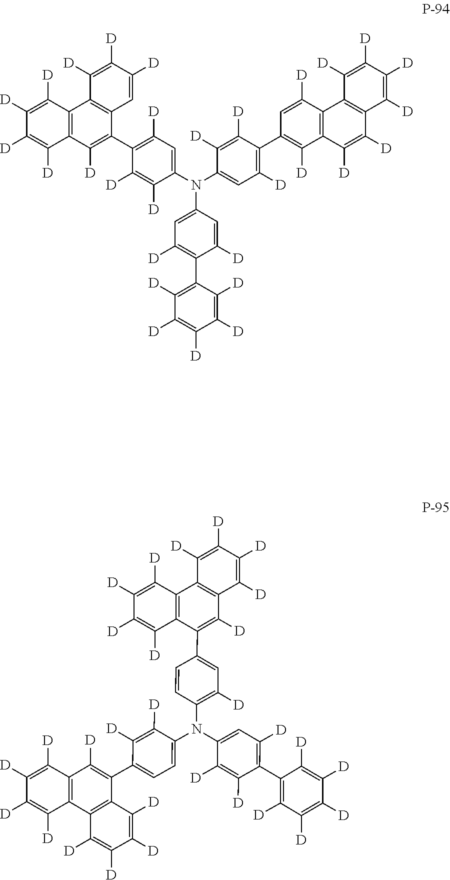

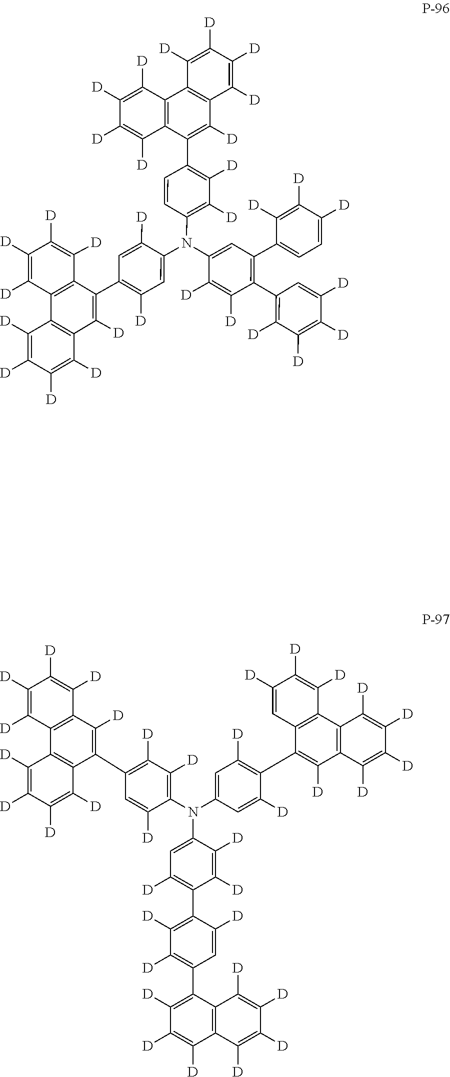

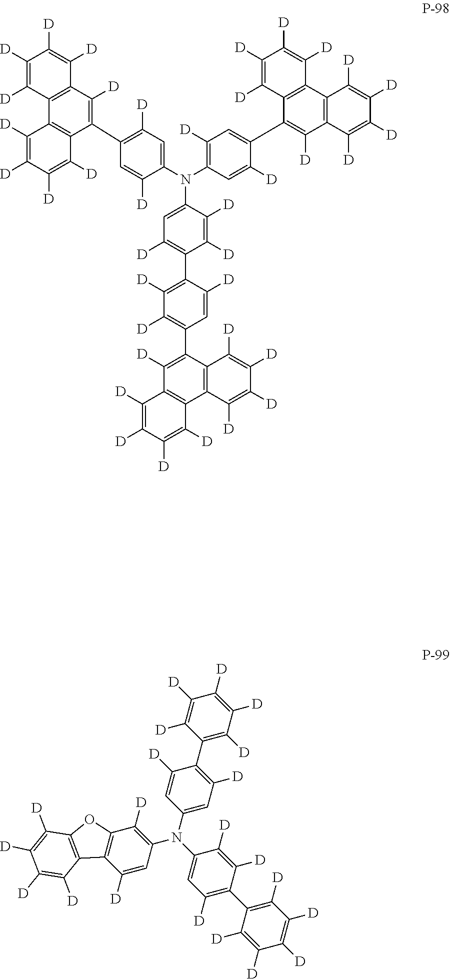

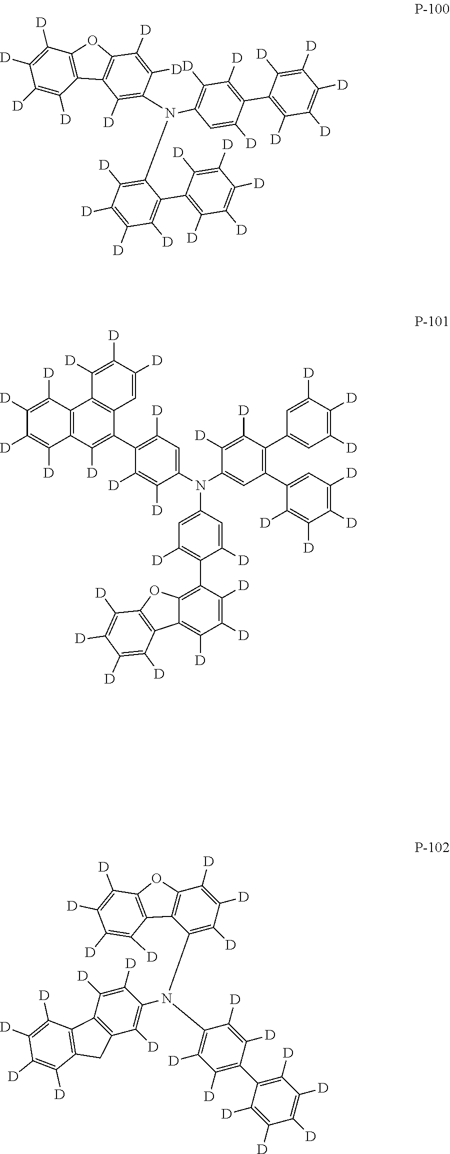

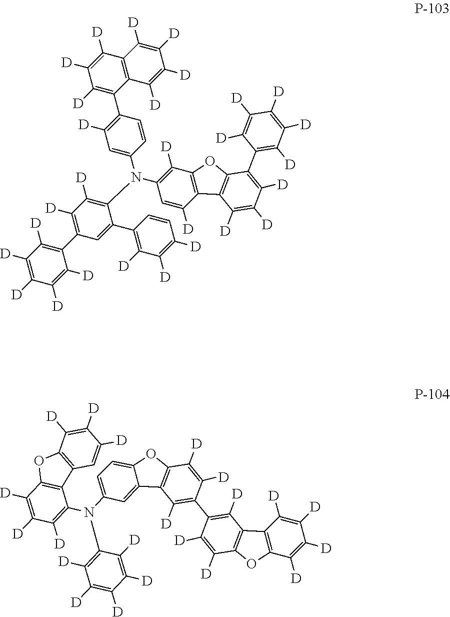 [SMILES: [2H]c1cc(N(c2cc([2H])c3c(oc4c(-c5c([2H])c([2H])c([2H])c([2H])c5[2H])c([2H])c([2H])c([2H])c43)c2[2H])c2c(-c3ccc([2H])c([2H])c3[2H])cc(-c3c([2H])c([2H])c([2H])c([2H])c3[2H])c([2H])c2[2H])ccc1-c1c([2H])c([2H])c([2H])c2c([2H])c([2H])c([2H])c([2H])c12 ; [2H]c1cc2c(oc3c([2H])c([2H])c([2H])c(N(c4ccc5oc6c([2H])c([2H])c(-c7c([2H])c([2H])c8oc9c([2H])c([2H])c([2H])c([2H])c9c8c7[2H])c([2H])c6c5c4)c4c([2H])c([2H])c([2H])c([2H])c4[2H])c32)c([2H])c1[2H]]

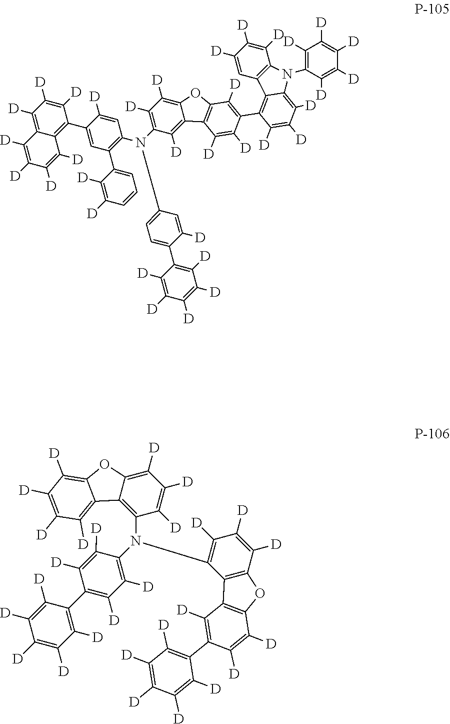

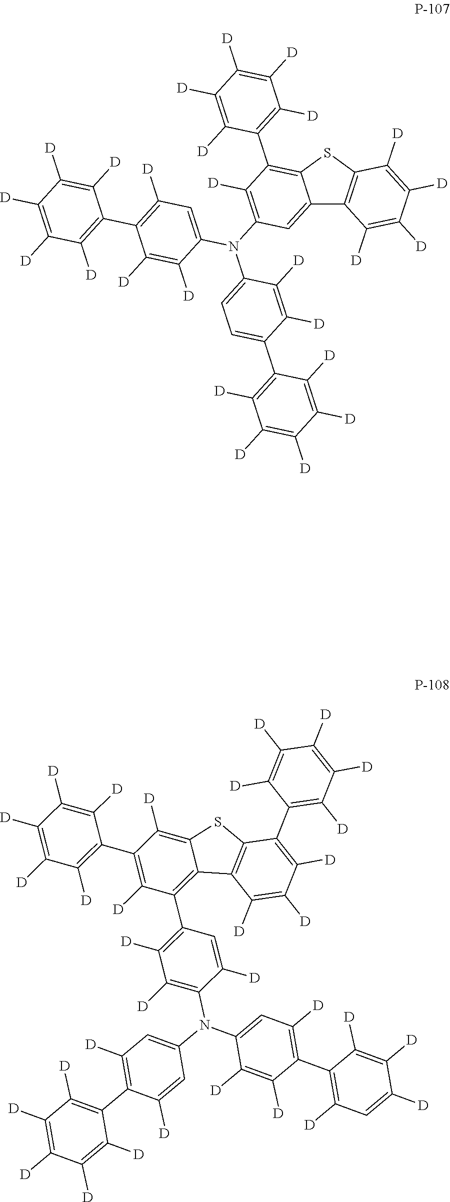 [SMILES: [2H]c1cc(N(c2ccc(-c3c([2H])c([2H])c([2H])c([2H])c3[2H])c([2H])c2[2H])c2cc3c(sc4c([2H])c([2H])c([2H])c([2H])c43)c(-c3c([2H])c([2H])c([2H])c([2H])c3[2H])c2[2H])c([2H])c([2H])c1-c1c([2H])c([2H])c([2H])c([2H])c1[2H] ; [2H]c1cc([2H])c(-c2c([2H])cc(N(c3cc([2H])c(-c4c([2H])c([2H])c([2H])c([2H])c4[2H])c([2H])c3)c3c([2H])cc(-c4c([2H])c(-c5c([2H])c([2H])c([2H])c([2H])c5[2H])c([2H])c5sc6c(-c7c([2H])c([2H])c([2H])c([2H])c7[2H])c([2H])c([2H])c([2H])c6c45)c([2H])c3[2H])c([2H])c2[2H])c([2H])c1[2H]]

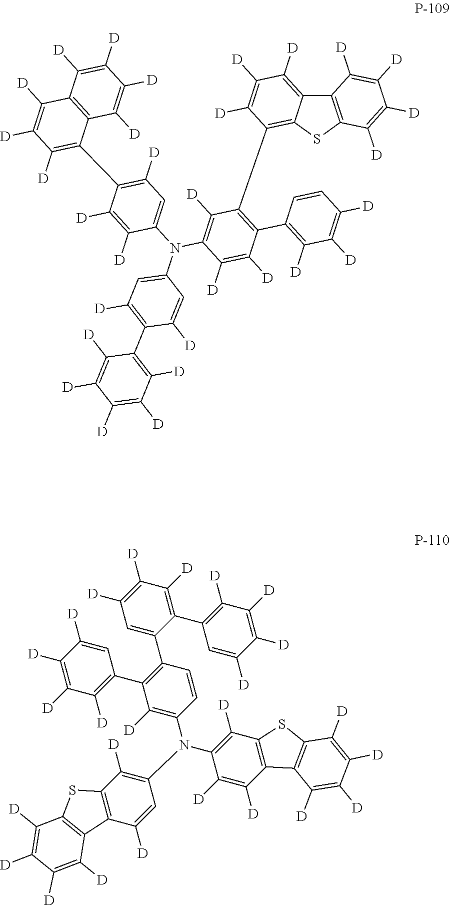 [SMILES: [2H]c1ccc(-c2c([2H])c([2H])c(N(c3cc([2H])c(-c4c([2H])c([2H])c([2H])c([2H])c4[2H])c([2H])c3)c3cc([2H])c(-c4c([2H])c([2H])c([2H])c5c([2H])c([2H])c([2H])c([2H])c45)c([2H])c3[2H])c([2H])c2-c2c([2H])c([2H])c([2H])c3c2sc2c([2H])c([2H])c([2H])c([2H])c23)c([2H])c1[2H] ; [2H]c1cc(-c2c(-c3cc([2H])c([2H])c([2H])c3-c3cc([2H])c([2H])c([2H])c3[2H])ccc(N(c3cc([2H])c4c(sc5c([2H])c([2H])c([2H])c([2H])c54)c3[2H])c3c([2H])c([2H])c4c(sc5c([2H])c([2H])c([2H])c([2H])c54)c3[2H])c2[2H])c([2H])c([2H])c1[2H]]

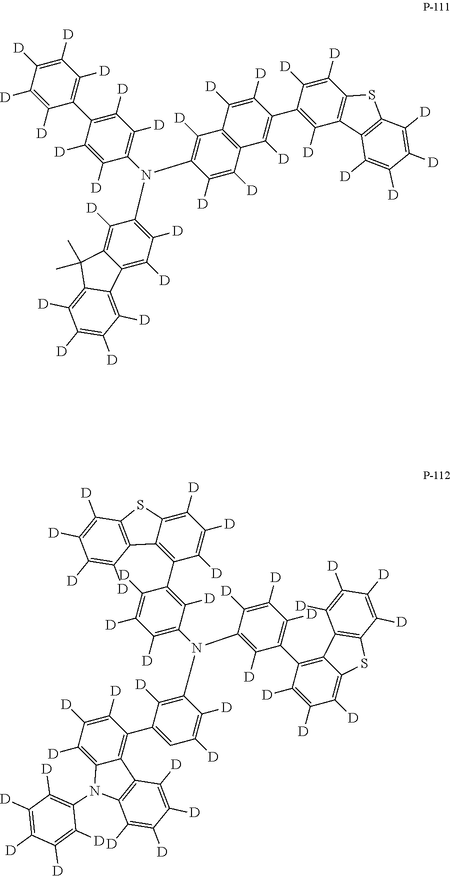

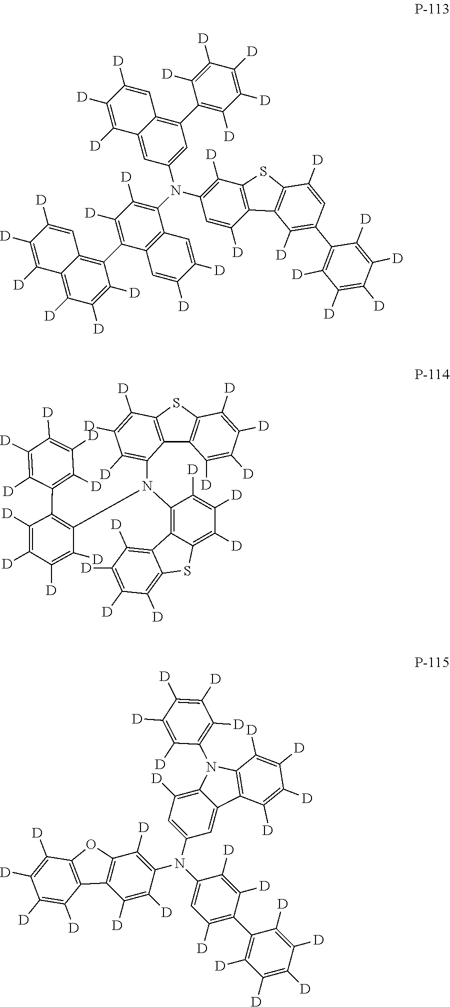

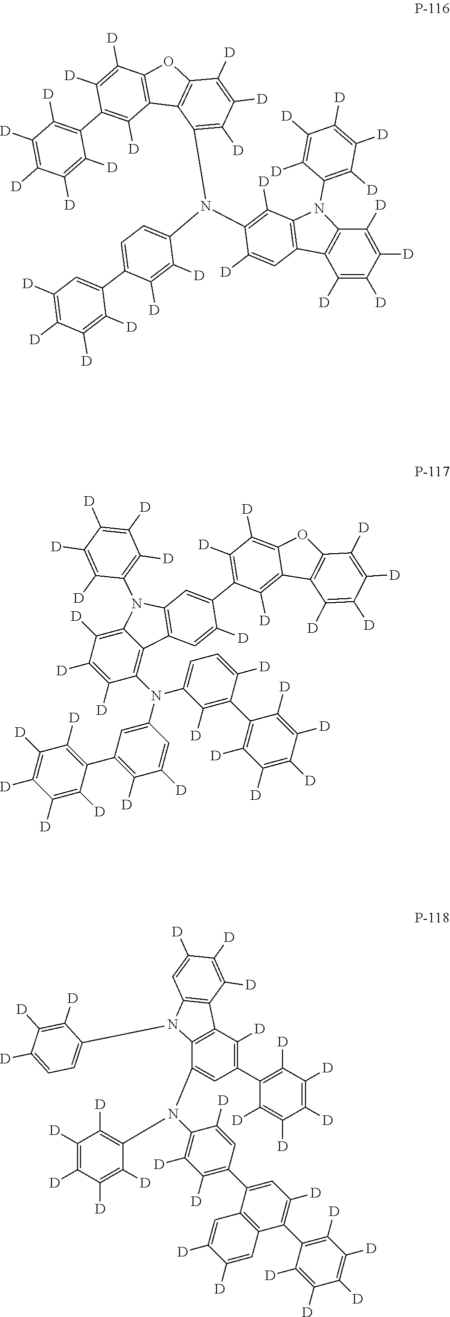

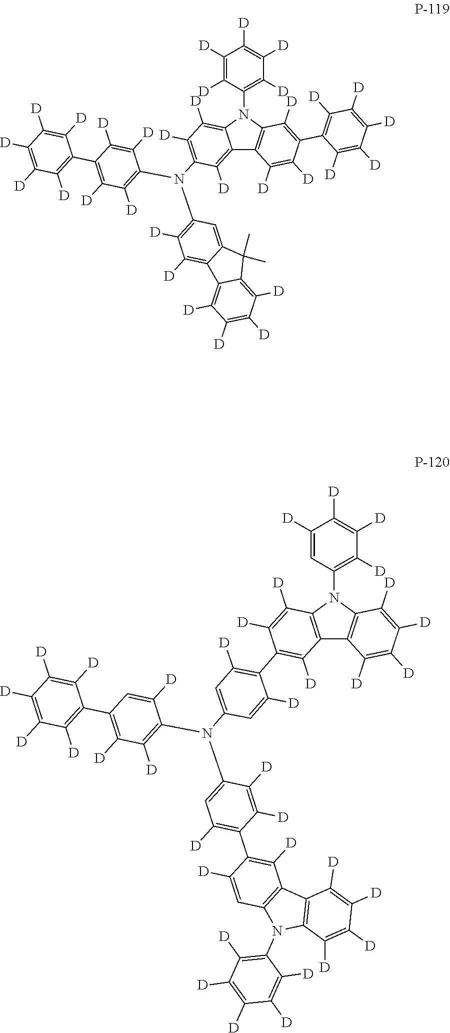

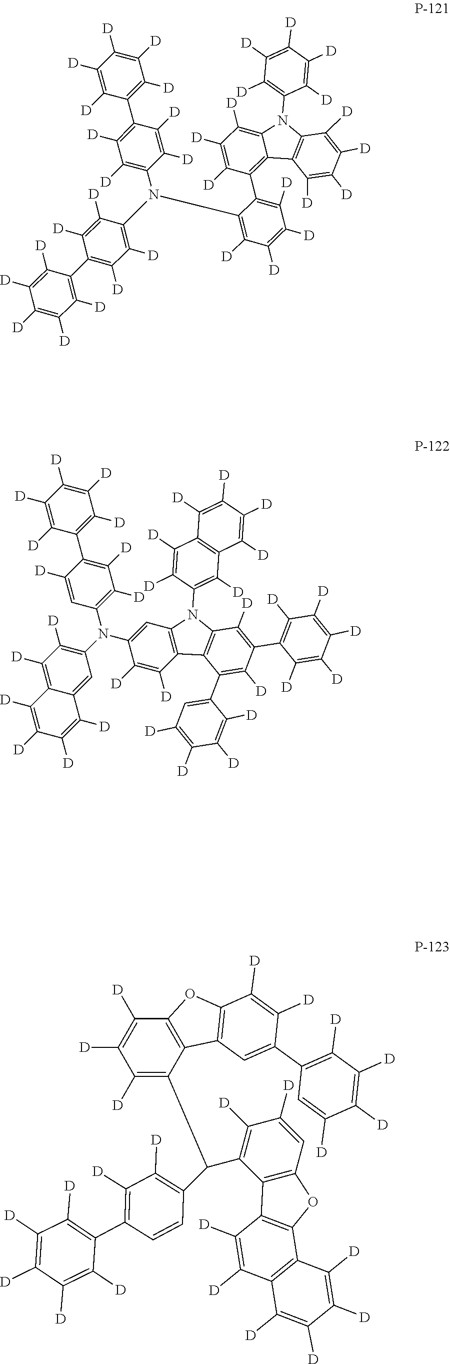

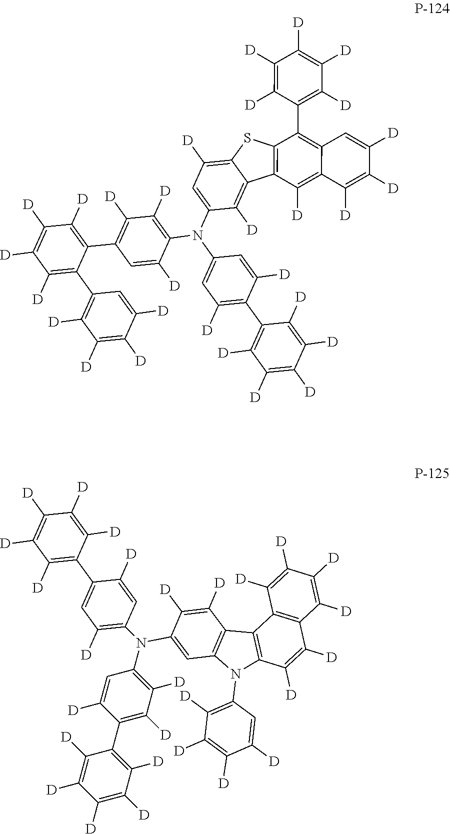 [SMILES: [2H]c1cc(-c2c([2H])c([2H])c([2H])c([2H])c2-c2cc([2H])c(N(c3cc([2H])c(-c4c([2H])c([2H])c([2H])c([2H])c4[2H])c([2H])c3)c3cc([2H])c4sc5c(-c6c([2H])c([2H])c([2H])c([2H])c6[2H])c6cc([2H])c([2H])c([2H])c6c([2H])c5c4c3[2H])c([2H])c2[2H])c([2H])c([2H])c1[2H] ; [2H]c1cc(N(c2cc([2H])c(-c3c([2H])c([2H])c([2H])c([2H])c3[2H])c([2H])c2[2H])c2cc3c(c([2H])c2[2H])c2c4c([2H])c([2H])c([2H])c([2H])c4c([2H])c([2H])c2n3-c2cc([2H])c([2H])c([2H])c2[2H])c([2H])cc1-c1c([2H])c([2H])c([2H])c([2H])c1[2H]]

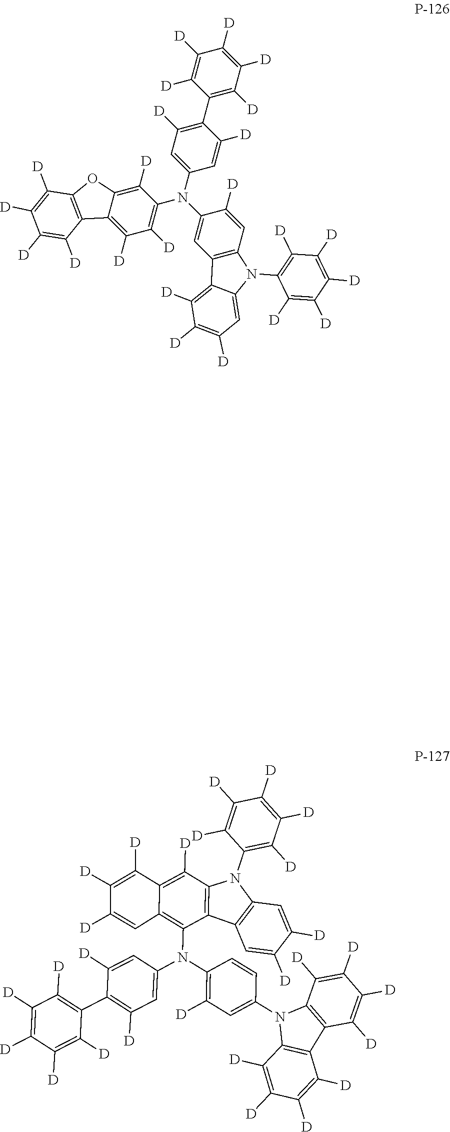 [SMILES: [2H]c1cc2c(cc1N(c1cc([2H])c(-c3c([2H])c([2H])c([2H])c([2H])c3[2H])c([2H])c1)c1c([2H])c([2H])c3c(oc4c([2H])c([2H])c([2H])c([2H])c43)c1[2H])c1c([2H])c([2H])c([2H])cc1n2-c1c([2H])c([2H])c([2H])c([2H])c1[2H] ; [2H]c1cc2c3c(N(c4cc([2H])c(-c5c([2H])c([2H])c([2H])c([2H])c5[2H])c([2H])c4)c4ccc(-n5c6c([2H])c([2H])c([2H])c([2H])c6c6c([2H])c([2H])c([2H])c([2H])c65)cc4[2H])c4cc([2H])c([2H])c([2H])c4c([2H])c3n(-c3c([2H])c([2H])c([2H])c([2H])c3[2H])c2cc1[2H]]

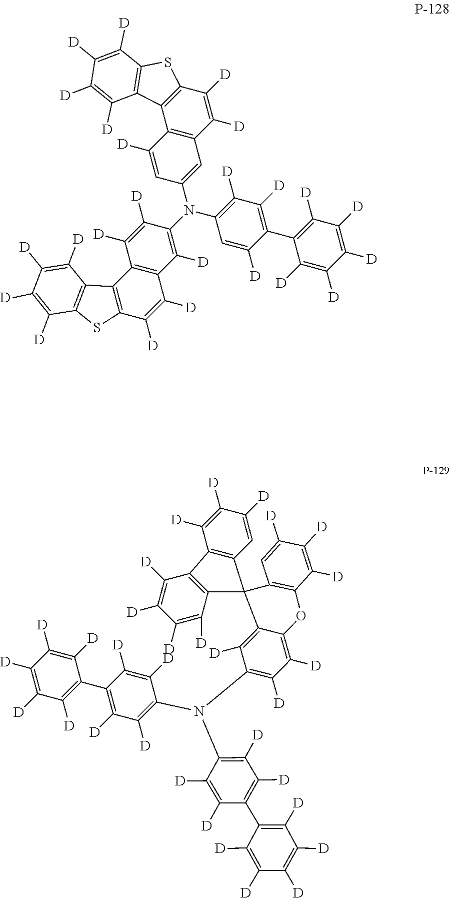 [SMILES: [2H]c1cc(N(c2cc([2H])c3c(c2)c([2H])c([2H])c2sc4c([2H])c([2H])c([2H])c([2H])c4c23)c2c([2H])c([2H])c3c(c2[2H])c([2H])c([2H])c2sc4c([2H])c([2H])c([2H])c([2H])c4c23)c([2H])c([2H])c1-c1c([2H])c([2H])c([2H])c([2H])c1[2H] ; [2H]c1cc2c(c([2H])c1[2H])Oc1c([2H])c([2H])c(N(c3c([2H])c([2H])c(-c4c([2H])c([2H])c([2H])c([2H])c4[2H])c([2H])c3[2H])c3c([2H])c([2H])c(-c4c([2H])c([2H])c([2H])c([2H])c4[2H])c([2H])c3[2H])c([2H])c1C21c2cc([2H])c([2H])c([2H])c2-c2c([2H])c([2H])c([2H])c([2H])c21]

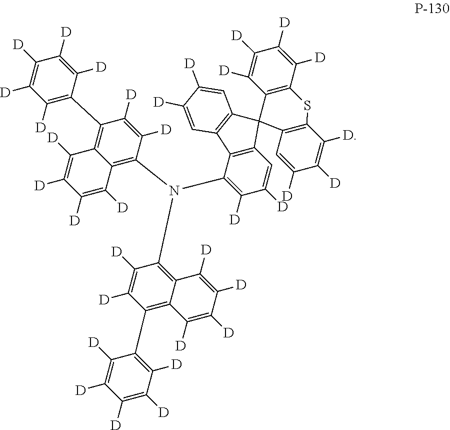

8. A method for preparing the deuterated compound represented by Formula (A) according to claim 1 comprising:
(a) a step of forming a first reactant by dissolving the compound represented by Formula A in perdeuterated benzene (benzene-D₆);
(b) a step of forming a second reactant by adding deuterium-triflic acid (CF₃SO₃D) to the first reactant;
(c) a step of deuterating the second reactant by reacting the second reactant at 80° C. for 3 hours to 18 hours;
(d) a step of quenching by adding Na₂CO₃ in D₂O, after the reaction in step (c) is completed and the second reactant is cooled to room temperature, and
(e) a step of obtaining the deuterated compound represented by Formula A after concentration of the organic solvent of the second reactant and recrystallization with toluene and acetone solvent.

9. A method for preparing a deuterated compound represented by Formula (A) according to claim 1, wherein the compound represented by Formula (A) is synthesized by reacting a compound represented by Formula R1 or Formula R2 and a compound represented by Formula R3: 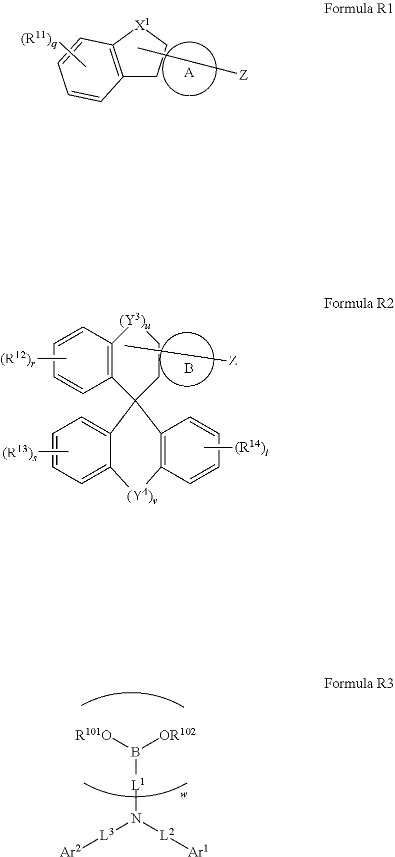
wherein:
1) L¹, L², L³, Ar¹ and Ar², are the same as defined in claim 1,
2) R¹¹, R¹², R¹³, and R¹⁴ are each independently selected from the group consisting of hydrogen; deuterium; halogen; a cyano group; a C₆-C₆₀ aryl group; a C₂-C₆₀ heterocyclic group including at least one heteroatom of O, N, S, Si or P; a fused ring group of a C₃-C₆₀ aliphatic ring and a C₆-C₆₀ aromatic ring: a C₁-C₅₀ alkyl group; a C₂-C₂₀ alkenyl group; a C₂-C₂₀ alkynyl group; a C₁-C₃₀ alkoxy group; a C₆-C₆₀ aryloxy group; and -L″-N(Ar^{a})(Ar^{b}); or R¹¹, R¹², R¹³, and R¹⁴ are bonded to adjacent groups to form a ring, wherein L″, Ar^{a} and Ar^{b} are the same as defined in claim 1,
ring A, ring B are each independently selected from the group consisting of a C₆-C₆₀ aryl group; a C₂-C₆₀ heterocyclic group including at least one heteroatom of O, N, S, Si or P; and a fused ring group of a C₃-C₆₀ aliphatic ring and a C₆-C₆₀ aromatic ring: or ring A and ring B may be substituted with R²¹, wherein R²¹ is defined the same as R¹¹ above,
q, r, s, and t are each independently an integer of 0 to 4, and
u and v are each independently 0 or 1 except that both u and v are 0, and where u or v is 0, Y³ or Y⁴ is absent,
3) R¹⁰¹ and R¹⁰² are each independently selected from the group consisting of a C₁-C₅₀ alkyl group; a C₂-C₂₀ alkenyl group; a C₂-C₂₀ alkynyl group; a C₁-C₃₀ alkoxyl group; and a C₆-C₃₀ aryloxy group; or R¹⁰¹ and R¹⁰² are bonded to adjacent groups to form a ring,
4) Z is halogen; or a triflate group;
5) w is 0 or 1.

10. An organic electric element comprising an anode, a cathode, and an organic material layer formed between the anode and the cathode, wherein the organic material layer comprises a single compound or 2 or more compounds represented by Formula A of claim 1.

11. The organic electric element of claim 10, wherein the organic material layer comprises at least one of a hole injection layer, a hole transport layer, an emitting auxiliary layer, an emitting layer, an electron transport auxiliary layer, an electron transport layer, and an electron injection layer.

12. The organic electric element of claim 10, wherein the organic material layer is an emitting auxiliary layer.

13. The organic electric element of claim 10, wherein the organic material layer is a hole transport layer.

14. The organic electric element of claim 10, wherein the organic electric element further comprises a light efficiency enhancing layer formed on at least one surface of the anode and the cathode, the surface being opposite to the organic material layer.

15. The organic electric element of claim 10, wherein the organic material layer comprises 2 or more stacks including a hole transport layer, an emitting layer, and an electron transport layer sequentially formed on the anode.

16. The organic electric element of claim 10, wherein the organic material layer further comprises a charge generation layer formed between the 2 or more stacks.

17. An electronic device comprising a display device comprising the organic electric element of claim 10; and a control unit for driving the display device.

18. An electronic device according to claim 17, wherein the organic electric element is at least one of an OLED, an organic solar cell, an organic photo conductor (OPC), organic transistor (organic TFT) and an element for monochromic or white illumination.
